(19) United States
(12) Patent Application Publication
(10) Pub. No.: US 2006/0089499 A1
(43) Pub. Date: Apr. 27, 2006

(54) Pyrazolopyrimidines and the use thereof for controlling harmful organisms

(76) Inventors: Olaf Gebauer, Koln (DE); Jorg Nico Greul, Leichlingen (DE); Herbert Gayer, Monheim (DE); Bernd-Wieland Kruger, Gladbach (DE); Hans-Ludwig Elbe, Wuppertal (DE); Ralf Dunkel, Monheim (DE); Oliver Guth, Leverkusen (DE); Arnd Voerste, Koln (DE); Stefan Hillebrand, Neuss (DE); Stefan Herrmann, Langenfeld (DE); Ulrich Heinemann, Leichlingen (DE); Ronald Ebbert, Monheim (DE); Karl-Heinz Kuck, Langenfeld (DE); Peter Losel, Leverkusen (DE); Ulrike Wachendorff-Neumann, Neuwied (DE); Astrid Mauler-Machnik, Leichlingen (DE)

Correspondence Address: NORRIS, MCLAUGHLIN & MARCUS, P.A., 875 THIRD AVE, 18TH FLOOR, NEW YORK, NY, 10022, US

(21) Appl. No.: 10/515,484

(22) PCT Filed: May 16, 2003
(86) PCT No.: PCT/EP03/05159

§ 371 Date: Nov. 4, 2005

(30) Foreign Application Priority Data
May 29, 2002 (DE) 102-23-917.7

(51) Int. Cl. C07D 487/04

(52) U.S. Cl. 544/281

(57) ABSTRACT

This invention relates to novel pyrazolopyrimidines of the formula 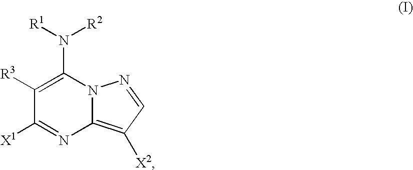 in which

R¹, R², R³, X¹ and X² are as defined in the disclosure, to a process for preparing these substances and to their use for controlling harmful organisms. This invention further relates to novel intermediates of the formulae 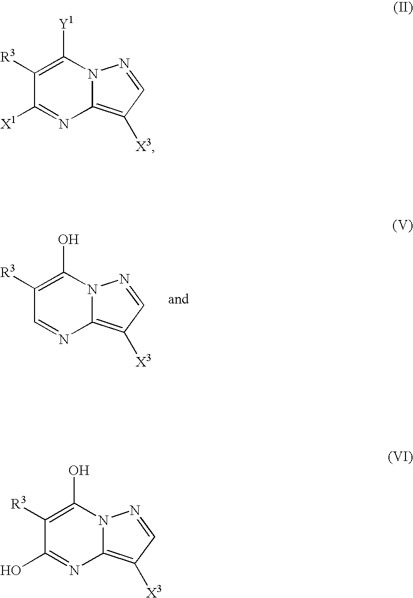 and to processes for their preparation.

The present invention relates to novel pyrazolopyrimidines, to a plurality of processes for their preparation and to their use for controlling harmful organisms. Moreover, the invention relates to novel intermediates and to processes for their preparation.

It is already known that certain pyrazolopyrimidines have fungicidal properties (compare DE-A 3 130 633 or FR-A 2 794 745). The activity of these substances is good; however, at low application rates it is sometimes unsatisfactory.

This invention now provides novel pyrazolopyrimidines of the formula 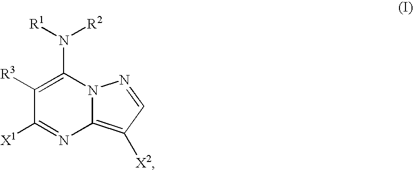
in which
R¹ represents amino, hydroxyl or represents in each case optionally substituted alkyl, alkenyl, alkynyl, cycloalkyl, alkoxy, alkenyloxy, alkinyloxy, cycloalkyloxy, alkylamino, dialkylamino, alkenylamino, alkinylamino, cyclo-alkylamino, N-cycloalkyl-N-alkylamino, alkylideneamino or heterocyclyl,
R² represents hydrogen or represents in each case optionally substituted alkyl, alkenyl, alkynyl or cycloalkyl, or
R¹ and R² together with the nitrogen atom to which they are attached form an optionally substituted heterocyclic ring,
R³ represents optionally substituted aryl,
X¹ represents hydrogen or halogen and
X² represents halogen, cyano, nitro, alkyl, haloalkyl, cycloalkyl, formyl, thiocarbamoyl, alkoxycarbonyl, alkylcarbonyl, hydroximinoalkyl or alkoximinoalkyl,
   and acid addition salts of those compounds of the formula (I),
   in which
R¹ represents amino.

Depending on the substitution pattern, the compounds according to the invention may, if appropriate, be present as mixtures of different possible isomeric forms, in particular of stereoisomers, such as, for example, E and Z, threo and erythro and optical isomers, and, if appropriate, also in the form of tautomers. If R³ carries different substituents on the two atoms adjacent to the point of attachment, the compounds in question may be present in a particular stereoisomeric form, that is, as atropi somers.

Furthermore, it has been found that pyrazolopyrimidines of the formula (I) can be prepared by

a) reacting halopyrazolopyrimidines of the formula 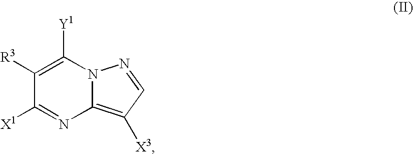
in which
R³ and X¹ are as defined above,
X³ represents halogen, cyano, nitro, alkyl, haloalkyl, cycloalkyl, thiocarbamoyl, alkoxycarbonyl or alkylcarbonyl and
Y¹ represents halogen
with amines of the formula 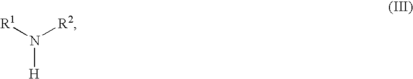
   in which
R¹ and R² are as defined above,
if appropriate in the presence of a diluent, if appropriate in the presence of a catalyst and if appropriate in the presence of an acid acceptor,
   or

b) reacting pyrazolopyrimidines of the formula (Ia) 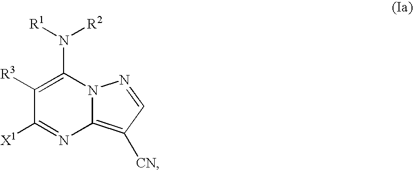
in which
R¹, R², R³ and X¹ are as defined above
with diisobutylaluminium hydride in the presence of aqueous ammonium chloride solution and in the presence of an organic diluent,
or

c) reacting pyrazolopyrimidines of the formula (Ib) 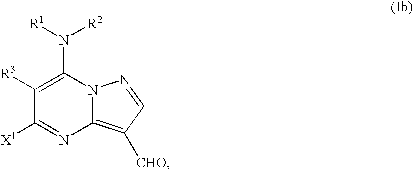
in which
R¹, R², R³ and X¹ are as defined above
with amino compounds of the formula (IV)
   H2N—OR⁴ (IV),
   in which
R⁴ represents hydrogen or alkyl,
in the presence of a diluent and if appropriate in the presence of a catalyst, where the amino compounds of the formula (IV) can also be employed in the form of their acid addition salts,
and, if appropriate, adding an acid to the resulting compounds of the formula (I), in which
R¹ represents amino.

Finally, it has been found that the novel pyrazolopyrimidines of the formula (I) and their acid addition salts are highly suitable for controlling harmful organisms. In particular, they have strong action against undesirable microorganisms, such as fungi and bacteria. Moreover, the substances according to the invention also have very good insecticidal and nematicidal action.

Surprisingly, the pyrazolopyrimidines of the formula (I) according to the invention and their acid addition salts have considerably better activity against harmful organisms than the constitutionally most similar substances of the prior art with the same direction of action.

The formula (I) provides a general definition of the pyrazolopyrimidines according to the invention.
R¹ preferably represents hydroxyl, amino, represents alkyl having 1 to 6 carbon atoms which is optionally substituted by halogen, cyano, hydroxyl, amino, phenyl, heterocyclyl, alkoxy having 1 to 4 carbon atoms, alkoxycarbonyl having 1 to 4 carbon atoms in the alkoxy moiety, alkylamino having 1 to 4 carbon atoms, dialkylamino having 2 to 8 carbon atoms, cycloalkyl having 3 to 6 carbon atoms, halocycloalkyl having 3 to 6 carbon atoms and 1 to 5 halogen atoms, alkylthio having 1 to 4 carbon atoms, oxo, hydroxyimino and/or alkoximino having 1 to 4 carbon atoms,
   represents optionally halogen-, cycloalkyl-, cyano-, phenyl- and/or heterocyclyl-substituted alkenyl having 2 to 6 carbon atoms,
   represents optionally halogen-, cycloalkyl-, cyano-, phenyl- and/or heterocyclyl-substituted alkynyl having 2 to 6 carbon atoms,
   represents optionally cycloalkyl having 3 to 7 carbon atoms which is optionally substituted by halogen, cycloalkyl, cyano, haloalkyl having 1 or 2 carbon atoms and 1 to 5 halogen atoms, phenyl and/or heterocyclyl,
   represents optionally halogen-, cycloalkyl-, cyano-, phenyl- and/or heterocyclyl-substituted alkoxy having 1 to 7 carbon atoms,
   represents optionally halogen-, cycloalkyl-, cyano-, phenyl- and/or heterocyclyl-substituted alkenyloxy having 2 to 6 carbon atoms,
   represents optionally halogen-, cycloalkyl-, cyano-, phenyl- and/or heterocyclyl-substituted alkynyloxy having 2 to 6 carbon atoms,
   represents optionally halogen-, cycloalkyl-, cyano-, phenyl- and/or heterocyclyl-substituted cycloalkyloxy having 3 to 7 carbon atoms,
   represents optionally halogen-, cycloalkyl-, cyano-, phenyl- and/or heterocyclyl-substituted alkylamino having 1 to 7 carbon atoms,
   represents optionally halogen-, cycloalkyl-, cyano-, phenyl- and/or heterocyclyl-substituted dialkylamino having 1 to 7 carbon atoms in each of the alkyl radicals,
   represents optionally halogen-, cycloalkyl-, cyano-, phenyl- and/or heterocyclyl-substituted alkenylamino having 2 to 6 carbon atoms,
   represents optionally halogen-, cycloalkyl-, cyano-, phenyl- and/or heterocyclyl-substituted alkynylamino having 2 to 6 carbon atoms,
   represents optionally halogen-, cycloalkyl-, cyano-, phenyl- and/or heterocyclyl-substituted cycloalkylamino having 3 to 7 carbon atoms,
represents optionally halogen-, cycloalkyl-, cyano-, phenyl- and/or heterocyclyl-substituted N-cycloalkyl-N-alkylamino having 3 to 7 carbon atoms in the cycloalkyl moiety and 1 to 7 carbon atoms in the alkyl moiety,
   represents optionally halogen-, cycloalkyl-, cyano-, phenyl- and/or heterocyclyl-substituted alkylideneamino having 2 to 6 carbon atoms, or
   represents optionally halogen-, alkyl-, cycloalkyl-, cyano-, phenyl- and/or heterocyclyl-substituted heterocyclyl having 5 or 6 ring members,
   where the heterocyclyl radicals mentioned above may be mono- to trisubstituted by identical or different substituents from the group consisting of
      halogen, hydroxy, phenyl, 1,2-dioxyethylene, alkyl having 1 to 4 carbon atoms, haloalkyl having 1 or 2 carbon atoms and 1 to 5 halogen atoms, alkoxy having 1 to 4 carbon atoms, alkylthio having 1 to 4 carbon atoms, haloalkoxy having 1 or 2 carbon atoms and 1 to 5 halogen atoms, haloalkylthio having 1 or 2 carbon atoms and 1 to 5 halogen atoms, where the heterocyclyl radicals mentioned above are saturated or partially unsaturated,
   and where the phenyl radicals mentioned above may be mono- to trisubstituted by identical or different substituents from the group consisting of
      halogen, cyano, nitro, amino, hydroxyl, formyl, carboxyl, carbamoyl, thiocarbamoyl;
      in each case straight-chain or branched alkyl, alkoxy, alkylthio, alkylsulphinyl or alkylsulphonyl having in each case 1 to 6 carbon atoms;
      in each case straight-chain or branched alkenyl or alkenyloxy having in each case 2 to 6 carbon atoms;
      in each case straight-chain or branched haloalkyl, haloalkoxy, haloalkylthio, haloalkylsulphinyl or haloalkylsulphonyl having in each case 1 to 6 carbon atoms and 1 to 13 identical or different halogen atoms;
      in each case straight-chain or branched haloalkenyl or haloalkenyloxy having in each case 2 to 6 carbon atoms and 1 to 13 identical or different halogen atoms;
      in each case straight-chain or branched alkylamino, dialkylamino, alkylcarbonyl, alkylcarbonyloxy, alkoxycarbonyl, alkylsulphonyloxy, hydroximinoalkyl or alkoximinoalkyl having in each case 1 to 6 carbon atoms in the individual alkyl moieties;
      cycloalkyl having 3 to 6 carbon atoms,
      1,3-propanediyl attached in the 2,3-position, 1,4-butanediyl, methylenedioxy (—O—CH2-O—) or 1,2-ethylenedioxy (—O—CH2-CH2-O—),
      where these radicals may be mono- or polysubstituted by identical or different substituents from the group consisting of halogen, alkyl having 1 to 4 carbon atoms and haloalkyl having 1 to 4 carbon atoms and 1 to 9 identical or different halogen atoms.
R² preferably represents hydrogen,
   represents alkyl having 1 to 4 carbon atoms which is optionally substituted by halogen, cycloalkyl having 3 to 6 carbon atoms, alkoxy having 1 to 4 carbon atoms, alkylthio having 1 to 4 carbon atoms, oxo, hydroximino and/or alkoximino having 1 to 4 carbon atoms,
   represents alkenyl having 2 to 4 carbon atoms which is optionally substituted by halogen and/or cycloalkyl having 3 to 6 carbon atoms,
   represents alkynyl having 2 to 4 carbon atoms which is optonally substituted by halogen and/or cycloalkyl having 3 to 6 carbon atoms or
   represents cycloalkyl having 3 to 6 carbon atoms which is optionally substituted by halogen and/or cycloalkyl having 3 to 6 carbon atoms.
R¹ and R² also preferably together with the nitrogen atom to which they are attached represent a 3- to 6-membered heterocyclic ring which is saturated or partially saturated, which, in addition to the nitrogen atom already mentioned, may contain a further heteroatom from the group consisting of nitrogen, oxygen and sulphur and which may be mono- to trisubstituted by identical or different substituents from the group consisting of
   halogen, hydroxyl, cyano, morpholinyl, amino, a fused phenyl ring, a methylene or ethylene bridge,
   alkyl having 1 to 4 carbon atoms,
   haloalkyl having 1 to 4 carbon atoms and 1 to 9 identical or different halogen atoms;
   alkylcarbonylamino having 1 to 4 carbon atoms in the alkyl moiety, dialkylamino having 2 to 8 carbon atoms,
   alkoxycarbonylamino having 1 to 4 carbon atoms in the alkoxy moiety,
   di(alkoxycarbonyl)amino having 2 to 8 carbon atoms in the alkoxy moieties,
   hydroxyalkyl having 1 to 4 carbon atoms,
   alkoxycarbonyl having 1 to 4 carbon atoms in the alkoxy moiety and
   alkylcarbonyl having 1 to 4 carbon atoms in the alkyl moiety.
R³ preferably represents phenyl which may be mono- to tetrasubstituted by identical or different substituents from the group consisting of
   halogen, cyano, nitro, amino, hydroxyl, formyl, carboxyl, carbamoyl, thiocarbamoyl;
   in each case straight-chain or branched alkyl, alkoxy, alkylthio, alkylsulphinyl or alkylsulphonyl having in each case 1 to 6 carbon atoms;
   in each case straight-chain or branched alkenyl or alkenyloxy having in each case 2 to 6 carbon atoms;
   in each case straight-chain or branched haloalkyl, haloalkoxy, haloalkylthio, haloalkylsulphinyl or haloalkylsulphonyl having in each case 1 to 6 carbon atoms and 1 to 13 identical or different halogen atoms;
   in each case straight-chain or branched haloalkenyl or haloalkenyloxy having in each case 2 to 6 carbon atoms and 1 to 11 identical or different halogen atoms;
   in each case straight-chain or branched alkylamino, dialkylamino, alkylcarbonyl, alkylcarbonyloxy, alkoxycarbonyl, alkylsulphonyloxy, hydroximinoalkyl or alkoximinoalkyl having in each case 1 to 6 carbon atoms in the individual alkyl moieties;
   cycloalkyl having 3 to 6 carbon atoms;
   1,3-propanediyl attached in the 2,3-position, 1,4-butanediyl methylenedioxy (—O—CH2-O—) or 1,2-ethylenedioxy (—O—CH2-CH2-O—), where these radicals may be mono- or polysubstituted by identical or different substituents from the group consisting of halogen, alkyl having 1 to 4 carbon atoms and/or haloalkyl having 1 to 4 carbon atoms and 1 to 9 identical or different halogen atoms.
X¹ preferably represents hydrogen, fluorine, chlorine or bromine.
X² preferably represents cyano, fluorine, chlorine, bromine, iodine, nitro, formyl, haloalkyl having 1 to 4 carbon atoms and 1 to 9 fluorine, chlorine and/or bromine atoms, alkyl having 1 to 4 carbon atoms, cycloalkyl having 3 to 6 carbon atoms, thiocarbamoyl, alkoxycarbonyl having 1 to 4 carbon atoms in the alkoxy moiety, alkylcarbonyl having 1 to 4 carbon atoms in the alkyl moiety, hydroximinoalkyl having 1 to 4 carbon atoms in the alkyl moiety or represents alkoxyiminoalkyl having 1 to 4 carbon atoms in the alkoxy moiety and 1 to 4 carbon atoms in the alkyl moiety.
R¹ particularly preferably represents hydroxyl, amino, methyl, ethyl, n-propyl, i-propyl, n-butyl, i-butyl, s-butyl, t-butyl, n-pentyl, i-pentyl, 1,2-dimethyl-propyl, 2,2-dimethylpropyl, 1,2,2-trimethylpropyl, or
R¹ particularly preferably represents methoxymethyl, 2-methoxyethyl, methylthiomethyl, 2-methylthioethyl, hydroximinomethyl, methoximino-methyl, acetylmethyl, 2-hydroximinopropyl, 2-methoximinopropyl, allyl, 2-methylprop-2-enyl, propargyl, 2,2,2-trifluoroethyl, 1-(trifluoromethyl)-ethyl, 3,3,3-trifluoropropyl, cyclopropylmethyl,
   cyclopropyloxy, cyclobutyloxy, cyclopentyloxy, cyclohexyloxy, difluoromethoxy, trifluoromethoxy, difluorochloromethoxy, trifluoroethoxy,
   methylamino, ethylamino, n- or i-propylamino, n-, i-, s- or t-butylamino, dimethylamino, diethylamino, trifluoroethylamino, cyclohexylmethylamino, 2-cyanoethylamino, allylamino, 1-cyclopropylethylamino, cyclopropylamino, cyclobutylamino, cyclopentylamino, cyclohexylamino, 1-methylethylideneamino,
   represents cyclopropyl, cyclobutyl, cyclopentyl, cyclohexyl, pyrrolidinyl, piperidinyl, morpholinyl, thiamorpholinyl, piperazinyl, each of which is
   optionally mono- or disubstituted by identical or different substituents from the group consisting of fluorine, chlorine and methyl, or
   represents optionally substituted pyridylmethyloxy or thiazolylmethoxy,
   or
R¹ particularly preferably represents (2,2-dichlorocyclopropyl)methyl, (2-furyl)-methyl, (2-tetrahydrofuryl)methyl, (2-tetrahydropyranyl)methyl, 1,3-dioxolan-2-ylmethyl, 1-cyclopropylethyl, benzyloxy, 2,4-dichlorobenzyloxy, 2,6-dichlorobenzyloxy, 2-chlorobenzyloxy, 2-fluorocyclopropyl, 2-hexa-hydropyranyloxy, 2-thienylmethyl, 2-trifluoromethylcyclohexyl, 3-(dimethylamino)propyl, 3,5-bistrifluoromethylcyclohexyl, 3,5-dichloro-benzyloxy, 3-aminopropyl, 3-chlorobenzyloxy, 3-trifluoromethylbenzyloxy, 3-trifluoromethylcyclohexyl, 4-trifluoromethylcyclohexyl, 4-chlorobenzyloxy, 4-fluorobenzyloxy, 4-trifluoromethylbenzyloxy, —C(CH3)2-CF3, —C(CH3)2-CH2-COCH3, —CH(CH2OH)—COOCH3, —CH(CH3)-CH(O—CH3)2, —CH(CH3)-CH═CH2, —CH(CH3)-CH2-CH(CH3)2, —CH(CH3)-CH2-O—CH3, —CH(CH3)-CH2-OH, —CH(CH3)-COOCH3, —CH(CH3)-COO-t-butyl, —CH2-C(CH3)=CH2, —CH2-CH(OCH3)2, —CH2-CH2-CF3, —CH2-CH2-Cl, —CH2-CH2-CN, —CH2-CH2-N(CH3)2, —CH2-CH2-N(CH3)2, —CH2-CH2-NH2, —CH2-CHF2, —CH2-CN, —CH2-COOC2H5, —CH2-COOCH3, i-butoxy, —NH—CH2-CF2-CHF2, —NH—CH2-CF3, —NH—CH2-CH(CH3)2, methoxy, ethoxy, i-propoxy, t-butoxy or —O—CH(CH3)-CH2-CH3,
   where the abovementioned thiazolyl and pyridyl radicals may be substituted, in the case of thiazolyl mono- or disubstituted and in the case of pyridyl mono- to trisubstituted, in each case by identical or different substituents from the group consisting of fluorine, chlorine, bromine, methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, difluoromethoxy, trifluoromethoxy, difluorochloromethoxy, trifluoroethoxy, difluoromethylthio, difluorochloromethylthio, dichlorfluoromethylthio, trifluoromethylthio and phenyl,
   and where the benzyloxy radicals mentioned above may be mono- to trisubstituted in the phenyl moiety by identical or different substituents from the group consisting of
   fluorine, chlorine, bromine, cyano, nitro, amino, hydroxyl, formyl, carboxyl, carbamoyl, thiocarbamoyl, methyl, ethyl, n- or i-propyl, n-, i-, s- or t-butyl, methoxy, ethoxy, n- or i-propoxy, methylthio, ethylthio, n- or i-propylthio, methylsulphinyl, ethylsulphinyl, methylsulphonyl or ethylsulphonyl, trifluoromethyl, trifluoroethyl, difluoromethoxy, trifluoromethoxy, difluorochlormethoxy, trifluoroethoxy, difluoromethylthio, difluorochloro-methylthio, trifluoromethylthio, trifluoromethylsulphinyl, trifluoromethyl-sulphonyl, methylamino, ethylamino, n- or i-propylamino, dimethylamino, diethylamino, acetyl, propionyl, acetyloxy, methoxycarbonyl, ethoxycarbonyl, methylsulphonyloxy, ethylsulphonyloxy, hydroximinomethyl, hydroximino-ethyl, methoximinomethyl, ethoximinomethyl, methoximinoethyl, ethoximinoethyl, cyclopropyl, cyclobutyl, cyclopentyl or cyclohexyl,
   1,3-propanediyl attached in the 2,3-position, methylenedioxy (—O—CH₂—O—) or 1,2-ethylenedioxy (—O—CH₂—CH₂—O—), where these radicals may be mono- or polysubstituted by identical or different substituents from the group consisting of fluorine, chlorine, methyl, ethyl, n-propyl, i-propyl and trifluoromethyl.
R² particularly preferably represents hydrogen, methyl, ethyl, n- or i-propyl, n-, i-, s- or t-butyl, methoxymethyl, 2-methoxyethyl, methylthiomethyl, 2-methylthioethyl, hydroximinomethyl, methoximinomethyl, acetylmethyl, 2-hydroxyiminopropyl, 2-methoxyiminopropyl, allyl, propargyl, 2,2,2-trifluoroethyl, 1-(1,1,1-trifluoromethyl)ethyl, cyclopropylmethyl, cyclobutylmethyl, cyclopentylmethyl or cyclohexylmethyl.
R¹ and R² particularly preferably together with the nitrogen atom to which they are attached represent 1-pyrrolinyl, 3-pyrrolinyl, pyrrolidinyl, dihydropyridinyl, piperidinyl, pyrazolinyl, pyrazolidinyl, imidazolinyl, imidiazolidinyl, 1,2-diazinanyl, 1,3-diazinanyl, piperazinyl, oxazolinyl, oxazolidinyl, isoxazolyl, isoxazolidinyl, tetrahydropyridazinyl, dihydrooxazinyl, morpholinyl, thiazolinyl, thiazolidinyl or thiomorpholinyl, where the heterocycles mentioned may be substituted by
   fluorine, chlorine, bromine, cyano, nitro, amino, hydroxyl, formyl, carboxyl, carbamoyl, thiocarbamoyl, methyl, ethyl, n- or i-propyl, n-, i-, s- or t-butyl, methoxy, ethoxy, n- or i-propoxy, methylthio, ethylthio, n- or i-propylthio, methylsulphinyl, ethylsulphinyl, methylsulphonyl or ethylsulphonyl, trifluoromethyl, trifluoroethyl, difluoromethoxy, trifluoromethoxy, difluoro-chloromethoxy, trifluoroethoxy, difluoromethylthio, difluorochloromethyl-thio, trifluoromethylthio, trifluoromethyl sulphinyl, trifluoromethyl sulphonyl, methylamino, ethylamino, n- or i-propylamino, dimethylamino, diethylamino, acetyl, propionyl, acetyloxa, methoxycarbonyl, ethoxycarbonyl, methyl-sulphonyloxy, ethylsulphonyloxy, hydroximinomethyl, hydroximinoethyl, methoximinomethyl, ethoximinomethyl, methoximinoethyl, ethoximinoethyl, cyclopropyl, cyclobutyl, cyclopentyl, cyclohexyl,
   by a fused phenyl ring or
   by a methanediyl or ethanediyl bridge,
   or
R¹ and R² particularly preferably together represent a grouping of the formula

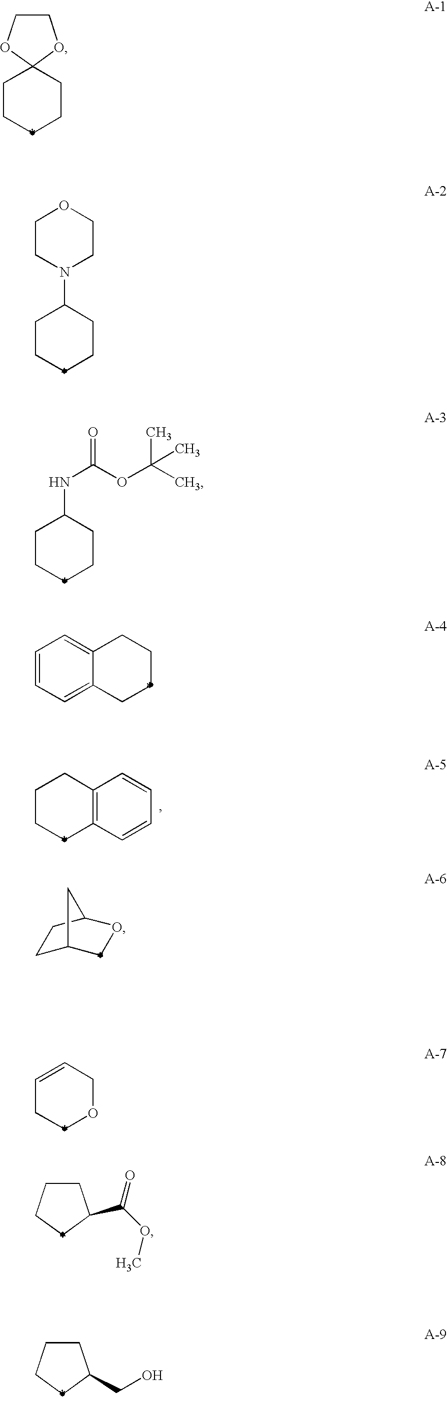

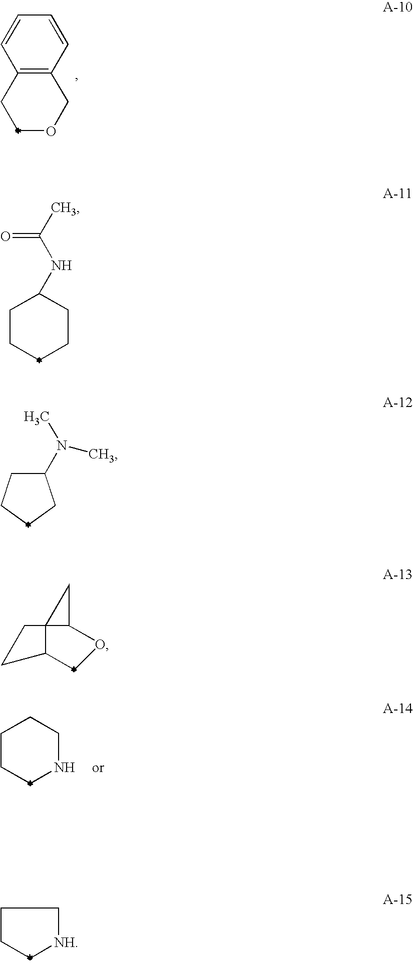

In these groups, the position attached to the nitrogen atom is in each case marked by *.
R³ particularly preferably represents phenyl which is mono- to trisubstituted by identical or different substituents from the group consisting of
   fluorine, chlorine, bromine, cyano, nitro, formyl, methyl, ethyl, n- or i-propyl, n-, i-, s- or t-butyl, allyl, propargyl, methoxy, ethoxy, n- or i-propoxy, methylthio, ethylthio, n- or i-propylthio, methylsulphinyl, ethylsulphinyl, methylsulphonyl, ethylsulphonyl, allyloxy, propargyloxy, trifluoromethyl, trifluoroethyl, difluoromethoxy, trifluoromethoxy, difluorchlormethoxy, trifluorethoxy, difluoromethylthio, difluorochloromethylthio, trifluoro-methylthio, trifluoromethylsulphinyl, trifluoromethylsulphonyl, trichloro-ethynyloxy, trifluoroethynyloxy, chloroallyloxy, iodopropargyloxy, methylamino, ethylamino, n- or i-propylamino, dimethylamino, diethylamino, acetyl, propionyl, acetyloxy, methoxycarbonyl, ethoxycarbonyl, hydroximinomethyl, hydroximinoethyl, methoximinomethyl, ethoximino-methyl, methoximinoethyl, ethoximinoethyl, cyclopropyl, cyclobutyl, cyclopentyl or cyclohexyl,
   1,3-propanediyl attached in the 2,3-position, methylenedioxy (—O—CH₂—O—) or 1,2-ethylenedioxy (—O—CH₂—CH₂—O—), where these radicals may be mono- or polysubstituted by identical or different radicals from the group consisting of fluorine, chlorine, methyl, ethyl, n-propyl, i-propyl and/or trifluoromethyl.
X¹ particularly preferably represents hydrogen, fluorine or chlorine.
X² particularly preferably represents cyano, fluorine, chlorine, bromine, iodine, formyl, trifluoromethyl, methoxycarbonyl, methylcarbonyl, hydroximinomethyl, methoximinomethyl, thiocarbamoyl, nitro, methyl, ethyl or cyclopropyl. R³ very particularly preferably represents 2,4-, 2,5- or 2,6-disubstituted phenyl, or represents 2-substituted phenyl or represents 2,4,6-trisubstituted phenyl.

A very particularly preferred group are the compounds of the formula (I), in which
R¹ represents amino, hydroxyl, methyl, ethyl, n-propyl, i-propyl, n-butyl, i-butyl, s-butyl, t-butyl, 1,2-dimethylpropyl, 1,2,2-trimethylpropyl, 2,2-dimethyl-propyl, trifluoromethyl, 2,2,2-trifluoromethyl, 2,2-difluoroethyl, 2,2,2-trifluoro-1-methylethyl, 3,3,3-trifluoropropyl, 2,2,2-trifluoro-1,1-dimethyl-ethyl, 3-methyl-butyl, allyl, 2-methyl-prop-2-enyl, 2-methoxyethyl, 2,2-dimethoxyethyl, cyclopropyl, cyclopentyl, cyclohexyl, 2-fluorocyclopropyl, 2-trifluoromethylcyclohexyl, 3-trifluoromethylcyclohexyl, 4-trifluoromethyl-cyclohexyl, 3,5-di(trifluoromethyl)cyclohexyl, cyclopropylmethyl, dichloro-cyclopropylmethyl, 1-cyclohexylethyl, 2-furylmethyl, 2-tetrahydrofuryl-methyl, 2-thienylmethyl, 1,3-dioxolan-2-ylmethyl, propargyl, methoxy-carbonylmethyl, ethoxycarbonylmethyl, 2-aminoethyl, 3-aminopropyl, 2-dimethylaminoethyl, cyanomethyl, 2-cyanoethyl, 2-vinyloxyethyl, pyrrolidinyl, piperidinyl, morpholinyl, thiomorpholinyl or piperazinyl,
R² represents hydrogen, methyl, ethyl, n- or i-propyl, n-, i-, s- or t-butyl, allyl, propargyl, 2,2,2-trifluoroethyl, 1-(1,1,1-trifluoromethyl)ethyl, cyclopropylmethyl, cyclobutylmethyl, cyclopentylmethyl, cyclohexylmethyl or cyclopropyl or
R¹ and R² together with the nitrogen atom to which they are attached represent pyrrolidinyl, piperidinyl, morpholinyl, thiomorpholinyl, piperazinyl, 5-methyl-3,6-dihydro-1(2H)-pyridinyl, 5-ethyl-3,6-dihydro-1-(2H)-pyridinyl or tetrahydro-1-(2H)-pyridazinyl, each of which is optionally mono- or disubstituted by identical or different substituents from the group consisting of fluorine, chlorine, bromine, cyano, hydroxyl, methyl, ethyl, trifluoromethyl, methylcarbonyl, methylcarbonylamino or methoxycarbonyl, or represent a grouping of the formula 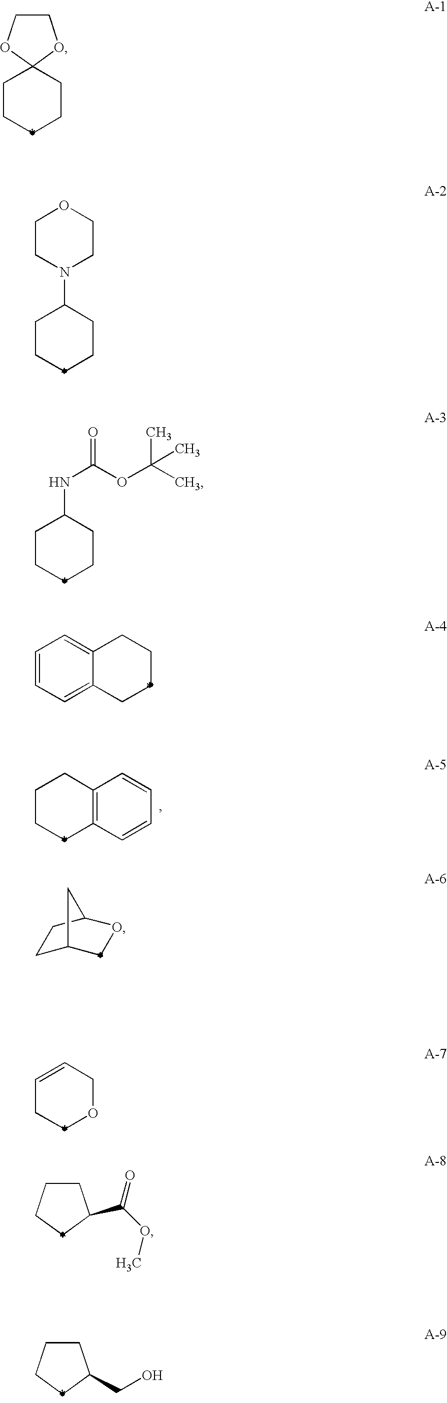 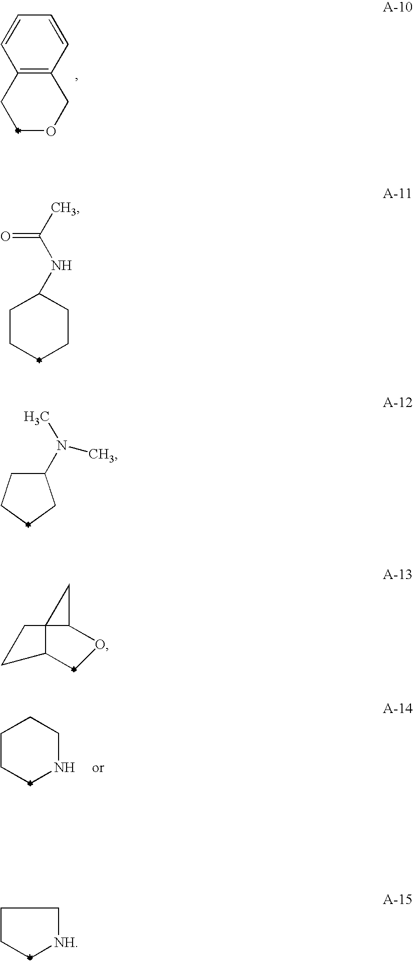
R³ represents phenyl which is mono- to trisubstituted in positions 2, 4 and/or 6 by fluorine and/or chlorine,
or
R³ represents 2-trifluoromethylphenyl, 2-chloro-5-nitrophenyl or 2-chloro-4-methoxyphenyl,
X¹ represents hydrogen or chlorine and
X² represents fluorine, chlorine, bromine, iodine, cyano, nitro, methyl, cyclopropyl, formyl, thiocarbamoyl or methoximinomethyl.

The radical definitions mentioned above can be combined with one another as desired. Moreover, individual definitions may not apply.

Compounds which are preferred according to the invention include addition products of acids and those pyrazolopyrimidines of the formula (I), in which
R¹ represents amino and
R², R³, X¹ and X² have the meanings mentioned as being preferred for these radicals.

The acids which may be added preferably include hydrohalic acids, such as, for example, hydrochloric acid and hydrobromic acid, in particular hydrochloric acid, furthermore phosphoric acid, nitric acid, mono- and bifunctional carboxylic acids and hydroxycarboxylic acids, such as, for example, acetic acid, maleic acid, succinic acid, fumaric acid, tartaric acid, citric acid, salicylic acid, sorbic acid and lactic acid, and also sulfonic acids, such as, for example, p-toluenesulphonic acid, 1,5-naphthalenedisulphonic acid, saccharin and thiosaccharin.

The general or preferred radical definitions listed above apply both to the end products of the formula (I) and, correspondingly, to the starting materials and intermediates required in each case for the preparation.

Using 3-cyano-5,7-dichloro-6-(2-chlorophenyl)pyrazolo[1,5-a]pyrimidine and methylethylamine as starting materials, the course of the process (a) according to the invention can be illustrated by the formula scheme below.

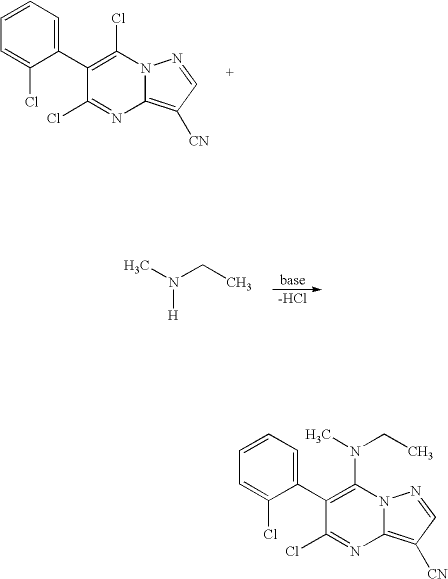

Using 3-cyano-5-chloro-6-(2-chloro-4-fluorophenyl)-7-(1,2,2-trimethylpropyl-amino)pyrazolo[1,5a]pyrimidine as starting material and diisobutylaluminium hydride as reaction component, the course of the process (b) according to the invention can be illustrated by the formula scheme below.

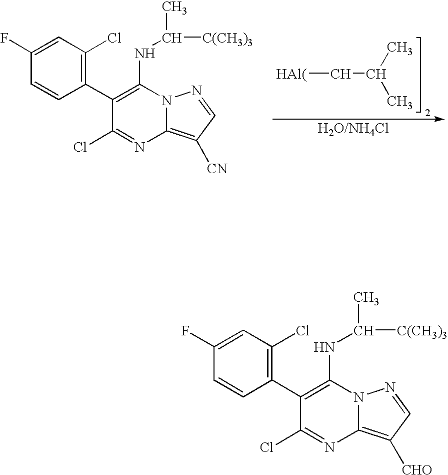

Using 3-formyl-5-chloro-6-(2-chloro-4-fluorophenyl)-7-(1,2,2-trimethylpropyl-amino)pyrazolo[1,5a]pyrimidine and methoxyamine hydrochloride as starting materials, the course of the process (c) according to the invention can be illustrated by the formula scheme below.

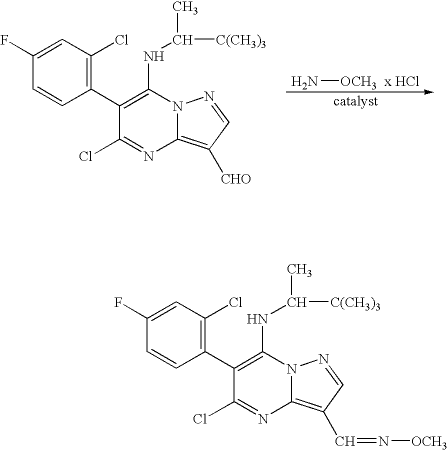

The formula (II) provides a general definition of the halopyrazolopyrimidines required as starting materials for carrying out the process (a) according to the invention. In this formula (II), R³ and X¹ preferably have those meanings which have already been mentioned in connection with the description of the compounds of the formula (I) according to the invention as being preferred for these radicals. Y¹ preferably represents fluorine, chlorine or bromine, particularly preferably fluorine or chlorine.
X³ preferably represents cyano, fluorine, chlorine, bromine, iodine, nitro, haloalkyl having 1 to 4 carbon atoms and 1 to 9 fluorine, chlorine and/or bromine atoms, alkyl having 1 to 4 carbon atoms, cycloalkyl having 3 to 6 carbon atoms, thiocarbamoyl, alkoxycarbonyl having 1 to 4 carbon atoms in the alkoxy moiety or alkylcarbonyl having 1 to 4 carbon atoms in the alkyl moiety.
X³ particularly preferably represents cyano, fluorine, chlorine, bromine, iodine, trifluoromethyl, methoxycarbonyl, methylcarbonyl, thiocarbamoyl, nitro, methyl, ethyl or cyclopropyl.
X³ very particularly preferably represents fluorine, chlorine, bromine, iodine, cyano, nitro, methyl, cyclopropyl or thiocarbamoyl.

The halopyrazolopyrimidines of the formula (II) are novel. These substances, too, are suitable for controlling pests, in particular for controlling unwanted microorganisms.

The halopyrazolopyrimidines of the formula (II) can be prepared

by

d) reacting hydroxypyrazolopyrimidines of the formula 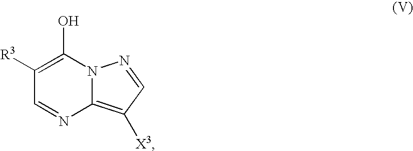
in which
R³ and X³ are as defined above
with halogenating agents, if appropriate in the presence of a diluent,
   or

e) reacting dihydroxypyrazolopyrimidines of the formula 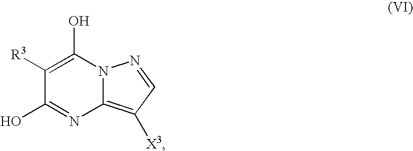
in which
R³ and X³ are as defined above
with halogenating agents, if appropriate in the presence of a diluent.

The formula (V) provides a general definition of the hydroxypyrazolopyrimidines required as starting materials for carrying out the process (d) according to the invention. In this formula, R³ and X³ preferably have those meanings which have already been mentioned in connection with the description of the compounds of the formulae (I) and (II) as being preferred for these radicals.

The hydroxypyrazolopyrimidines of the formula (V), too, have hitherto not been disclosed. They can be prepared by

f) reacting acrylic acid esters of the formula 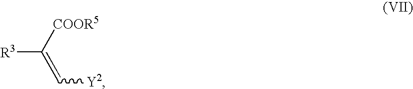
in which
R³ is as defined above,
R⁵ represents alkyl and
Y² represents alkoxy or dialkylamino,
with aminopyrazoles of the formula 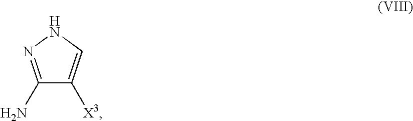
   in which
X³ is as defined above,
if appropriate in the presence of a diluent and if appropriate in the presence of a strong base.

The formula (VII) provides a general definition of the acrylic acid esters required as starting materials for carrying out the process (f) according to the invention. In this formula, R³ preferably has those meanings which have already been mentioned in connection with the description of the substances of the formula (I) according to the invention as being preferred for this radical. R⁵ preferably represents alkyl having 1 to 4 carbon atoms, particularly preferably methyl or ethyl. Y² preferably represents alkoxy having 1 to 4 carbon atoms or represents dialkylamino having 1 to 4 carbon atoms in each alkyl group. Particularly preferably, Y² represents methoxy, ethoxy or represents dimethylamino.

The acrylic acid esters of the formula (VII) are known or can be prepared by known methods (cf. EP-A 0 165 448).

The formula (VIII) provides a general definition of the aminopyrazoles required as reaction components for carrying out the process (f) according to the invention. In this formula, X³ preferably has those meanings which have already been mentioned in connection with the description of the substances of the formula (II) according to the invention as being preferred for this substituent.

The aminopyrazoles of the formula (VIII) are known or can be prepared by known methods (cf. Tetrahedron Lett. 21, 2029-2031 (1967); Liebigs Ann. Chem. 707, 141-146 (1967), Monatsh. Chem. 1998, 1329 (12), 1313-1318) and J. Med. Chem. 25 (1982), 239 ff).

The formula (VI) provides a general definition of the dihydroxypyrazolopyrimidines required as starting materials for carrying out the process (e) according to the invention. In this formula, R³ and X³ preferably have those meanings which have already been mentioned in connection with the description of the substances of the formulae (I) and (II) according to the invention as being preferred for these radicals.

The dihydroxypyrazolopyrimidines of the formula (VI), too, have hitherto not been disclosed. They can be prepared by

g) reacting malonic esters of the formula 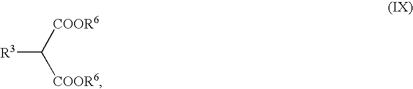
in which
R³ is as defined above and
R⁶ represents alkyl,
with aminopyrazoles of the formula 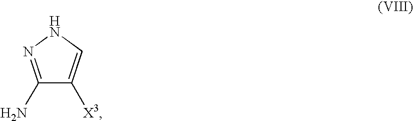
   in which
X³ is as defined above,
if appropriate in the presence of a diluent and if appropriate in the presence of a strong base.

The formula (IX) provides a general definition of the malonic esters required as starting materials for carrying out the process (g) according to the invention. In this formula, R³ preferably has those meanings which have already been mentioned in connection with the description of the substances of the formula (I) according to the invention as being preferred for this radical. R⁶ preferably represents alkyl having 1 to 4 carbon atoms, particularly preferably methyl or ethyl.

The malonic esters of the formula (IX) are known or can be prepared by known methods (cf. U.S. Pat. No. 6,156,925).

Suitable diluents for carrying out the processes (f) and (g) are all customary inert organic solvents. Preference is given to using aliphatic, alicyclic or aromatic hydrocarbons, such as petroleum ether, hexane, heptane, cyclohexane, methylcyclo-hexane, benzene, toluene, xylene or decalin; halogenated hydrocarbons, such as chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethyl-formamide, N,N-dimethylacetamide, N-methylformanilide, N-methylpyrrolidone or hexamethylphosphoric triamide; esters, such as methyl acetate or ethyl acetate; sulphoxides, such as dimethyl sulphoxide; sulphones, such as sulpholane; alcohols, such as methanol, ethanol, n- or i-propanol, n-, i-, sec- or tert-butanol, ethanediol, propane-1,2-diol, ethoxyethanol, methoxyethanol, diethylene glycol monomethyl ether, diethylene glycol monoethyl ether; amines, such as tri-n-butylamine; or carboxylic acids, such as acetic acid.

Suitable strong bases for carrying out the processes (f) and (g) according to the invention are, preferably, alkaline earth metal or alkali metal hydrides or alkoxides and also alkali metal amides. Sodium hydride, sodium amide, sodium methoxide, sodium ethoxide and potassium tert-butoxide may be mentioned by way of example.

The processes (f) and (g) according to the invention and also the other processes according to the invention are generally carried out under atmospheric pressure. However, it is also possible to operate under elevated pressure or—unless highly volatile reaction components are present—under reduced pressure.

When carrying out the processes (f) and (g) according to the invention, the reaction temperatures can in each case be varied within a relatively wide range. In the absence of bases, the processes are generally carried out at temperatures between 100° C. and 250° C., preferably between 120° C. and 200° C. If bases are present, the processes are generally carried out at temperatures between 20° C. and 120° C., preferably between 20° C. and 80° C.

When carrying out the process (f) according to the invention, in general 1 to 15 mol, preferably 1 to 8 mol, of aminopyrazole of the formula (VIII) are employed per mole of acrylic acid ester of the formula (VII). Work-up is carried out by customary methods.

When carrying out the process (g) according to the invention, in general 1 to 15 mol, preferably 1 to 8 mol, of aminopyrazole of the formula (VIII) are employed per mole of malonic ester of the formula (IX). Work-up is carried out by customary methods.

Suitable halogenating agents for carrying out the processes (d) and (e) according to the invention are in each case all customary reagents suitable for exchanging hydroxyl groups attached to carbon for halogen. Preference is given to using phosphorus trichloride, phosphorus tribromide, phosphorus pentachloride, phosphorus oxychloride, phosgene, thionyl chloride, thionyl bromide or mixtures thereof. The corresponding fluoro compounds of the formula (II) can be prepared from the chloro or bromo compounds by reaction with potassium fluoride.

Suitable diluents for carrying out the processes (d) and (e) according to the invention are in each case all organic solvents customary for such halogenations. Preference is given to using aliphatic, alicyclic or aromatic hydrocarbons, such as petroleum ether, hexane, heptane, cyclohexane, methylcyclohexane, benzene, toluene, xylene or decalin; halogenated hydrocarbons, such as chlorobenzene, dichlorobenzene, dichloromethane, chloroform, carbon tetrachloride, dichloroethane or trichlorethane.

However, the diluent used can also be the halogenating agent itself or a mixture of halogenating agent and one of the diluents mentioned.

When carrying out the processes (d) and (e) according to the invention, the reaction temperatures can in each case be varied within a relatively wide range. In general, the processes are carried out at temperatures between 20° C. and 150° C., preferably between 40° C. and 120° C.

When carrying out the processes (d) and (e) according to the invention, in each case an excess of halogenating agent is used per mole of hydroxypyrazolopyrimidine of the formula (V) and dihydroxypyrazolopyrimidine of the formula (VI), respectively. Work-up is in each case carried out by customary methods.

The formula (III) provides a general definition of the amines further required as starting materials for carrying out the process (a) according to the invention. In this formula, R¹ and R² preferably have those meanings which have already been mentioned in connection with the description of the compounds of the formula (I) according to the invention as being preferred for R¹ and R².

Some of the amines of the formula (III) are known.

Amines of the formula 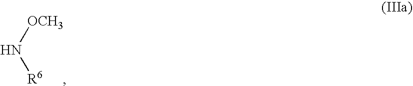
in which
R¹ represents isobutyl, 2-methoxyethyl or represents 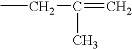
   are novel.

The amines of the formula (IIIa) can be prepared by

h) reacting, in a first step, ethyl N-methoxycarbamate of the formula 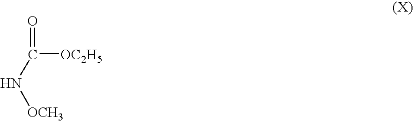
with halogen compounds of the formula
R⁷—X⁴ (XI),
in which
R⁷ is as defined above and
X⁴ represents bromine or iodine
in the presence of a base or in the presence of a diluent and reacting the resulting carbamates of the formula 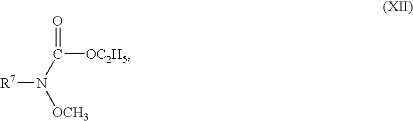
   in which
R⁷ is as defined above,
in a second step with potassium hydroxide in the presence of ethanol and water.

Amines of the formula 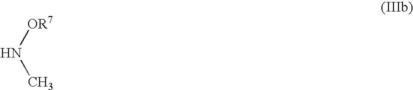
in which
R⁷ is as defined above
are also novel.

The amines of the formula (IIIb) can be prepared by

i) reacting, in a first step, ethyl N-hydroxy-N-methylcarbamate of the formula 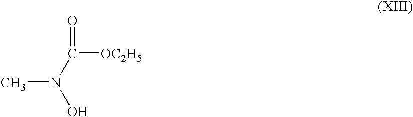
with halogen compounds of the formula
R⁷—X⁴ (XI),
in which
R⁷ and X⁴ are as defined above
in the presence of a base and in the presence of a diluent and reacting the resulting carbamates of the formula 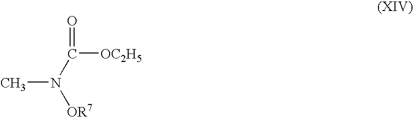
   in which
R⁷ is as defined above
in a second step with potassium hydroxide in the presence of ethanol and water.

Trifluoroisopropylamines of the formula 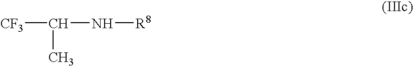
in which
R⁸ represents methyl, ethyl or propyl
   are also novel.

The trifluoroisopropylamines of the formula (IIIc) can be prepared by

j) reacting, in a first step, ethyl N-trifluoroisopropylcarbamate of the formula 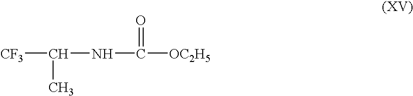
with halogen compounds of the formula
R⁸—X⁴ (XVI)
in which
R⁸ and X⁴ are as defined above
in the presence of a base and in the presence of a diluent and reacting the resulting carbamates of the formula 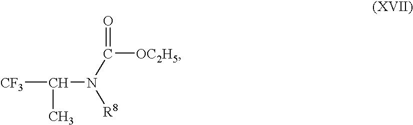
   in which
R⁸ is as defined above,
in a second step with potassium hydroxide in the presence of ethanol and water.

Finally, the 3-trifluoromethyl-3-aminopropene of the formula 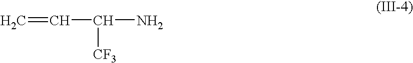
is also novel.

The 3-trifluoromethyl-3-aminopropene of the formula (III-4) can be prepared by

k) reacting the carbamate of the formula 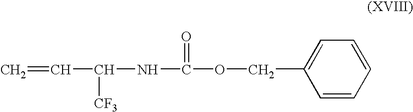
with aqueous hydrochloric acid.

The compounds of the formulae (X), (XI), (XIII), (XV), (XVI) and (XVIII) required as starting materials for carrying out the processes (h)-(j) are known or can be prepared by known methods.

Suitable acid acceptors for carrying out the first step of the processes (h), (i) and (j) according to the invention are in each case all inorganic and organic acid acceptors customary for such reactions.

Preference is given to using alkaline earth metal or alkali metal hydrides, hydroxides, amides, alkoxides, acetates, carbonates or bicarbonates, such as, for example, sodium hydride, sodium amide, sodium methoxide, sodium ethoxide, potassium tert-butoxide, sodium hydroxide, potassium hydroxide, sodium acetate, potassium acetate, calcium acetate, sodium carbonate, potassium carbonate, potassium bicarbonate and sodium bicarbonate, and furthermore ammonium compounds, such as ammonium hydroxide, ammonium acetate and ammonium carbonate. Suitable organic bases which may be mentioned are: tertiary amines, such as trimethylamine, triethyl amine, tributyl amine, N,N-dimethyl aniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

Suitable diluents for carrying out the first step of the processes (h), (i) and (j) are in each case all customary inert organic solvents. Preference is given to using ethers, such as diethyl ether, dioxane, tetrahydrofuran, 1,2-dimethoxyethane, 1,2-diethoxyethane or anisole; amides, such as N,N-dimethylformamide, N,N-dimethylacetamide, N-methylformanilide or N-methylpyrrolidone; sulphones, such as sulpholane; alcohols, such as methanol, ethanol, isopropanol, tert-butanol, n-butanol.

When carrying out the first step of the processes (h), (i) and (j), the reaction temperatures can in each case be varied within a relatively wide range. In general, the first step is carried out at temperatures between 0° C. and 150° C., preferably between 10° C. and 100° C.

The first step of the processes (h), (i) and (j) is in each case generally carried out under atmospheric pressure. However, it is also possible to operate under elevated pressure or, if no low-boiling components are involved in the reaction, under reduced pressure.

For carrying out the first step of the processes (h), (i) and (j),
in general from 0.5 to 15 mol, preferably from 1 to 5 mol, of halogen compound of the formula (XI) are employed per mole of ethyl N-methoxycarbamate of the formula (X), or
in general from 0.5 to 15 mol, preferably from 1 to 5 mol, of halogen compound of the formula (XI) are employed per mole of ethyl N-hydroxy-N-methylcarbamate of the formula (XIII), or
in general from 0.5 to 15 mol, preferably from 1 to 5 mol, of halogen compound of the formula (XVI) are employed per mole of ethyl N-trifluoroisopropyl-carbamate of the formula (XV).

Work-up is in each case carried out by customary methods, for example by extraction and subsequent drying or by precipitation with subsequent filtration and drying. Any impurities that may still be present can be removed by customary methods.

The compounds of the formulae (XI), (XIV) and (XVII) obtained as intermediates when carrying out the first step of the processes (h), (i) and (j) are novel.

When carrying out the second step of processeses (h), (i) and (j), the reaction temperatures can also in each case be varied within a relatively wide range. In general, the second step is carried out at temperatures between 0° C. and 100° C., preferably between 10° C. and 80° C.

The second step of the processes (h), (i) and (j), too, is generally in each case carried out under atmospheric pressure. However, again it is in each case also possible to operate under elevated pressure or, unless the products to be isolated have very low boiling points, under reduced pressure.

When carrying out the second step of the processes (h), (i) and (j), in each case up to 10 mol of potassium hydroxide are employed per mole of a compound of the formula (XII), (XIV) or (XVII). Work-up is carried out by customary methods. Here, the amines are generally expediently isolated in the form of their salts by adding acid, preferably aqueous hydrochloric acid.

When carrying out the process (k), the reaction temperatures can likewise be varied within a relatively wide range. In general, the process is carried out a temperatures between 10° C. and 150° C., preferably at reflux temperature.

The process (k) is generally carried out under atmospheric pressure. However, it is also possible to operate under elevated pressure.

When carrying out the process (k), an excess, preferably up to 10 mol, of aqueous hydrochloric acid is employed per mole of carbamate of the formula (XVM). Work-up is again carried out by customary methods.

Suitable diluents for carrying out the process (a) according to the invention are all customary inert organic solvents. Preference is given to using aliphatic, alicyclic or aromatic hydrocarbons, such as petroleum ether, hexane, heptane, cyclohexane, methylcyclohexane, benzene, toluene, xylene or decalin; halogenated hydrocarbons, such as chlorobenzene, dichlorobenzene, dichloromethane, chloroform, carbon tetrachloride, dichloroethane or trichloroethane; ethers, such as diethyl ether, diisopropyl ether, methyl t-butyl ether, dioxane, tetrahydrofuran, 1,2-dimethoxy-ethane or 1,2-diethoxyethane; amides, such as N,N-dimethylformamide, N,N-dimethylacetamide or N-methylpyrrolidone; esters, such as methyl acetate or ethyl acetate; sulphoxides, such as dimethyl sulphoxide; sulphones, such as sulpholane.

Suitable catalysts for carrying out the process according to the invention are all reaction accelerators customary for such reactions. Preference is given to using alkali metal fluorides, such as potassium fluoride or caesium fluoride.

Suitable acid acceptors for carrying out the process (a) according to the invention are all acid binders customary for such reactions. Preference is given to using ammonia and also tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

When carrying out the process (a) according to the invention, the reaction temperatures can be varied within a relatively wide range. In general, the process is carried out at temperatures between 0° C. and 150° C., preferably at temperatures between 0° C. and 80° C.

When carrying out the process (a) according to the invention, in general from 0.5 to 10 mol, preferably from 0.8 to 2 mol, of amine of the formula (III) are employed per mole of halopyrazolopyrimidine of the formula (II). Work-up is carried out by customary methods.

The formula (Ia) provides a general definition of the pyrazolopyrimidines required as starting materials for carrying out the process (b) according to the invention. In this formula, R¹, R², R³ and X¹ preferably have those meanings which have already been mentioned in connection with the description of the subtances of the formula (I) according to the invention as being preferred for these radicals.

The pyrazolopyrimidines of the formula (Ia) are substances according to the invention which can be prepared by the process (a) according to the invention.

Suitable diluents for carrying out the process (b) according to the invention are all customary inert organic solvents. Preference is given to using aliphatic or aromatic, optionally halogenated hydrocarbons, such as toluene, dichloromethane, chloroform or carbon tetrachloride.

When carrying out the process (b) according to the invention, the reaction temperatures can be varied within a certain range. In general, the process is carried out at temperatures between −80° C. and +20° C., preferably between −60° C. and +10° C.

In general, the process (b) according to the invention is carried out under atmospheric pressure. However, it is also possible to operate under elevated pressure.

When carrying out the process (b) according to the invention, in general an equivalent amount or else an excess, preferably from 1.1 to 1.2 mol, of diisobutylaluminium hydride are employed per mole of pyrazolopyrimidine of the formula (Ia), and an excess of aqueous ammonium chloride solution is then added. Work-up is carried out by customary methods. In general, the reaction mixture is acidified, the organic phase is separated off, the aqueous phase is extracted with an organic solvent which is poorly water-miscible and the combined organic phases are washed, dried and concentrated under reduced pressure.

The formula (Ib) provides a general definition of the pyrazolopyrimidines required as starting materials for carrying out the process (c) according to the invention. In this formula, R¹, R², R³ and X¹ preferably have those meanings which have already been mentioned in connection with the description of the substances of the formula (I) according to the invention as being preferred for these radicals.

The pyrazolopyrimidines of the formula (Ib) are substances according to the invention which can be prepared by the process (b) according to the invention.

The formula (IV) provides a general definition of the amino compounds required as reaction components for carrying out the process (c) according to the invention. In this formula, R⁴ preferably represents hydrogen or alkyl having 1 to 4 carbon atoms, particularly preferably hydrogen, methyl or ethyl.

Suitable reaction components include acid addition salts, preferably hydrogen chloride addition salts of amino compounds of the formula (IV).

Both the amino compounds of the formula (IV) and their acid addition salts are known or can be prepared by known methods.

Suitable diluents for carrying out the process (c) according to the invention are all customary inert organic solvents. Preference is given to using alcohols, such as methanol, ethanol, n-propanol or isopropanol.

Suitable catalysts for carrying out the process (c) according to the invention are all reaction accelerators customary for such reactions. Preference is given to using acidic or basic catalysts, such as, for example, weak basic ion exchangers commercially available under the name Amberlyst A-21®.

When carrying out the process (c) according to the invention, the reaction temperatures can be varied within a certain range. In general, the process is carried out at temperatures between 0° C. and 80° C., preferably between 10° C. and 60° C.

In general, the process (c) according to the invention is carried out under atmospheric pressure. However, it is also possible to operate under elevated pressure.

When carrying out the process (c) according to the invention, in general an equivalent amount or an excess, preferably between 1.1 and 1.5 mol, of the amino compound of the formula (IV) or an acid addition salt thereof is employed per mole of pyrazolopyrimidine of the formula (Ib). Work-up is carried out by customary methods. In general, the reaction mixture is, if required, filtered and then concentrated and purified.

Preferred acids for preparing acid addition salts of pyrazolopyrimidines of the formula (I) are those acids which have already been mentioned as preferred acids in connection with the description of the acid addition salts according to the invention.

The acid addition salts of the compounds of the formula (I) can be obtained in a simple manner by customary methods for forming salts, for example by dissolving a compound of the formula (I) in a suitable inert solvent and adding the acid, for example hydrochloric acid, and they can be isolated in a known manner, for example by filtering off and, if appropriate, be purified by washing with an inert organic solvent.

The active compounds according to the invention are suitable for controlling animal pests, in particular insects, arachnids and nematodes, which are encountered in agriculture, in forestry, in the protection of stored products and of materials, and in the hygiene sector, and have good plant tolerance and favourable toxicity to warm-blooded animals. They may preferably be employed as plant protection agents. They are active against normally sensitive and resistant species and against all or some stages of development. The abovementioned pests include:

From the order of the Isopoda, for example, Oniscus asellus, Armadillidium vulgare and Porcellio scaber.

From the order of the Diplopoda, for example, Blaniulus guttulatus.

From the order of the Chilopoda, for example, Geophilus carpophagus and Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Acheta domesticus, Gryllotalpa spp., Locusta migratoria migratorioides, Melanoplus spp. and Schistocerca gregaria.

From the order of the Blattaria, for example, Blatta orientalis, Periplaneta americana, Leucophaea maderae, Blattella germanica.

From the order of the Dermaptera, for example, Forficula auricularia.

From the order of the Isoptera, for example, Reticulitermes spp.

From the order of the Phthiraptera, for example, Pediculus humanus corporis, Haematopinus spp., Linognathus spp., Trichodectes spp. and Damalinia spp.

From the order of the Thysanoptera, for example, Hercinothrips femoralis, Thrips tabaci, Thrips palmi and Frankliniella occidentalis.

From the order of the Heteroptera, for example, Eurygaster spp., Dysdercus intermedius, Piesma quadrata, Cimex lectularius, Rhodnius prolixus and Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium comi, Saissetia oleae, Laodelphax striatellus, Nilaparvata lugens, Aonidiella aurantii, Aspidiotus hederae, Pseudococcus spp. and Psylla spp.

From the order of the Lepidoptera, for example, Pectinophora gossypiella, Bupalus piniarius, Cheimatobia brumata, Lithocolletis blancardella, Hyponomeuta padelia, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pornella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp., Oulema oryzae.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica and Lissorhoptrus oryzophilus.

From the order of the Hymenoptera, for example, Diprion spp., Hoplocampa spp., Lasius spp., Monomorium pharaonis and Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp. and Liriomyza spp.

From the order of the Siphonaptera, for example, Xenopsylla cheopis and Ceratophyllus spp.

From the class of the Arachnida, for example, Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp., Brevipalpus spp.

The phytoparasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp., Bursaphelenchus spp.

The active compounds can be used with particularly good results for controlling plant-damaging insects, such as, for example, against the caterpillars of the diamondback moth (Plutella maculipennis).

The substances according to the invention also have potent microbicidal activity and can be employed for controlling undesirable microorganisms, such as fungi and bacteria, in crop protection and in the protection of materials.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Some pathogens causing fungal and bacterial diseases which come under the generic names listed above may be mentioned as examples, but not by way of limitation:

Xanthomonas species, such as, for example, Xanthomonas campestris pv. oryzae;

Pseudomonas species, such as, for example, Pseudomonas syringae pv. lachrymans;

Erwinia species, such as, for example, Erwinia amylovora;

Pythium species, such as, for example, Pythium ultimum;

Phytophthora species, such as, for example, Phytophthora infestans;

Pseudoperonospora species, such as, for example, Pseudoperonospora humuli or Pseudoperonospora cubensis;

Plasmopara species, such as, for example, Plasmopara viticola;

Bremia species, such as, for example, Bremia lactucae;

Peronospora species, such as, for example, Peronospora pisi or P. brassicae;

Erysiphe species, such as, for example, Erysiphe graminis;

Sphaerotheca species, such as, for example, Sphaerotheca fuliginea;

Podosphaera species, such as, for example, Podosphaera leucotricha;

Venturia species, such as, for example, Venturia inaequalis;

Pyrenophora species, such as, for example, Pyrenophora teres or P. graminea (conidia form: Drechslera, syn: Helminthosporium);

Cochliobolus species, such as, for example, Cochliobolus sativus (conidia form: Drechslera, syn: Helminthosporium);

Uromyces species, such as, for example, Uromyces appendiculatus;

Puccinia species, such as, for example, Puccinia recondita;

Sclerotinia species, such as, for example, Sclerotinia sclerotiorum;

Tilletia species, such as, for example, Tilletia caries;

Ustilago species, such as, for example, Ustilago nuda or Ustilago avenae;

Pellicularia species, such as, for example, Pellicularia sasakii;

Pyricularia species, such as, for example, Pyricularia oryzae;

Fusarium species, such as, for example, Fusarium culmorum;

Botrytis species, such as, for example, Botrytis cinerea;

Septoria species, such as, for example, Septoria nodorum;

Leptosphaeria species, such as, for example, Leptosphaeria nodorum;

Cercospora species, such as, for example, Cercospora canescens;

Alternaria species, such as, for example, Alternaria brassicae; and

Pseudocercosporella species, such as, for example, Pseudocercosporella herpotrichoides.

The active compounds according to the invention also have very good fortifying action in plants. Accordingly, they can be used for mobilizing the defences of the plant against attack by unwanted microorganisms.

In the present context, plant-fortifying (resistance-inducing) substances are to be understood as meaning those substances which are capable of stimulating the defence system of plants such that, when the treated plants are subsequently inoculated with unwanted microorganisms, they show substantial resistance against these mircroorganisms.

In the present case, unwanted microorganisms are to be understood as meaning phytopathogenic fungi, bacteria and viruses. Accordingly, the substances according to the invention can be used to protect plants for a certain period after the treatment against attack by the pathogens mentioned. The period for which protection is provided generally extends over 1 to 10 days, preferably 1 to 7 days, after the treatment of the plants with the active compounds.

The fact that the active compounds according to the invention are well tolerated by plants at the concentrations required for controlling plant diseases permits the treatment of above-ground parts of plants, of propagation stock and seeds, and of the soil.

The active compounds according to the invention can be used with particularly good results for controlling cereal diseases, such as, for example, against Fusarium species, diseases in viticulture and fruit and vegetable growing, such as, for example, against Botrytis, Venturia and Alternaria species, or rice diseases, such as, for example, against Pyricularia species.

The active compounds according to the invention are also suitable for increasing the yield of crops. In addition, they show reduced toxicity and are well tolerated by plants.

If appropriate, the compounds according to the invention can, at certain concentrations and application rates, also be used as herbicides and for influencing plant growth. If appropriate they can also be employed as intermediates and precursors for the synthesis of other active compounds.

Plants and plant parts can be treated with the active compounds according to the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and recombinant methods or by combinations of these methods, including the transgenic plants and inclusive of the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material for example cuttings, tubers, rhizomes, offsets and seeds.

Treatment of the plants and plant parts with the active compounds according to the invention is carried out directly or by allowing the compounds to act on their surroundings, environment or storage space by the customary treatment methods, for example by immersion, spraying, evaporation, fogging, scattering, painting on and, in the case of propagation material, in particular in the case of seeds, also by applying one or more coats.

In the protection of materials, the compounds according to the invention can be employed for protecting industrial materials against infection with and destruction by undesired microorganisms.

Industrial materials in the present context are understood as meaning non-living materials which have been prepared for use in industry. For example, industrial materials which are intended to be protected by active compounds according to the invention from microbial change or destruction can be adhesives, sizes, paper and board, textiles, leather, wood, paints and plastic articles, cooling lubricants and other materials which can be infected with or destroyed by microorganisms. Parts of production plants, for example cooling-water circuits, which may be impaired by the proliferation of microorganisms may also be mentioned within the scope of the materials to be protected. Industrial materials which may be mentioned within the scope of the present invention are preferably adhesives, sizes, paper and board, leather, wood, paints, cooling lubricants and heat-transfer liquids, particularly preferably wood.

Microorganisms capable of degrading or changing the industrial materials which may be mentioned are, for example, bacteria, fungi, yeasts, algae and slime organisms. The active compounds according to the invention preferably act against fungi, in particular moulds, wood-discolouring and wood-destroying fungi (Basidiomycetes), and against slime organisms and algae.

Microorganisms of the following genera may be mentioned as examples:

Alternaria, such as Altemaria tenuis,

Aspergillus, such as Aspergillus niger,

Chaetomium, such as Chaetomium globosum,

Coniophora, such as Coniophora puetana,

Lentinus, such as Lentinus tigrinus,

Penicillium, such as Penicillium glaucum,

Polyporus, such as Polyporus versicolor,

Aureobasidium, such as Aureobasidium pullulans,

Sclerophoma, such as Sclerophoma pityophila,

Trichoderma, such as Trichoderma viride,

Escherichia, such as Escherichia coli,

Pseudomonas, such as Pseudomonas aeruginosa, and

Staphylococcus, such as Staphylococcus aureus.

Depending on their particular physical and/or chemical properties, the active compounds can be converted into the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is, liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as finely divided silica, alumina and silicates. Suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates. Suitable dispersants are: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95% by weight of active compound, preferably between 0.5 and 90%.

The active compounds according to the invention can be used as such or in their formulations, also in a mixture with known fungicides, bactericides, acaricides, nematicides or insecticides, to broaden, for example, the activity spectrum or to prevent development of resistance. In many cases, synergistic effects are obtained, i.e. the activity of the mixture is greater than the activity of the individual components.

Examples of suitable mixing components are the following:

Fungicides:

aldimorph, ampropylfos, ampropylfos-potassium, andoprim, anilazine, azaconazole, azoxystrobin,

benalaxyl, benodanil, benomyl, benzamacril, benzamacril-isobutyl, bialaphos, binapacryl, biphenyl, bitertanol, blasticidin-S, bromuconazole, bupirimate, buthiobate,

calcium polysulphide, carpropamid, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram,

debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon,

edifenphos, epoxiconazole, etaconazole, ethirimol, etridiazole,

famoxadon, fenapanil, fenarimol, fenbuconazole, fenfuram, fenhexamid, fenitropan, fenpiclonil, fenpropidin, fenpropimorph, fentin acetate, fentin hydroxide, ferbam, ferimzone, fluazinam, flumetover, fluoromide, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox, fluoxastrobin,

guazatine,

hexachlorobenzene, hexaconazole, hymexazole,

imazalil, imibenconazole, iminoctadine, iminoctadine albesilate, iminoctadine triacetate, iodocarb, ipconazole, iprobenfos (IBP), iprodione, iprovalicarb, irumamycin, isoprothiolane, isovaledione,

kasugamycin, kresoxim-methyl, copper preparations, such as: copper hydroxide, copper naphthenate, copper oxychloride, copper sulphate, copper oxide, oxine-copper and Bordeaux mixture,

mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin,

nickel dimethyldithiocarbamate, nitrothal-isopropyl, nuarimol,

ofurace, oxadixyl, oxamocarb, oxolinic acid, oxycarboxim, oxyfenthiin,

paclobutrazole, pefurazoate, penconazole, pencycuron, phosdiphen, picoxystrobin, pimaricin, piperalin, polyoxin, polyoxorim, probenazole, prochloraz, procymidone, propamocarb, propanosine-sodium, propiconazole, propineb, pyraclostrobin, pyrazophos, pyrifenox, pyrimethanil, pyroquilon, pyroxyfur, prothioconazole,

quinconazole, quintozene (PCNB), quinoxyfen,

sulphur and sulphur preparations, spiroxamine,

tebuconazole, tecloftalam, tecnazene, tetcyclacis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, tricyclazole, tridemorph, trifloxystrobin, triflumizole, triforine, triticonazole,

uniconazole,

validamycin A, vinclozolin, viniconazole,

zarilamide, zineb, ziram and also

Dagger G,

OK-8705,

OK-8801,

α-(1,1-dimethylethyl)-β-(2-phenoxyethyl)-1H-1,2,4-triazole-1-ethanol,

α-(2,4-dichlorophenyl)-β-fluoro-β-propyl-1H-1,2,4-triazole-1-ethanol,

α-(2,4-dichlorophenyl)-α-methoxy-α-methyl-1H-1,2,4-triazole-1-ethanol,

α-(5-methyl-1,3-dioxan-5-yl)-β-[[4-(trifluoromethyl)phenyl]methylene]-1H-1,2,4-triazole-1-ethanol,

(5RS ,6RS)-6-hydroxy-2,2,7,7-tetramethyl-5-(1H-1,2,4-triazol-1-yl)-3-octanone,

(E)-α-(methoxyimino)-N-methyl-2-phenoxyphenylacetamide,

1-(2,4-dichlorophenyl)-2-(1H-1,2,4-triazol-1-yl)ethanone O-(phenylmethyl)oxime,

1-(2-methyl-1-naphthalenyl)-1H-pyrrole-2,5-dione,

1-(3,5-dichlorophenyl)-3-(2-propenyl)-2,5-pyrrolidinedione,

1-[(diiodomethyl)sulphonyl]-4-methylbenzene,

1-[[2-(2,4-dichlorophenyl)-1,3-dioxolan-2-yl]methyl]-1H-imidazole,

1-[[2-(4-chlorophenyl)-3-phenyloxiranyl]methyl]-1H-1,2,4-triazole,

1-[1-[2-[(2,4-dichlorophenyl)methoxy]phenyl]ethenyl]-1H-imidazole,

1-methyl-5-nonyl-2-(phenylmethyl)-3-pyrrolidinole,

2′,6′-dibromo-2-methyl-4′-trifluoromethoxy-4′-trifluoromethyl-1,3-thiazole-5-carboxanilide,

2,6-dichloro-5-(methylthio)-4-pyrimidinylthiocyanate,

2,6-dichloro-N-(4-trifluoromethylbenzyl)benzamide,

2,6-dichloro-N-[[4-(trifluoromethyl)phenyl]methyl]benzamide,

2-(2,3,3-triiodo-2-propenyl )-2H-tetrazole,

2-[(1-methylethyl)sulphonyl]-5-(trichloromethyl)-1,3,4-thiadiazole,

2-[[6-deoxy-4-O-(4-O-methyl-β-D-glycopyranosyl])-α-D-glucopyranosyl]amino]-4-methoxy-1H-pyrrolo[2,3-d]pyrimidine-5-carbonitrile,

2-aminobutane,

2-bromo-2-(bromomethyl)pentanedinitrile,

2-chloro-N-(2,3-dihydro-1,1,3-trimethyl-1H-inden-4-yl)-3-pyridinecarboxamide,

2-chloro-N-(2,6-dimethylphenyl)-N-(isothiocyanatomethyl)acetamide,

2-phenylphenol (OPP),

3,4-dichloro-1-[4-(difluoromethoxy)phenyl]-1H-pyrrole-2,5-dione,

3,5-dichloro-N-[cyano[(1-methyl-2-propynyl)oxy]methyl]benzamide,

3-(1,1-dimethylpropyl-1-oxo-1H-indene-2-carbonitrile,

3-[2-(4-chlorophenyl)-5-ethoxy-3-isoxazolidinyl]pyridine,

4-chloro-2-cyano-N,N-dimethyl-5-(4-methylphenyl)-1H-imidazole-1-sulphonamide,

4-methyltetrazolo[1,5-a]quinazolin-5(4H)-one,

8-hydroxyquinoline sulphate,

9H-xanthene-2-[(phenylamino)carbonyl]-9-carboxylic hydrazide,

bis-(1-methylethyl)-3-methyl-4-[(3-methylbenzoyl)oxy]-2,5-thiophenedicarboxylate,

cis-1-(4-chlorophenyl)-2-(1H-1,2,4-triazol-1-yl)cycloheptanol,

cis-4-[3-[4-(1,1-dimethylpropyl)-phenyl-2-methylpropyl]-2,6-dimethylmorpholine-hydrochloride,

ethyl[(4-chlorophenyl)azo]cyanoacetate,

potassium hydrogen carbonate,

methanetetrathiol sodium salt,

methyl 1-(2,3-dihydro-2,2-dimethyl-1H-inden-1-yl)-1H-imidazole-5-carboxylate,

methyl N-(2,6-dimethylphenyl)-N-(5-isoxazolylcarbonyl)-DL-alaninate,

methyl N-(chloroacetyl)-N-(2,6-dimethylphenyl)-DL-alaninate,

N-(2,6-dimethylphenyl)-2-methoxy-N-(tetrahydro-2-oxo-3-furanyl)acetamide,

N-(2,6-dimethylphenyl)-2-methoxy-N-(tetrahydro-2-oxo-3-thienyl)acetamide,

N-(2-chloro-4-nitrophenyl)-4-methyl-3-nitrobenzenesulphonamide,

N-(4-cyclohexylphenyl)-1,4,5,6-tetrahydro-2-pyrimidineamine,

N-(4-hexylphenyl)-1,4,5,6-tetrahydro-2-pyrimidineamine,

N-(5-chloro-2-methylphenyl)-2-methoxy-N-(2-oxo-3-oxazolidinyl)acetamide,

N-(6-methoxy-3-pyridinyl)cyclopropanecarboxamide,

N-[2,2,2-trichloro-1-[(chloroacetyl)amino]ethyl]benzamide,

N-[3-choro-4,5-bis-(2-propinyloxy)phenyl]-N′-methoxymethaneimidamide,

N-formyl-N-hydroxy-DL-alanine sodium salt,

O,O-diethyl[2-(dipropyamino)-2-oxoethyl]ethylphosphoramidothioate,

O-methyl S-phenyl phenylpropylphosphoramidothioate,

S-methyl 1,2,3-benzothiadiazole-7-carbothioate,

spiro[2H]-1-benzopyrane-2,1′(3′H)-isobenzofuran]-3′-one,

4-[3,4-dimethoxyphenyl)-3-(4-fluorophenyl)acryloyl]morpholine.

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

Insecticides/Acaricides/Nematicides:

abamectin, acephate, acetamiprid, acrinathrin, alanycarb, aldicarb, aldoxycarb, alpha-cypermethrin, alphamethrin, amitraz, avermectin, AZ 60541, azadirachtin, azamethiphos, azinphos A, azinphos M, azocyclotin,

Bacillus popilliae, Bacillus sphaericus, Bacillus subtilis, Bacillus thuringiensis, baculoviruses, Beauveria bassiana, Beauveria tenella, bendiocarb, benfuracarb, bensultap, benzoximate, betacyfluthrin, bifenazate, bifenthrin, bioethanomethrin, biopermethrin, bistrifluron, BPMC, bromophos A, bufencarb, buprofezin, butathiofos, butocarboxim, butylpyridaben,

cadusafos, carbaryl, carbofuran, carbophenothion, carbosulfan, cartap, chloethocarb, chlorethoxyfos, chlorfenapyr, chlorfenvinphos, chlorfluazuron, chlormephos, chlorpyrifos, chlorpyrifos M, chlovaporthrin, chromafenozide, cis-resmethrin, cispermethrin, clocythrin, cloethocarb, clofentezine, clothianidine, cyanophos, cycloprene, cycloprothrin, cyfluthrin, cyhalothrin, cyhexatin, cypermethrin, cyromazine,

deltamethrin, demeton M, demeton S, demeton-S-methyl, diafenthiuron, diazinon, dichlorvos, dicofol, diflubenzuron, dimethoate, dimethylvinphos, diofenolan, disulfoton, docusat-sodium, dofenapyn,

eflusilanate, emamectin, empenthrin, endosulfan, Entomopfthora spp., esfenvalerate, ethiofencarb, ethion, ethoprophos, etofenprox, etoxazole, etrimfos,

fenamiphos, fenazaquin, fenbutatin oxide, fenitrothion, fenothiocarb, fenoxacrim, fenoxycarb, fenpropathrin, fenpyrad, fenpyrithrin, fenpyroximate, fenvalerate, fipronil, fluazinam, fluazuron, flubrocythrinate, flucycloxuron, flucythrinate, flufenoxuron, flumethrin, flutenzine, fluvalinate, fonophos, fosmethilan, fosthiazate, fubfenprox, furathiocarb,

granulosis viruses,

halofenozide, HCH, heptenophos, hexaflumuron, hexythiazox, hydroprene,

imidacloprid, indoxacarb, isazofos, isofenphos, isoxathion, ivermectin,

nuclear polyhedrosis viruses,

lambda-cyhalothrin, lufenuron,

malathion, mecarbam, metaldehyde, methamidophos, Metharhizium anisopliae, Metharhizium flavoviride, methidathion, methiocarb, methoprene, methomyl, methoxyfenozide, metolcarb, metoxadiazone, mevinphos, milbemectin, milbemycin, monocrotophos,

naled, nitenpyram, nithiazine, novaluron,

omethoate, oxamyl, oxydemethon M,

Paecilomyces fumosoroseus, parathion A, parathion M, permethrin, phenthoate, phorat, phosalone, phosmet, phosphamidon, phoxim, pirimicarb, pirimiphos A, pirimiphos M, profenofos, promecarb, propargite, propoxur, prothiofos, prothoat, pymetrozine, pyraclofos, pyresmethrin, pyrethrum, pyridaben, pyridathion, pyrimidifen, pyriproxyfen,

quinalphos,

ribavirin,

salithion, sebufos, silafluofen, spinosad, spirodiclofen, sulfotep, sulprofos,

tau-fluvalinate, tebufenozide, tebufenpyrad, tebupirimiphos, teflubenzuron, tefluthrin, temephos, temivinphos, terbufos, tetrachlorvinphos, tetradifon theta-cypermethrin, thiacloprid, thiamethoxam, thiapronil, thiatriphos, thiocyclam hydrogen oxalate, thiodicarb, thiofanox, thuringiensin, tralocythrin, tralomethrin, triarathene, triazamate, triazophos, triazuron, trichlophenidine, trichlorfon, triflumuron, trimethacarb,

vamidothion, vaniliprole, Verticillium lecanii,

YI 5302

zeta-cypermethrin, zolaprofos

(1R-cis)-[5-(phenylmethyl)-3-furanyl]methyl 3-[(dihydro-2-oxo-3(2H)-furanylidene)methyl]-2,2-dimethylcyclopropanecarboxylate,

(3-phenoxyphenyl)methyl 2,2,3,3-tetramethylcyclopropanecarboxylate,

1-[(2-chloro-5-thiazolyl)methyl]tetrahydro-3,5-dimethyl-N-nitro-1,3,5-triazine-2(1H)-imine,

2-(2-chloro-6-fluorophenyl)-4-[4-(1,1-dimethylethyl)phenyl]-4,5-dihydrooxazole,

2-(acetyloxy)-3-dodecyl-1,4-naphthalenedione,

2-chloro-N-[[[4-(1-phenylethoxy)phenyl]amino]carbonyl]benzamide,

2-chloro-N-[[[4-(2,2-dichloro-1,1-difluoroethoxy)phenyl]amino]carbonyl]benz-amide,

3-methylphenyl propylcarbamate,

4-[4-(4-ethoxyphenyl)-4-methylpentyl]-1-fluoro-2-phenoxybenzene,

4-chloro-2-(1,1-dimethylethyl)-5-[[2-(2,6-dimethyl-4-phenoxyphenoxy)ethyl]thio]-3(2H)-pyridazinone,

4-chloro-2-(2-chloro-2-methylpropyl)-5-[(6-iodo-3-pyridinyl)methoxy]-3(2H)-pyridazinone,

4-chloro-5-[(6-chloro-3-pyridinyl)methoxy]-2-(3,4-dichlorophenyl)-3(2H)-pyridazinone,

Bacillus thuringiensis strain EG-2348,

[2-benzoyl-1-(1,1-dimethylethyl)-hydrazidobenzoic acid,

2,2-dimethyl-3-(2,4-dichlorophenyl)-2-oxo-1-oxaspiro[4.5]dec-3-en-4-yl butanoate,

[3-[(6-chloro-3-pyridinyl)methyl]-2-thiazolidinylidene]cyanamide,

dihydro-2-(nitromethylene)-2H-1,3-thiazine-3(4H)carboxaldehyde,

ethyl[2-[[1,6-dihydro-6-oxo-1-(phenylmethyl)-4-pyridazinyl]oxy]ethyl]carbamate,

N-(3,4,4-trifluoro-1-oxo-3-butenyl)glycine,

N-(4-chlorophenyl)-3-[4-(difluoromethoxy)phenyl]-4,5-dihydro-4-phenyl-1H-pyrazole-1-carboxamide,

N-[(2-chloro-5-thiazolyl)methyl]-N′-methyl-N″-nitroguanidine,

N-methyl-N′-(1-methyl-2-propenyl)-1,2-hydrazinedicarbothioamide,

N-methyl-N′-2-propenyl-1,2-hydrazinedicarbothioamide,

O,O-diethyl[2-(dipropylamino)-2-oxoethyl]ethylphosphoramidothioate,

N-cyanomethyl-4-trifluoromethylnicotinamide,

3,5-dichloro-1-(3,3-dichloro-2-propenyloxy)-4-[3-(5-trifluoromethylpyridin-2-yloxy)propoxy]benzene.

A mixture with other known active compounds, such as herbicides, or with fertilizers and growth regulators, is also possible.

In addition, the compounds of the formula (I) according to the invention also have very good antimycotic activity. They have a very broad antimycotic activity spectrum in particular against dermatophytes and yeasts, moulds and diphasic fungi (for example against Candida species, such as Candida albicans, Candida glabrata), and Epidermophyton floccosum, Aspergillus species, such as Aspergillus niger and Aspergillus fumigatus, Trichophyton species, such as Trichophyton mentagrophytes, Microsporon species such as Microsporon canis and audouinii. The list of these fungi by no means limits the mycotic spectrum covered, but is only for illustration.

The active compounds can be used as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, spreading, etc. It is furthermore possible to apply the active compounds by the ultra-low-volume method, or to inject the active compound preparation or the active compound itself into the soil. It is also possible to treat the seeds of the plants.

When using the active compounds according to the invention as fungicides, the application rates can be varied within a relatively wide range, depending on the kind of application. For the treatment of parts of plants, the active compound application rates are generally between 0.1 and 10,000 g/ha, preferably between 10 and 1000 g/ha. For seed dressing, the active compound application rates are generally between 0.001 and 50 g per kilogram of seed, preferably between 0.01 and 10 g per kilogram of seed. For the treatment of the soil, the active compound application rates are generally between 0.1 and 10,000 g/ha, preferably between 1 and 5000 g/ha.

When used as insecticides, the active compounds according to the invention can furthermore be present in their commercially available formulations and in the use forms, prepared from these formulations, as a mixture with synergistic agents. Synergistic agents are compounds which increase the action of the active compounds, without it being necessary for the synergistic agent added to be active itself.

The active compound content of the use forms prepared from the commercially available formulations can vary within wide limits. The active compound concentration of the use forms can be from 0.0000001 to 95% by weight of active compound, preferably between 0.0001 and 1% by weight.

The compounds are employed in a customary manner appropriate for the use forms.

When used against hygiene pests and pests of stored products, the active compound is distinguished by an excellent residual action on wood and clay as well as by a good stability to alkali on limed substrates.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the plant cultivars which are in each case commercially available or in use are treated according to the invention. Plant cultivars are understood as meaning plants with novel properties (“traits”) which are grown by conventional cultivation, by mutagenesis or by recombinant DNA techniques. These may be cultivars, biotypes or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions to be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which are actually to be expected.

The transgenic plants or plant cultivars (i.e. those obtained by genetic engineering) which are preferably to be treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapes), and particular emphasis is given to maize, soya beans, potatoes, cotton and oilseed rape. Traits that are emphasized are in particular increased defence of the plants against insects by toxins formed in the plants, in particular those formed in the plants by the genetic material from Bacillus thuringiensis (for example by the genes CryIA(a), CryIA(b), CryIA(c), CrylIA, CryIIIA, CryIIIB2, Cry9c Cry2Ab, Cry3Bb and CryIF and also combinations thereof) (hereinbelow referred to as “Bt plants”). Traits which are also particularly emphasized are the increased resistance of plants to fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which cultivars will be developed and/or marketed in the future.

The plants listed can be treated according to the invention in a particularly advantageous manner with the compounds of the formula I or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The invention is illustrated by the following examples.

### PREPARATION EXAMPLES

### Examples 1 and 2

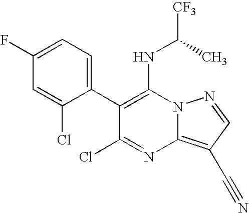

2.5 g (7.3 mmol) of 3-cyano-5,7-dichloro-6-(2-chloro-4-fluorophenyl)pyrazolo[1,5-a]pyrimidine and 0.425 g (7.3 mmol) of potassium fluoride in 7.8 g of acetonitrile are stirred at 60° C. for 3 hours. 3.31 g (29.3 mmol) of (S)-1,1,1-trifluoroprop-2-ylamine are then added, and the mixture is stirred at 80° C. for another 15 hours. The solvent is distilled off under reduced pressure and the residue is treated with dichloromethane and 1 N aqueous hydrochloric acid. The organic phase is separated off and dried over sodium sulphate, and the solvent is distilled off under reduced pressure. The residue is chromatographed on silica gel using a mixture of 4 parts of cyclohexane and 1 part of ethyl acetate. Two different product fractions (fraction 1 and fraction 2) are isolated.

Fraction 1 (1.2 g) is chromatographed again on silica gel using a mixture of 9 parts of n-hexane and 1 part of acetone. This gives 0.8 g (21% of theory) of 3-cyano-5-chloro-6-(2-chloro-4-fluorophenyl)-7-(S)-1′,1′,1′-trifluoroprop-2-ylaminopyrazolo[1,5-a]pyrimidine as atropisomer A (Example 1) (purity: 80.4%)

HPLC: log P=3.88 (isomer AS)

¹H-NMR (DMSO-d6, tetramethylsilane): δ=1.37, 1.38 (3H); 4.88, 4.90 (1H); 7.43-7.59 (1H); 7.60-7.66 (1H); 7.72-7.78 (1H); 8.06, 8.08 (1H, NH); 8.83 (1H) ppm.

Fraction 2, isolated last, contains 0.9 g (29.3% of theory) of 3-cyano-5-chloro-6-(2-chloro-4-fluorophenyl)-7-(S)-1′,1′,1′-trifluoroprop-2-ylaminopyrazolo[1,5-a]pyrimidine as atropisomer B (Example 2) (purity: 99.3%)

HPLC: log P=3.91 (isomer BS)

¹H-NMR (DMSO-d6, tetramethylsilane): δ=1.29, 1.31 (3H); 4.61, 4.63 (1H); 7.42-7.47 (1H); 7.58-7.61 (1H); 7.73-7.76 (1H); 8.10, 8.12 (1H, NH); 8.84 (1H) ppm.

### Example 3

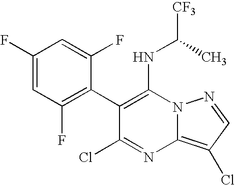

0.165 g (9.75 g, 237.5 mmol) of potassium fluoride and 0.481 g (4.26 mmol) of (S)-1,1,1-trifluoroprop-2-ylamine are added to a solution of 0.5 g (1.4 mmol) of 3,5,7-trichloro-6-(2,4,6-trifluorophenyl)pyrazolo[1,5-a]pyrimidine in 12.5 ml of acetonitrile, and the mixture is stirred at 80° C. for 16 hours. After cooling, 1N of hydrochloric acid and dichlormethane are added. The reaction mixture is filtered and the filtrate is concentrated. The residue is chromatographed on a silica gel cartridge using methyl t-butyl ether/petroleum ether (1:100). This gives 0.25 g (40% of theory) of N-[3,5-dichloro-6-(2,4,6-trifluorophenyl)pyrazolo[1,5-a]pyrimidin-7-yl]-N-[(1S)-2,2,2-trifluoro-1-methylethyl]amine.

HPLC; log P=4.43

### Example 4

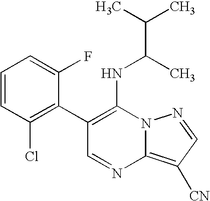

0.1 g (0.33 mmol) of 7-chloro-6-(2-chloro-6-fluorophenyl)pyrazolo[1,5-a]pyrimidine-3-carbonitrile and 0.028 g (0.33 mmol) of 1,2-dimethylpropylamine are dissolved in 5 ml of dichloromethane. 0.05 ml of triethylamine is added, and the reaction mixture is stirred at room temperature for 16 hours. The reaction mixture is stirred with 1N hydrochloric acid and then filtered, and the filtrate is concentrated under reduced pressure. The residue is chromatographed on a silica gel cartridge using methyl t-butyl ether/petroleum ether (1:9). This gives 0.1 g (89% of theory) of 6-(2-chloro-6-fluorophenyl)-7-[(1,2-dimethylpropyl)amino)pyrazolo[1,5-a]pyrimidine-3-carbonitrile.

HPLC; log P=3.78

### Example 5

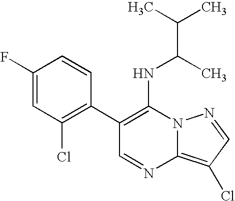

0.1 g (0.316 mmol) of 7-chloro-6-(2-chloro-6-fluorophenyl)pyrazolo[1,5-a]pyrimidine-3-carbonitrile and 0.028 g (0.316 mmol) of 1,2-dimethylpropylamine are dissolved in 4 ml of acetonitrile. 0.044 g (0.316 mmol) of potassium carbonate is added, and the reaction mixture is stirred at 60° C. for 16 hours. 20 ml of ether and 10 ml of 1N hydrochloric acid are added to the reaction mixture. The organic phase is separated off, dried over sodium sulphate and concentrated under reduced pressure.

The residue is chromatographed on a silica gel cartridge using methyl t-butyl ether/petroleum ether (1:30). This gives 0.08 g (67% of theory) of N-[3-chloro-6-(2-chloro-4-fluorophenyl)pyrazolo[1,5-a]pyrimidin-7-yl]-N-(1,2-dimethylpropyl)amine.

HPLC; log P=4.53

The compounds of the formula

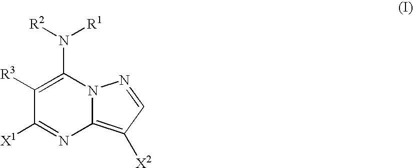

listed in Table 1 below are also prepared by the methods described above.

TABLE 1

| Ex. No. | R¹ | R² | R³ | X¹ | X² | Isomer** | logP | (° C.) |
|---|---|---|---|---|---|---|---|---|
| | | | | | | | | m.p.: |
| 6 | —CH₂—C(CH₃)═CH₂ | —C₂H₅ | 2,4,6-trifluorophenyl | —Cl | —CN | | 4.6 | |
| 7 | 2,2,2-trifluoro-1-methylethyl | —H | 2,4,6-trifluorophenyl | —Cl | —CN | S | 3.69 | |
| 8 | —CH₂—C(CH₃)═CH₂ | —C₂H₅ | 2-fluorophenyl | —Cl | —CN | | 4.38 | |
| 9 | 2-methoxyethyl | —C₂H₅ | 2-fluorophenyl | —Cl | —CN | | 3.52 | |
| 10 | cyclopentyl | —H | 2-fluorophenyl | —Cl | —CN | | 3.89 | |
| | | | | | | | | Fp.: |
| 11 | cyclopropylmethyl | —H | 2-fluorophenyl | —Cl | —CN | | 3.47 | |
| 12 | 2,2,2-trifluoro-1-methylethyl | —H | 2-chloro-6-fluorophenyl | —Cl | —CN | S | 3.73 | |
| 13 | —CH₂—C(CH₃)═CH₂ | —C₂H₅ | 2-chloro-6-fluorophenyl | —Cl | —CN | | 4.68 | |
| 14 | —CH₂—CH₂—O—CH₂—CH₂— | * | 2-chloro-6-fluorophenyl | —Cl | —CN | | 3.26 | |
| 15 | n-butyl | —C₂H₅ | 2-chloro-6-fluorophenyl | —Cl | —CN | | 4.92 | |
| 16 | i-butyl | —H | 2-chloro-6-fluorophenyl | —Cl | —CN | | 3.94 | |
| 17 | —CH₂—C(CH₃)₃ | —H | 2-chloro-6-fluorophenyl | —Cl | —CN | | 4.41 | |
| 18 | —CH₂—C(CH₃)═CH₂ | —H | 2-chloro-6-fluorophenyl | —Cl | —CN | | 3.65 | |
| 19 | —CH₂—CH₂—CH₂—CH₂— | * | 2-chloro-6-fluorophenyl | —Cl | —CN | | 3.82 | |
| 20 | —C₂H₅ | —C₂H₅ | 2-chloro-6-fluorophenyl | —Cl | —CN | | 4.13 | |
| 21 | —CH₂—CH—CH₂—CH₂—CH₂— | * | 2-chloro-6-fluorophenyl | —Cl | —CN | | 4.32 | |
| 22 | cyclopentyl | —H | 2-chloro-6-fluorophenyl | —Cl | —CN | | 4.13 | |
| 23 | i-propyl | —H | 2-chloro-6-fluorophenyl | —Cl | —CN | | 3.65 | |
| 24 | 2-methoxyethyl | —H | 2-chloro-6-fluorophenyl | —Cl | —CN | | 3.22 | |
| 25 | cyclopropyl | —H | 2-chloro-6-fluorophenyl | —Cl | —CN | | 3.37 | |
| 26 | —CH₂—CH₂—S—CH₂—CH₂— | * | 2-chloro-6-fluorophenyl | —Cl | —CN | | 3.9 | |
| 27 | —CH₂—CH₂—CH(CF₃)—CH₂—CH₂— | * | 2-chloro-6-fluorophenyl | —Cl | —CN | | 4.37 | |
| 28 | —CH₂—CH₂—CH(CH₃)—CH₂—CH₂— | * | 2-chloro-6-fluorophenyl | —Cl | —CN | | 4.77 | |
| 29 | cyclopropylmethyl | —H | 2-chloro-6-fluorophenyl | —Cl | —CN | | 3.74 | |
| 30 | 2-butyl | —H | 2-chloro-6-fluorophenyl | —Cl | —CN | | 3.94 | |
| 31 | —CH₂—CH₂—CH═CH—CH₂— | * | 2-chloro-6-fluorophenyl | —Cl | —CN | | 4.08 | |
| 32 | —CH₂—CH₂—CH₂—CH(CH₃)—CH₂— | * | 2-chloro-6-fluorophenyl | —Cl | —CN | | 4.77 | |
| 33 | —CH₂—CH₂—CH═C(CH₃)—CH₂— | * | 2-chloro-6-fluorophenyl | —Cl | —CN | | 4.51 | |
| 34 | —CH₂—CH₂—CHF—CH₂—CH₂— | * | 2-chloro-6-fluorophenyl | —Cl | —CN | | 3.82 | |
| 35 | allyl | —C₂H₅ | 2-chloro-6-fluorophenyl | —Cl | —CN | | 4.32 | |
| 36 | (2-furyl)methyl | —C₂H₅ | 2-chloro-6-fluorophenyl | —Cl | —CN | | 4.32 | |
| 37 | (2-tetrahydrofuryl)methyl | —C₂H₅ | 2-chloro-6-fluorophenyl | —Cl | —CN | | 4.08 | |
| 38 | 2-methoxyethyl | —C₂H₅ | 2-chloro-6-fluorophenyl | —Cl | —CN | | 3.86 | |
| 39 | —CH₂—COOC₂H₅ | —C₂H₅ | 2-chloro-6-fluorophenyl | —Cl | —CN | | 3.86 | |
| 40 | propargyl | —CH₃ | 2-chloro-6-fluorophenyl | —Cl | —CN | | 3.53 | |
| 41 | —CH₂—COOC₂H₅ | —CH₃ | 2-chloro-6-fluorophenyl | —Cl | —CN | | 3.57 | |
| 42 | 1,3-dioxolan-2-ylmethyl | —CH₃ | 2-chloro-6-fiuorophenyl | —Cl | —CN | | 3.49 | |
| 43 | allyl | —CH₃ | 2-chloro-6-fluorophenyl | —Cl | —CN | | 4.03 | |
| 44 | (2-furyl)methyl | —CH₃ | 2-chloro-6-fluorophenyl | —Cl | —CN | | 3.99 | |
| 45 | —CH₂—C(CH₃)═CH₂ | —CH₃ | 2-chloro-6-fluorophenyl | —Cl | —CN | | 4.37 | |
| 46 | i-butyl | —CH₃ | 2-chloro-6-fluorophenyl | —Cl | —CN | | 4.51 | |
| 47 | (2-tetrahydrofuryl)methyl | n-propyl | 2-chloro-6-fluorophenyl | —Cl | —CN | | 4.51 | |
| 48 | i-butyl | —H | 2,4,6-trifluorophenyl | —Cl | —CN | | 3.85 | |
| 49 | —CH₂—C(CH₃)₃ | —H | 2,4,6-trifluorophenyl | —Cl | —CN | | 4.26 | |
| 50 | 2-butyl | —H | 2,4,6-trifluorophenyl | —Cl | —CN | | 3.89 | |
| 51 | cyclopentyl | —H | 2,4,6-trifluorophenyl | —Cl | —CN | | 4.01 | |
| 52 | i-propyl | —H | 2,4,6-trifluorophenyl | —Cl | —CN | | 3.54 | |
| 53 | cyclopropyl | —H | 2,4,6-trifluorophenvl | —Cl | —CN | | 3.25 | |
| 54 | cyclopropylmethyl | —H | 2,4,6-trifluorophenyl | —Cl | —CN | | 3.63 | |
| 55 | —CH₂—C(CH₃)═CH₂ | —H | 2,4,6-trifluorophenyl | —Cl | —CN | | 3.54 | |
| 56 | 1,3-dioxolan-2-ylmethyl | —CH₃ | 2,4,6-trifluorophenyl | —Cl | —CN | | 3.33 | |
| 57 | 2-methoxyethyl | —C₂H₅ | 2,4,6-trifluorophenyl | —Cl | —CN | | 3.74 | |
| 58 | —CH₂—CH₂—CH₂—CH₂—CH(CH₃)— | * | 2,4,6-trifluorophenyl | —Cl | —CN | | 4.5 | |
| 59 | 2-butyl | —H | 2-fluorophenyl | —Cl | —CN | | 3.7 | |
| 60 | —CH₂—CH₂—CF₃ | —H | 2-fluorophenyl | —Cl | —CN | | 3.34 | |
| 61 | n-propyl | —H | 2-fluorophenyl | —Cl | —CN | | 3.38 | |
| 62 | i-propyl | —H | 2-fluorophenyl | —Cl | —CN | | 3.36 | |
| 63 | cyclohexyl | —H | 2-fluorophenyl | —Cl | —CN | | 4.2 | |
| 64 | 1-cyclohexylethyl | —H | 2-fluorophenyl | —Cl | —CN | | 4.91 | |
| 65 | 2-methoxyethyl | —H | 2-fluorophenyl | —Cl | —CN | | 2.89 | |
| 66 | cyclopropyl | —H | 2-fluorophenyl | —Cl | —CN | | 3.11 | |
| 67 | —CH₂—CF₃ | —H | 2-fluorophenyl | —Cl | —CN | | 3.15 | |
| 68 | —CH₂—C(CH₃)═CH₂ | —H | 2-fluorophenyl | —Cl | —CN | | 3.39 | |
| 69 | 3-trifluoromethylcyclohexyl | —H | 2-fluorophenyl | —Cl | —CN | | 4.15 | |
| 70 | 2-trifluoromethylcyclohexyl | —H | 2-fluorophenyl | —Cl | —CN | | 4.26 | |
| 71 | 3,5- | —H | 2-fluorophenyl | —Cl | —CN | | 4.26 | |
| | bistrifluoromethylcyclohexyl | | | | | | | |
| 72 | —C₂H₅ | —C₂H₅ | 2-fluorophenyl | —Cl | —CN | | 3.8 | |
| 73 | —CH₂—CH₂—O—CH₂—CH₂— | * | 2-fluorophenyl | —Cl | —CN | | 2.88 | |
| 74 | 2,2,2-trifluoro-1- | —H | 2-fluorophenyl | —Cl | —CN | S | 3.49 | |
| | methylethyl | | | | | | | |
| 75 | —CH(CH₃)—CH₂—CH(CH₃)₂ | —H | 2-fluorophenyl | —Cl | —CN | | | |
| 76 | i-butyl | —H | 2-chlorophenyl | —Cl | —CN | | 4 | |
| 77 | —CH₂—C(CH₃)₃ | —H | 2-chlorophenyl | —Cl | —CN | | 4.47 | |
| 78 | 2-butyl | —H | 2-chlorophenyl | —Cl | —CN | | 3.98 | |
| 79 | cyclopentyl | —H | 2-chlorophenyl | —Cl | —CN | | 4.19 | |
| 80 | i-propyl | —H | 2-chlorophenyl | —Cl | —CN | | 3.64 | |
| 81 | cyclopropyl | —H | 2-chlorophenyl | —Cl | —CN | | 3.38 | |
| 82 | cyclopropylmethyl | —H | 2-chlorophenyl | —Cl | —CN | | 3.74 | |
| 83 | —CH₂—C(CH₃)═CH₂ | —H | 2-chlorophenyl | —Cl | —CN | | 3.68 | |
| 84 | —CH(CH₃)—CH₂—CH(CH₃)₂ | —H | 2-chlorophenyl | —Cl | —CN | | 4.7 | |
| 85 | 1,3-dioxolan-2-ylmethyl | —CH₃ | 2-chlorophenyl | —Cl | —CN | | 3.42 | |
| 86 | allyl | —CH₃ | 2-chlorophenyl | —Cl | —CN | | 4.03 | |
| 87 | 2-methoxyethyl | —CH₃ | 2-chlorophenyl | —Cl | —CN | | 3.5 | |
| 88 | —CH₂—C(CH₃)═CH₂ | —CH₃ | 2-chlorophenyl | —Cl | —CN | | 4.39 | |
| 89 | —CH₂—C(CH₃)═CH₂ | —C₂H₅ | 2-chlorophenyl | —Cl | —CN | | 3.68 | |
| 90 | allyl | —C₂H₅ | 2-chlorophenyl | —Cl | —CN | | 4.32 | |
| 91 | —CH₂—CH₂—CH₂—CH(CH₃)— | * | 2-chlorophenyl | —Cl | —CN | | 4.18 | |
| 92 | —CH₂—CH₂—CH₂—CH₂— | * | 2-chlorophenyl | —Cl | —CN | | 3.82 | |
| 93 | —CH₂—CH₂—CH═CH—CH₂— | * | 2-chlorophenyl | —Cl | —CN | | 4.1 | |
| 94 | —CH₂—CH₂—CH₂—CH₂—CH(CH₃)— | * | 2-chlorophenyl | —Cl | —CN | | 4.69 | |
| 95 | —CH₂—CH₂—CH(CH₃)—CH₂—CH₂— | * | 2-chlorophenyl | —Cl | —CN | | 4.78 | |
| 96 | —CH₂—CH₂—CH═C(CH₃)—CH₂— | * | 2-chlorophenyl | —Cl | —CN | | 4.52 | |
| 97 | —CH₂—CH₂—CH(CF₃)—CH₂—CH₂— | * | 2-chlorophenyl | —Cl | —CN | | 4.35 | |
| 98 | —CH₂—CH₂—CH₂—CH₂—CH₂— | * | 2-chlorophenyl | —Cl | —CN | | 4.36 | |
| 99 | —CH₂—CH₂—O—CH₂—CH₂— | * | 2-chlorophenyl | —Cl | —CN | | 3.17 | |
| 100 | —CH₂—CH₂—S—CH₂—CH₂— | * | 2-chlorophenyl | —Cl | —CN | | 3.88 | |
| 101 | —CH₂—CH₂—N(CH₃)₂ | —C₂H₅ | 2-chloro-6-fluorophenyl | —Cl | —CN | | 1.9 | |
| 102 | —CH(CF₃)—CH₂—CH₂—CH₂— | * | 2-chloro-6-fluorophenyl | —Cl | —CN | | 4.18 | |
| 103 | 2,2,2-trifluoro-1-methylethyl | —H | 2-chlorophenyl | —Cl | —CN | S | 3.79 | |
| 104 | —CH₂—CH₂—CH₂—CH₂—CH₂— | * | 2-fluorophenyl | —Cl | —CN | | 4.05 | |
| 105 | —CH₂—C(CH₃)═CH₂ | —C₂H₅ | 2,4-difluorophenyl | —Cl | —CN | | 4.47 | 95-98 |
| 106 | —CH₂—C(CH₃)═CH₂ | —C₂H₅ | 2,4,6-trifluorophenyl | —Cl | —Cl | | 5.55 | |
| 107 | allyl | —CH₃ | 2,4,6-trifluorophenyl | —Cl | —CN | | 3.87 | |
| 108 | i-butyl | —CH₃ | 2,4,6-trifluorophenyl | —Cl | —CN | | 4.37 | |
| 109 | 2-methoxyethyl | —CH₃ | 2,4,6-trifluorophenvl | —Cl | —CN | | 3.44 | |
| 110 | —CH₂—C(CH₃)═CH₂ | —CH₃ | 2,4,6-trifluorophenyl | —Cl | —CN | | 4.24 | |
| 111 | allyl | —C₂H₅ | 2,4,6-trifluorophenyl | —Cl | —CN | | 4.23 | |
| 112 | —CH₂—CH₂—CH₂—CH(CH₃)— | * | 2,4,6-trifluorophenyl | —Cl | —CN | | 4.09 | |
| 113 | —CH(CF₃)—CH₂—CH₂—CH₂— | * | 2,4,6-trifluorophenyl | —Cl | —CN | | 4.12 | |
| 114 | —CH₂—CH₂—CH₂—CH₂— | * | 2,4,6-trifluorophenyl | —Cl | —CN | | 3.71 | |
| 115 | —CH₂—CH₂—CH═CH—CH₂— | * | 2,4,6-trifluorophenyl | —Cl | —CN | | 3.96 | |
| 116 | —CH₂—CH₂—CH₂—CH(CH₃)—CH₂— | * | 2,4,6-trifluorophenyl | —Cl | —CN | | 4.6 | |
| 117 | —CH₂—CH₂—CH(CH₃)—CH₂—CH₂— | * | 2,4,6-trifluorophenyl | —Cl | —CN | | 4.6 | |
| 118 | —CH₂—CH₂—CH═C(CH₃)—CH₂— | * | 2,4,6-trifluorophenyl | —Cl | —CN | | 4.34 | |
| 119 | —CH₂—CH₂—CHF—CH₂—CH₂— | * | 2,4,6-trifluorophenyl | —Cl | —CN | | 3.72 | |
| 120 | —CH₂—CH₂—CH(CF₃)—CH₂—CH₂— | * | 2,4,6-trifluorophenyl | —Cl | —CN | | 4.26 | |
| 121 | —CH₂—CH₂—CH₂—CH₂—CH₂— | * | 2,4,6-trifluorophenyl | —Cl | —CN | | 4.23 | |
| 122 | —CH₂—CH₂—O—CH₂—CH₂— | * | 2,4,6-trifluorophenyl | —Cl | —CN | | 3.16 | |
| 123 | —CH₂—CH₂—S—CH₂—CH₂— | * | 2,4,6-trifluorophenyl | —Cl | —CN | | 3.79 | |
| 124 | —CH₂—CF₃ | —H | 2,4,6-trifluorophenyl | —Cl | —CN | | 3.37 | |
| 125 | —C₂H₅ | —C₂H₅ | 2,4-difluorophenyl | —Cl | —CN | | | |
| 126 | 2,2,2-trifluoro-1- | —H | 2,4-difluorophenyl | —Cl | —CN | S | 3.65 | 123-25 |
| | methylethyl | | | | | | | |
| 127 | —C₂H₅ | —H | 2-fluorophenyl | —Cl | —CN | | 3.06 | |
| 128 | —CH₂—CN | —H | 2-fluorophenyl | —Cl | —CN | | 2.45 | |
| 129 | —C(CH₃)₂—CF₃ | —H | 2-fluorophenyl | —Cl | —CN | | 4.01 | |
| 130 | 4-trifluoromethylcyclohexyl | —H | 2-fluorophenyl | —Cl | —CN | | 4.2 | |
| 131 | —CH₃ | —CH₃ | 2-fluorophenyl | —Cl | —CN | | 3.12 | |
| 132 | —CH₂—CH₂—CH₂—CH₂— | * | 2-fluorophenyl | —Cl | —CN | | 3.56 | |
| 133 | —CH₂—CH₂—CH(CF₃)—CH₂—CH₂— | * | 2-fluorophenyl | —CI | —CN | | 4.13 | |
| 134 | —CH₂—CH(CH₃)—O—CH(CH₃)— | * | 2-fluorophenyl | —Cl | —CN | | 3.67 | |
| | CH₂— | | | | | | | |
| 135 | —CH₂—CH₂—S—CH₂—CH₂— | * | 2-fluorophenyl | —Cl | —CN | | 3.63 | |
| 136 | 1-cyclopropylethyl | —H | 2,4,6-trifluorophenyl | —Cl | —Cl | | 4.66 | |
| 137 | —CH₂—CF₃ | —H | 2-chlorophenyl | —Cl | —CN | | 3.43 | |
| 138 | i-butyl | —CH₃ | 2-chlorophenyl | —Cl | —CN | | 4.51 | |
| 139 | 2-methoxyethyl | n-propyl | 2-chlorophenyl | —Cl | —CN | | 4.23 | |
| 140 | 2-methoxyethyl | —C₂H₅ | 2-chlorophenyl | —Cl | —CN | | 4.28 | |
| 141 | —CH(CF₃)—CH₂—CH₂—CH₂— | * | 2-chlorophenyl | —Cl | —CN | | 4.19 | |
| 142 | —CH₂—CH₂—CH₂—CH(CH₃)—CH₂— | * | 2-chlorophenyl | —Cl | —CN | | 4.82 | |
| 143 | —CH₂—CH₂—CHF—CH₂—CH₂— | * | 2-chlorophenyl | —Cl | —CN | | 3.81 | |
| 144 | i-propyl | —H | 2-chloro-4-fluorophenyl | —Cl | —CN | | 3.78 | |
| 145 | 2,2,2-trifluoro-1-methylethyl | —H | 2-chloro-4-fluorophenyl | —Cl | —CN | AS + BR | 3.87 | |
| 146 | 2,2,2-trifluoro-1- | —H | 2-chloro-4-fluorophenyl | —Cl | —CN | AS + BR + | 3.92 | |
| | methylethyl | | | | | BS + AR | | |
| 147 | 2,2,2-trifluoro-1-methylethyl | —H | 2-chloro-4-fluorophenyl | —Cl | —CN | AS + BR | 3.91 | |
| 148 | i-butyl | —H | 2,4-difluorophenyl | —Cl | —CN | | 3.87 | |
| 149 | n-butyl | —H | 2,4-difluorophenyl | —Cl | —CN | | 3.86 | |
| 150 | —CH₂—C(CH₃)₃ | —H | 2,4-difluorophenyl | —Cl | —CN | | 4.23 | |
| 151 | 2-butyl | —H | 2,4-difluorophenyl | —Cl | —CN | | 3.82 | |
| 152 | —CH₂—CH₂—CF₃ | —H | 2,4-difluorophenyl | —Cl | —CN | | 3.47 | |
| 153 | n-propyl | —H | 2,4-difluorophenyl | —Cl | —CN | | 3.5 | |
| 154 | cyclopentyl | —H | 2,4-difluorophenyl | —Cl | —CN | | 3.98 | |
| 155 | i-propyl | —H | 2,4-difluorophenyl | —Cl | —CN | | 3.5 | |
| 156 | cyclohexyl | —H | 2,4-difluorophenyl | —Cl | —CN | | 4.26 | |
| 157 | 1-cyclohexylethyl | —H | 2,4-difluorophenyl | —Cl | —CN | | 4.96 | |
| 158 | 2-methoxyethyl | —H | 2,4-difluorophenyl | —Cl | —CN | | 3.06 | |
| 159 | cyclopropyl | —H | 2,4-difluorophenyl | —Cl | —CN | | 3.23 | |
| 160 | cyclopropylmethyl | —H | 2,4-difluorophenyl | —Cl | —CN | | 4.35 | |
| 161 | —CH₂—C(CH₃)═CH₂ | —H | 2,4-difluorophenyl | —Cl | —CN | | 3.51 | |
| 162 | 3-trifluoromethylcyclohexyl | —H | 2,4-difluorophenyl | —Cl | —CN | | 4.2 | |
| 163 | 2-trifluoromethylcyclohexyl | —H | 2,4-difluorophenyl | —Cl | —CN | | 4.23 | |
| 164 | 4-trifluoromethylcyclohexyl | —H | 2,4-difluorophenyl | —Cl | —CN | | 4.21 | |
| 165 | —CH(CH₃)—CH₂—CH(CH₃)₂ | —H | 2,4-difluorophenyl | —Cl | —CN | | 4.47 | |
| 166 | —CH₂—CH₂—N(CH₃)₂ | —CH₃ | 2,4-difluorophenyl | —Cl | —CN | | 1.72 | |
| 167 | propargyl | —CH₃ | 2,4-difluorophenyl | —Cl | —CN | | 3.35 | |
| 168 | 1,3-dioxolan-2-ylmethyl | —CH₃ | 2,4-difluorophenyl | —Cl | —CN | | 3.3 | |
| 169 | —CH₂—CH(OCH₃)₂ | —CH₃ | 2,4-difluorophenyl | —Cl | —CN | | 3.46 | |
| 170 | —CH₂—C(CH₃)═CH₂ | —CH₃ | 2,4-difluorophenyl | —Cl | —CN | | 4.16 | |
| 171 | n-butyl | —CH₃ | 2,4-difluorophenyl | —Cl | —CN | | 4.36 | |
| 172 | i-butyl | —H | 2,6-difluorophenyl | —Cl | —CN | | 3.73 | |
| 173 | —CH₂—C(CH₃)₃ | —H | 2,6-difluorophenyl | —Cl | —CN | | 4.15 | |
| 174 | 2-butyl | —H | 2,6-difluorophenyl | —Cl | —CN | | 3.71 | |
| 175 | —CH₂—CN | —H | 2,6-difluorophenyl | —Cl | —CN | | 2.49 | |
| 176 | cyclopentyl | —H | 2,6-difluorophenyl | —Cl | —CN | | 3.89 | |
| 177 | 1-propyl | —H | 2,6-difluorophenyl | —Cl | —CN | | 3.39 | |
| 178 | 2-methoxyethyl | —H | 2,6-difluorophenyl | —Cl | —CN | | 2.96 | |
| 179 | cyclopropyl | —H | 2,6-difluorophenyl | —Cl | —CN | | 3.13 | |
| 180 | —CH₂—CF₃ | —H | 2,6-difluorophenyl | —Cl | —CN | | 3.07 | |
| 181 | cyclopropylmethyl | —H | 2,6-difluorophenyl | —Cl | —CN | | 3.5 | |
| 182 | —CH₂—C(CH₃)═CH₂ | —H | 2,6-difluorophenyl | —Cl | —CN | | 3.4 | |
| 183 | —CH(CH₃)—CH₂—CH(CH₃)₂ | —H | 2,6-difluorophenyl | —Cl | —CN | | 4.39 | |
| 184 | propargyl | —CH₃ | 2,6-difluorophenyl | —Cl | —CN | | 3.27 | |
| 185 | —CH₂—COOC₂H₅ | —CH₃ | 2,6-difluorophenyl | —Cl | —CN | | 3.31 | |
| 186 | 1,3-dioxolan-2-ylmethyl | —CH₃ | 2,6-difluorophenyl | —Cl | —CN | | 3.21 | |
| 187 | allyl | —CH₃ | 2,6-difluorophenyl | —Cl | —CN | | 3.77 | |
| 188 | i-butyl | —CH₃ | 2,6-difluorophenyl | —Cl | —CN | | 4.23 | |
| 189 | 2-methoxyethyl | —CH₃ | 2,6-difluorophenyl | —Cl | —CN | | 3.27 | |
| 190 | —CH₂—C(CH₃)═CH₂ | —CH₃ | 2,6-difluorophenyl | —Cl | —CN | | 4.1 | |
| 191 | allyl | —C₂H₅ | 2,6-difluorophenyl | —Cl | —CN | | 4.07 | |
| 192 | (2-furyl)methyl | —C₂H₅ | 2,6-difluorophenyl | —Cl | —CN | | 4.04 | |
| 193 | (2-tetrahydrofuryl)methyl | —C₂H₅ | 2,6-difluorophenyl | —Cl | —CN | | 3.84 | |
| 194 | 2-methoxyethyl | —C₂H₅ | 2,6-difluorophenyl | —Cl | —CN | | 3.59 | |
| 195 | —CH₂—COOC₂H₅ | —C₂H₅ | 2,6-difluorophenyl | —Cl | —CN | | 3.61 | |
| 196 | n-butyl | —C₂H₅ | 2,6-difluorophenyl | —Cl | —CN | | 4.64 | |
| 197 | —C₂H₅ | —C₂H₅ | 2,6-difluorophenyl | —Cl | —CN | | 3.88 | |
| 198 | cyclopropylmethyl | n-propyl | 2,6-difluorophenyl | —Cl | —CN | | 4.65 | |
| 199 | (2-tetrahydrofuryl)methyl | n-propyl | 2,6-difluorophenyl | —Cl | —CN | | 4.24 | |
| 200 | 2-methoxyethyl | n-propyl | 2,6-difluorophenyl | —Cl | —CN | | 3.96 | |
| 201 | —CH₂—CH(OH)—CH₂CH₂— | * | 2,6-difluorophenyl | —Cl | —CN | | 2.47 | |
| 202 | —CH₂—CH₂—CH₂—CH(CH₃)— | * | 2,6-difluorophenyl | —Cl | —CN | | 3.92 | |
| 203 | —CH₂—CH₂—CH₂—CH₂— | * | 2,6-difluorophenyl | —Cl | —CN | | 3.55 | |
| 204 | —CH₂—CH₂—CH₂—CH₂—CH(CH₃)— | * | 2,6-difluorophenyl | —Cl | —CN | | 4.4 | |
| 205 | —CH₂—CH₂—CH₂—CH(CH₃)—CH₂— | * | 2,6-difluorophenyl | —Cl | —CN | | 4.46 | |
| 206 | —CH₂—CH₂—CH(CH₃)—CH₂—CH₂— | * | 2,6-difluorophenyl | —Cl | —CN | | 4.46 | |
| 207 | —CH₂—CH₂—CH═C(CH₃)—CH₂— | * | 2,6-difluorophenyl | —Cl | —CN | | 4.2 | |
| 208 | —CH₂—CH₂—CH(CF₃)—CH₂—CH₂— | * | 2,6-difluorophenyl | —Cl | —CN | | 4.13 | |
| 209 | —CH₂—CH₂—CH₂—CH₂—CH₂— | * | 2,6-difluorophenyl | —Cl | —CN | | 4.07 | |
| 210 | —CH₂—CH₂—S—CH₂—CH₂— | * | 2,6-difluorophenyl | —Cl | —CN | | 3.65 | |
| 211 | 2-fluorocyclopropyl | —H | 2,4,6-trifluorophenyl | —Cl | —CN | | 3.06 | |
| 212 | i-butyl | —H | 2,4,6-trifluorophenyl | —Cl | —Cl | | 4.7 | |
| 213 | allyl | —C₂H₅ | 2,4,6-trifluorophenyl | —Cl | —Cl | | 5.14 | |
| 214 | 2-methoxyethyl | —C₂H₅ | 2,4,6-trifluorophenyl | —Cl | —Cl | | 4.61 | |
| 215 | —CH₂—CH₂—CH₂—CH(CH₃)— | * | 2,4,6-trifluorophenyl | —Cl | —Cl | | 4.99 | |
| 216 | —CH₂—CH₂—CH₂—CH₂— | * | 2,4,6-trifluorophenyl | —Cl | —Cl | | 4.56 | |
| 217 | —CH₂—CH₂—CH(CH₃)—CH₂—CH₂— | * | 2,4,6-trifluorophenyl | —Cl | —Cl | | 5.59 | |
| 218 | —CH₂—CH₂—CH═CH—CH₂— | * | 2,4,6-trifluorophenyl | —Cl | —Cl | | 4.84 | |
| 219 | —CH₂—CH₂—CH₂—CH(CH₃)—CH₂— | * | 2,4,6-trifluorophenyl | —Cl | —Cl | | 5.59 | |
| 220 | —CH₂—CH₂—CH₂—CH₂—CH₂— | * | 2,4,6-trifluorophenyl | —Cl | —Cl | | 5.14 | |
| 221 | —CH₂—CH₂—O—CH₂—CH₂— | * | 2,4,6-trifluorophenyl | —Cl | —Cl | | 3.94 | |
| 222 | —CH₂—C(CH₃)₃ | —H | 2,4,6-trifluorophenyl | —Cl | —Cl | | 5.19 | |
| 223 | cyclopropylmethyl | —H | 2,4,6-trifluorophenyl | —Cl | —Cl | | 4.41 | |
| 224 | —CH₂—CF₃ | —H | 2,4,6-trifluorophenyl | —Cl | —Cl | | 4.08 | |
| 225 | —CH₂—C(CH₃)═CH₂ | —H | 2,4,6-trifluorophenyl | —Cl | —Cl | | 4.32 | |
| 226 | allyl | —CH₃ | 2,4,6-trifluorophenyl | —Cl | —Cl | | 4.8 | |
| 227 | i-butyl | —CH₃ | 2,4,6-trifluorophenyl | —Cl | —Cl | | 5.31 | |
| 228 | 2-methoxyethyl | —CH₃ | 2,4,6-trifluorophenyl | —Cl | —Cl | | 4.23 | |
| 229 | —CH₂—C(CH₃)═CH₂ | —CH₃ | 2,4,6-trifluorophenyl | —Cl | —Cl | | 5.17 | |
| 230 | —CH₂—CH₂—CF₂—CH₂—CH₂— | * | 2,4,6-trifluorophenyl | —Cl | —CN | | 3.81 | |
| 231 | —CH₂—CH₂—CF₂—CH₂—CH₂— | * | 2,4,6-trifluorophenyl | —Cl | —Cl | | 4.61 | |
| 232 | (2,2- | —CH₃ | 2,4,6-trifluorophenyl | —Cl | —CN | | 4.32 | |
| | dichlorocyclopropyl)methyl | | | | | | | |
| 233 | (2,2- | —CH₃ | 2,4,6-trifluorophenyl | —Cl | —Cl | | 5.16 | |
| | dichlorocyclopropyl)methyl | | | | | | | |
| 234 | 2-fluorocyclopropyl | —H | 2,4,6-trifluorophenyl | —Cl | —Cl | | 3.72 | |
| 235 | —C₂H₅ | —H | 2,4-difluorophenyl | —Cl | —CN | | 3.2 | |
| 236 | —CH₂—CF₃ | —H | 2,4-difluorophenyl | —Cl | —CN | | 3.34 | |
| 237 | 3,5- | —H | 2,4-difluorophenyl | —Cl | —CN | | 4.41 | |
| | bistrifluoromethylcyclohexyl | | | | | | | |
| 238 | —CH₂—COOC₂H₅ | —CH₃ | 2,4-difluorophenyl | —Cl | —CN | | 3.49 | |
| 239 | allyl | —CH₃ | 2,4-difluorophenyl | —Cl | —CN | | 3.87 | |
| 240 | —CH₂—CH₂—CN | —CH₃ | 2,4-difluorophenyl | —Cl | —CN | | 2.98 | |
| 241 | —CH₂—CN | —CH₃ | 2,4-difluorophenyl | —Cl | —CN | | 2.95 | |
| 242 | —CH₂—COOCH₃ | —CH₃ | 2,4-difluorophenyl | —Cl | —CN | | 3.17 | |
| 243 | (2-furyl)methyl | —CH₃ | 2,4-difluorophenyl | —Cl | 13 CN | | 3.87 | |
| 244 | i-butyl | —CH₃ | 2,4-difluorophenyl | —Cl | —CN | | 4.33 | |
| 245 | —CH₂—CH₂—O—CH═CH₂ | —CH₃ | 2,4-difluorophenyl | —Cl | —CN | | 2.6 | |
| 246 | 2-methoxyethyl | —CH₃ | 2,4-difluorophenyl | —Cl | —CN | | 3.41 | |
| 247 | —CH₃ | —CH₃ | 2,4-difluorophenyl | —Cl | —CN | | 3.25 | |
| 248 | 1,2-dimethylpropyl | —H | 2,4,6-trifluorophenyl | —Cl | —CN | | 4.17 | |
| 249 | 1,2-dimethylpropyl | —H | 2,4,6-trifluorophenyl | —Cl | —Cl | | 5.02 | |
| 250 | 1,2-dimethylpropyl | —H | 2,4,6-trifluorophenyl | —Cl | —Cl | | 5.02 | |
| 251 | 1,2-dimethylpropyl | —H | 2,4,6-trifluorophenyl | —Cl | —Cl | | 5.02 | |
| 252 | 1,2-dimethylpropyl | —H | 2,4,6-trifluorophenyl | —Cl | —CN | | 4.16 | |
| 253 | 1,2-dimethyipropyl | —H | 2,4,6-trifluorophenyl | —Cl | —CN | | 4.16 | |
| 254 | —O—CH₂—CH₂—CH₂—CH₂— | * | 2,4-difluorophenyl | —Cl | —CN | | 3.71 | |
| 255 | —O—CH₂—CH₂—CH₂—CH₂— | * | 2-chloro-4-fluorophenyl | —Cl | —CN | | 4.02 | |
| 256 | —O—CH₂—CH₂—CH₂—CH₂— | * | 2-chloro-6-fluorophenyl | —Cl | —CN | | 3.85 | |
| 257 | 1,2-dimethylpropyl | —H | 2-chloro-4-fluorophenyl | —Cl | —CN | AS + BR | 4.43 | |
| 258 | 1,2-dimethylpropyl | —H | 2-chloro-4-fluorophenyl | —Cl | —CN | AR + BS | 4.48 | |
| 259 | i-butyl | —H | 2-chloro-4-fluorophenyl | —Cl | —CN | | 4.18 | |
| 260 | —CH₂—C(CH₃)₃ | —H | 2-chloro-4-fluorophenyl | —Cl | —CN | | 4.61 | |
| 261 | 2-butyl | —H | 2-chloro-4-fluorophenyl | —Cl | —CN | | 4.18 | |
| 262 | cyclopentyl | —H | 2-chloro-4-fluorophenyl | —Cl | —CN | | 4.32 | |
| 263 | 2-methoxyethyl | —H | 2-chloro-4-fluorophenyl | —Cl | —CN | | 3.33 | |
| 264 | cyclopropylmethyl | —H | 2-chloro-4-fluorophenyl | —Cl | —CN | | 3.9 | |
| 265 | —CH₂—C(CH₃)═CH₂ | —H | 2-chloro-4-fluorophenyl | —Cl | —CN | | 3.85 | |
| 266 | i-butyl | —CH₃ | 2-chloro-4-fluorophenyl | —Cl | —CN | | 4.67 | |
| 267 | 2-methoxyethyl | —CH₃ | 2-chloro-4-fluorophenyl | —Cl | —CN | | 3.72 | |
| 268 | —CH₂—C(CH₃)═CH₂ | —CH₃ | 2-chloro-4-fluorophenyl | —Cl | —CN | | 4.5 | |
| 269 | —CH₂—C(CH₃)═CH₂ | —C₂H₅ | 2-chloro-4-fluorophenyl | —Cl | —CN | | 4.87 | |
| 270 | 2-methoxyethyl | —C₂H₅ | 2-chloro-4-fluorophenyl | —Cl | —CN | | 3.99 | |
| 271 | —CH₂—CH₂—CH₂—CH(CH₃)— | * | 2-chloro-4-fluorophenyl | —Cl | —CN | | 4.32 | |
| 272 | —CH₂—CH₂—CH₂—CH₂— | * | 2-chloro-4-fluorophenyl | —Cl | —CN | | 3.99 | |
| 273 | —CH₂—CH₂—CH(CH₃)—CH₂—CH₂— | * | 2-chloro-4-fluorophenyl | —Cl | —CN | | 4.92 | |
| 274 | —CH₂—CH₂—CH₂—CH₂—CH₂— | * | 2-chloro-4-fluorophenyl | —Cl | —CN | | 4.51 | |
| 275 | —CH₂—CH₂—O—CH₂—CH₂— | * | 2-chloro-4-fluorophenyl | —Cl | —CN | | 3.33 | |
| 276 | —CH₂—CF₃ | —H | 2-chloro-4-fluorophenyl | —Cl | —CN | | | |
| 277 | —CH(CF₃)—CH₂—CH₂—CH₂— | * | 2-chloro-4-fluorophenyl | —Cl | —CN | | | |
| 278 | 1,2-dimethylpropyl | —H | 2-chloro-6-fluorophenyl | —H | —Cl | | 4.43 | |
| 279 | —CH₂—C(CH₃)═CH₂ | —C₂H₅ | 2-chloro-4-fluorophenyl | —H | —Cl | | 5.14 | |
| 280 | —NH—CH₂—CH₂—CH₂—CH₂— | * | 2-chloro-4-fluorophenyl | —H | —Cl | | 3.57 | |
| 281 | —NH—CH₂—CH₂—CH₂—CH₂— | * | 2-chloro-6-fluorophenyl | —H | —Cl | | 3.6 | |
| 282 | —CH₂—CH₂—CH(COCH₃)—CH₂— | * | 2,4-difluorophenyl | —Cl | —CN | | 3.31 | |
| | CH₂— | | | | | | | |
| 283 | —CH₂—CH═C(C₂H₅)—CH₂—CH₂— | * | 2,4-difluorophenyl | —Cl | —CN | | 4.76 | |
| 284 | —CH₂—CH₂—CH═C(CH₃)—CH₂— | * | 2,4-difluorophenyl | —Cl | —CN | | 4.33 | |
| 285 | —CH₂—CH₂—CH(COOCH₃)—CH₂— | * | 2,4-difluorophenyl | —Cl | —CN | | 3.61 | |
| | CH₂— | | | | | | | |
| 286 | —CH₂—CH₂—CHBr—CH₂—CH₂— | * | 2,4-difluorophenyl | —Cl | —CN | | 4.21 | |
| 287 | —CH(COOCH₃)—CH₂—CH₂—CH₂— | * | 2,4-difluorophenyl | —Cl | —CN | | 3.85 | |
| | CH₂— | | | | | | | |
| 288 | —CH₂—CH₂—CHF—CH₂—CH₂— | * | 2,4-difluorophenyl | —Cl | —CN | | 3.66 | |
| 289 | 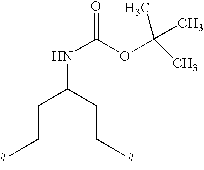 | * | 2,4-difluorophenyl | —Cl | —CN | | 4 | |
| 290 | —CH₂—CH₂—CH(CF₃)—CH₂—CH₂— | * | 2,4-difluorophenyl | —Cl | —CN | | 4.2 | |
| 291 | 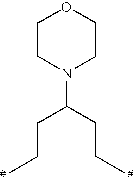 | * | 2,4-difluorophenyl | —Cl | —CN | | 1.74 | |
| 292 | —CH₂—CH₂—CH(NH—COCH₃)— | * | 2,4-difluorophenyl | —Cl | —CN | | 2.51 | |
| | CH₂—CH₂— | | | | | | | |
| 293 | —CH₂—CH₂—N(CH₃)—CH₂—CH₂— | * | 2,4-difluorophenyl | —Cl | —CN | | 1.47 | |
| 294 | —CH₂—CH(CH₃)—O—CH(CH₃)— | * | 2,4-difluorophenyl | —Cl | —CN | | 3.77 | |
| | CH₂— | | | | | | | |
| 295 | —CH₂—CH₂—CH₂—CH₂—CH₂— | * | 2,4-difluorophenyl | —Cl | —CN | | 4.18 | |
| 296 | —CH₂—CH₂—S—CH₂—CH₂— | * | 2,4-difluorophenyl | —Cl | —CN | | 3.73 | |
| 297 | 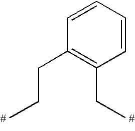 | * | 2,4-difluorophenyl | —Cl | —CN | | 4.38 | |
| 298 | 1,2-dimethylpropyl | —H | 2,6-difluorophenyl | —Cl | —CN | | 4.02 | |
| 299 | —CH₂—CHF₂ | —H | 2-chloro-6-fluorophenyl | —H | —Cl | | 3.09 | |
| 300 | 2-methoxyethyl | n-propyl | 2,4,6-trifluorophenyl | —Cl | —CN | | 4.1 | |
| 301 | 2,2,2-trifluoro-1-methylethyl | —H | 2,6-difluorophenyl | —Cl | —CN | R | 3.47 | |
| 302 | 1,2-dimethylpropyl | —H | 2-chloro-4-fluorophenyl | —Cl | —CN | BR | 4.44 | |
| 303 | 1,2-dimethylpropyl | —H | 2-chloro-4-fluorophenyl | —Cl | —CN | AR | 4.47 | |
| 304 | 1,2-dimethylpropyl | —H | 2-chloro-4-fluorophenyl | —Cl | —CN | AR + BR | 4.45 | |
| 305 | 1,2-dimethylpropyl | —H | 2-chloro-4-fluorophenyl | —Cl | —CN | AS | 4.46 | |
| 306 | 1,2-dimethylpropyl | —H | 2-chloro-4-fluorophenvl | —Cl | —CN | BS | 4.46 | |
| 307 | 1,2-dimethylpropyl | —H | 2-chloro-4-fluorophenyl | —Cl | —CN | AS + BS | 4.45 | |
| 308 | —CH₂—CH₂—N(CH₃)₂ | —C₂H₅ | 2,4-difluorophenyl | —Cl | —CN | | 1.83 | |
| 309 | allyl | —C₂H₅ | 2,4-difluorophenyl | —Cl | —CN | | 4.18 | |
| 310 | (2-furyl)methyl | —C₂H₅ | 2,4-difluorophenyl | —Cl | —CN | | 4.18 | |
| 311 | (2-tetrahydrofuryl)methyl | —C₂H₅ | 2,4-difluorophenyl | —Cl | —CN | | 4.02 | |
| 312 | —CH₂—CH₂—CN | —C₂H₅ | 2,4-difluorophenyl | —Cl | —CN | | 3.24 | |
| 313 | 2-methoxyethyl | —C₂H₅ | 2,4-difluorophenyl | —Cl | —CN | | 3.74 | |
| 314 | —CH₂—COOC₂H₅ | —C₂H₅ | 2,4-difluorophenyl | —Cl | —CN | | 3.81 | |
| 315 | 3-aminopropyl | n-propyl | 2,4-difluorophenyl | —Cl | —CN | | 1.75 | |
| 316 | (2-tetrahydrofuryl)methyl | n-propyl | 2,4-difluorophenyl | —Cl | —CN | | 4.45 | |
| 317 | 2-thienylmethyl | n-propyl | 2,4-difluorophenyl | —Cl | —CN | | 4.8 | |
| 318 | 2-methoxyethyl | n-propyl | 2,4-difluorophenyl | —Cl | —CN | | 4.13 | |
| 319 | —CH₂—CH₂—NH₂ | -i-propyl | 2,4-difluorophenyl | —Cl | —CN | | 1.72 | |
| 320 | —CH₂—COOC₂H₅ | cyclo- | 2,4-difluorophenyl | —Cl | —CN | | 3.99 | |
| | | propyl | | | | | | |
| 321 | —CH₂—CH(OH)—CH₂—CH₂— | * | 2,4-difluorophenyl | —Cl | —CN | | 2.57 | |
| 322 | —CH₂—CH₂—CH₂—CH(CH₃)— | * | 2,4-difluorophenyl | —Cl | —CN | | 4.05 | |
| 323 | —CH₂—CH₂—CH₂—CH₂— | * | 2,4-difluorophenyl | —Cl | —CN | | 3.7 | |
| 324 | —CH₂—CH₂—CH(OH)—CH₂—CH₂— | * | 2,4-difluorophenyl | —Cl | —CN | | 2.63 | |
| 325 | 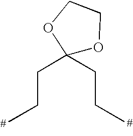 | * | 2,4-difluorophenyl | —Cl | —CN | | 3.51 | |
| 326 | —CH₂—CH(CH₃)—CH₂—CH(CH₃)— | * | 2,4-difluorophenyl | —Cl | —CN | | 4.98 | |
| | CH₂— | | | | | | | |
| 327 | —CH₂—CH₂—CH₂—CH₂—CH(CH₃)— | * | 2,4-difluorophenyl | —Cl | —CN | | 4.49 | |
| 328 | —CH₂—CH₂—CH₂—CH(CH₃)—CH₂— | * | 2,4-difluorophenyl | —Cl | —CN | | 4.59 | |
| 329 | —CH₂—CH₂—CH(CH₃)—CH₂—CH₂— | * | 2,4-difluorophenyl | —Cl | —CN | | 4.59 | |
| 330 | —CH₂—CH(OH)—CH₂—CH₂— | * | 2,4-difluorophenyl | —Cl | —CN | | 2.83 | |
| | CH₂— | | | | | | | |
| 331 | —CH₂—CH₂—C(CH₃)₂—CH₂—CH₂— | * | 2,4-difluorophenyl | —Cl | —CN | | 4.83 | |
| 332 | —CH₂—CH₂—S—CH₂—CH₂— | * | 2,4,6-trifluorophenyl | —Cl | —Cl | | 4.64 | |
| 333 | 1,2-dimethylpropyl | —H | 2-chlorophenyl | —Cl | —Cl | B | 5.17 | |
| 334 | 1,2-dimethylpropyl | —H | 2-chlorophenyl | —Cl | —Cl | A | 5.18 | |
| 335 | i-butyl | —CH₃ | 2-chlorophenyl | —Cl | —Cl | | 5.38 | |
| 336 | 1,2-dimethylpropyl | —H | 2-chloro-6-fluorophenyl | —Cl | —CN | | 4.25 | |
| 337 | 1,2-dimethylpropyl | —H | 2-chloro-6-fluorophenyl | —Cl | —CN | | 4.26 | |
| 338 | —CH₂—CH₂—O—CH₂—CH₂— | * | 2-chlorophenyl | —Cl | —Cl | | 3.86 | |
| 339 | —CH₂—CH₂—S—CH₂—CH₂ | * | 2-chlorophenyl | —Cl | —Cl | | 4.67 | |
| 340 | —CH₂—CH₂—CF₂—CH₂—CH₂ | * | 2-chlorophenyl | —Cl | —Cl | | 4.67 | |
| 341 | 2,2,2-trifluoro-1- | —H | 2-chlorophenyl | —Cl | —Cl | B | 4.48 | |
| | methylethyl | | | | | | | |
| 342 | 2,2,2-trifluoro-1- | —H | 2-chlorophenyl | —Cl | —CI | A | 4.52 | |
| | methylethyl | | | | | | | |
| 343 | —CH₂—CH₂—O—CH₂—CH₂— | * | 2-chloro-4-fluorophenyl | —Cl | —Cl | | 4.01 | |
| 344 | —CH₂—CH₂—S—CH₂—CH₂ | * | 2-chloro-4-fluorophenyl | —Cl | —Cl | | 4.79 | |
| 345 | —CH₂—CH₂—CF₂—CH₂—CH₂— | * | 2-chloro-4-fluorophenyl | —Cl | —Cl | | 4.79 | |
| 346 | cyclopropylmethyl | —H | 2-chloro-4-fluorophenyl | —Cl | —Cl | | 4.57 | |
| 347 | —CH₂—CF₃ | —H | 2-chloro-4-fluorophenyl | —Cl | —Cl | | 4.2 | |
| 348 | i-butyl | —CH₃ | 2-trifluoromethylphenyl | —Cl | —CN | | 4.48 | |
| 349 | —CH₂—C(CH₃)₃ | —H | 2-trifluoromethylphenyl | —Cl | —CN | | 4.43 | |
| 350 | —CH₂—CH₂—CH(CH₃)—CH₂—CH₂ | * | 2-trifluoromethylphenyl | —Cl | —CN | | 4.71 | |
| 351 | 2,2,2-trifluoro-1- | —H | 2-chloro-4-fluorophenyl | —Cl | —Cl | B | 4.63 | |
| | methylethyl | | | | | | | |
| 352 | 2,2,2-trifluoro-1- | —H | 2-chloro-4-fluorophenyl | —Cl | —Cl | A | 4.62 | |
| | methylethyl | | | | | | | |
| 353 | 1,2-dimethylpropyl | —H | 2-chloro-4-fluorophenyl | —Cl | —Cl | B | 5.26 | |
| 354 | 1,2-dimethylpropyl | —H | 2-chloro-4-fluorophenyl | —Cl | —Cl | A | 5.25 | |
| 355 | —NH—CH₂—CH₂—CH₂—CH₂— | * | 2-chloro-6-fluorophenyl | —Cl | —CN | | 3.66 | paste |
| 356 | —NH₂ | i-butyl | 2-chloro-6-fluorophenyl | —Cl | —CN | | 3.9 | oil |
| 357 | —CH₂—CF₃ | —H | 2-trifluoromethylphenyl | —Cl | —CN | | 3.44 | |
| 358 | 1,2-dimethylpropyl | —H | 2-trifluoromethylphenyl | —Cl | —Cl | A | 5.04 | |
| 359 | 1,2-dimethylpropyl | —H | 2-trifluoromethylphenyl | —Cl | —Cl | B | 5.05 | |
| 360 | —CH₂—C(CH₃)₃ | —H | 2-chlorophenyl | —Cl | —Cl | | 5.34 | |
| 361 | —CH₂—C(CH₃)₃ | —H | 2-chloro-4-fluorophenyl | —Cl | —Cl | | 5.4 | |
| 362 | —CH₂—CH₂—S—CH₂—CH₂— | * | 2-chloro-6-fluorophenyl | —Cl | —Cl | | 4.79 | |
| 363 | —CH₂—CH₂—S—CH₂—CH₂— | * | 2-trifluoromethylphenyl | —Cl | —Cl | | 4.68 | |
| 364 | —NH—CH₂—CH₂—CH₂—CH₂— | * | 2-trifluoromethylphenyl | —Cl | —Cl | | 4.41 | |
| 365 | —CH₂—CH₂—O—CH₂—CH₂— | * | 2-trifluoromethylphenyl | —Cl | —Cl | | 3.9 | |
| 366 | 2,2,2-trifluoro-1- | —H | 2-trifluoromethylphenyl | —Cl | —Cl | B | 4.49 | |
| | methylethyl | | | | | | | |
| 367 | 2,2,2-trifluoro-1- | —H | 2-trifluoromethylphenyl | —Cl | —Cl | A | 4.46 | |
| | methylethyl | | | | | | | |
| 368 | —NH—CH₂—CH₂—CH₂—CH₂— | * | 2-chloro-4-fluorophenyl | —Cl | —CN | | 3.78 | 209-11 |
| 369 | 2,2,2-trifluoro-1- | —H | 2-trifluoromethylphenyl | —Cl | —CN | A | 3.75 | |
| | methylethyl | | | | | | | |
| 370 | 2,2,2-influoro-1- | —H | 2-trifluoromethylphenyl | —Cl | —CN | B | 3.79 | |
| | methylethyl | | | | | | | |
| 371 | 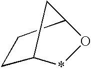 | * | 2-trifluoromethylphenyl | —Cl | —Cl | | 4.17 | |
| 372 | —NH₂ | i-butyl | 2-chloro-4-fluorophenyl | —Cl | —CN | | 4.09 | paste |
| 373 | —CH₂—CH₂—CH(CH₃)—CH₂—CH₂— | * | 2-chloro-6-fluorophenyl | —Cl | —Cl | | 5.77 | |
| 374 | —NH—CH₂—CH₂—CH₂—CH₂ | * | 2-chloro-6-fluorophenyl | —Cl | —Cl | | 4.4 | |
| 375 | —NH₂ | i-butyl | 2-chloro-6-fluorophenyl | —H | —Cl | | 4.11 | oil |
| 376 | 1,2-dimethylpropyl | —H | 2-trifluoromethylphenyl | —Cl | —CN | | 4.26 | |
| 377 | —NH₂ | i-butyl | 2-trifluoromethylphenyl | —Cl | —CN | | 3.96 | 128-30 |
| 378 | 2,2,2-trifluoro-1- | —H | 2-trifluoromethylphenyl | —Cl | —CN | A | 3.79 | |
| | methylethyl | | | | | | | |
| 379 | 2,2,2-trifluoro-1- | —H | 2-trifluoromethylphenyl | —Cl | —CN | B | 3.79 | |
| | methylethyl | | | | | | | |
| 380 | 2,2,2-trifluoro-1- | —H | 2,4,6-trifluorophenyl | —Cl | —Cl | | 4.42 | |
| | methylethyl | | | | | | | |
| 381 | —CH₂—CH₂—CH₂—NH— | * | 2-chloro-6-fluorophenyl | —Cl | —CN | | 3.23 | |
| 382 | —CH(CH₃)—C(CH₃)₃ | —H | 2-chloro-4-fluorophenyl | —Cl | —CN | | 4.78 | 180-5 |
| 383 | —CH(CH₃)—C(CH₃)₃ | —H | 2-chloro-6-flfuorphenyl | —Cl | —CN | | 4.6 | |
| 384 | —CH(CH₃)—C(CH₃)₃ | —H | 2,4,6-trifluorophenyl | —Cl | —CN | | 4.51 | |
| 385 | —CH₂—CH₂—O—CH₂—CH₂ | * | 2-chloro-5-nitrophenyl | —Cl | —NO₂ | | 2.91 | |
| 386 | 1,2,2-trimethylpropyl | —H | 2-chloro-6-fluorophenyl | —Cl | —CN | | 4.59 | 146-8 |
| 387 | 1,2,2-trimethylpropyl | —H | 2-chloro-6-fluorophenyl | —Cl | —CN | | 4.66 | 145-8 |
| 388 | —CH(CH₃—C(CH₃)₃ | —H | 2-trifluoromethylphenyl | —Cl | —CN | | 4.57 | |
| 389 | —CH(CH₃)—C(CH₃)₃ | —H | 2-chloro-4-fluorophenyl | —Cl | —Cl | | 5.63 | |
| 390 | 1,2,2-trimethylpropyl | —H | 2-chloro-4-fluorophenyl | —Cl | —CN | | 4.8 | paste |
| 391 | 1,2,2-trimethylpropyl | —H | 2-chloro-4-fluorophenyl | —Cl | —CN | | 4.83 | paste |
| 392 | —CH₂—CH₂—O—CH₂—CH₂— | * | 2-chlorophenyl | —Cl | —Br | | 3.97 | |
| 393 | —CH(CH₃)—C(CH₃)₃ | —H | 2-chloro-4-fluorophenyl | —Cl | —CSNH₂ | | 4.74 | paste |
| 394 | 1,2,2-trimethylpropyl | —H | 2,4,6-trifluorophenyl | —Cl | —CN | | 4.55 | |
| 395 | i-butyl | —H | 2-chloro-4-methoxyphenyl | —Cl | —CN | | 4.01 | |
| 396 | —CH(CH₃)—C(CH₃)₃ | —H | 2-chloro-4-methoxyphenyl | —Cl | —CN | | 4.67 | |
| 397 | —CH₂—C(CH₃)₃ | —H | 2-chloro-4-methoxyphenyl | —Cl | —CN | | 4.45 | |
| 398 | 1,2-dimethylpropyl | —H | 2-chlorophenyl | —Cl | —Br | | 5.31 | |
| 399 | —CH₂—CH₂—O—CH₂—CH₂ | * | 2-chlorophenyl | —Cl | I | | 4.13 | |
| 400 | 1,2-dimethylpropyl | —H | 2-chlorophenyl | —Cl | I | | 5.43 | |
| 401 | —CH(CH₃)—C(CH₃)₃ | —H | 2-chloro-4-fluorophenyl | —Cl | —CHO | | 4.43 | |
| 402 | —CH(CH₃)—C(CH₃)₃ | —H | 2-chloro-4-fluorophenyl | —Cl | —CH═N—O— | A + B | 5.48 | paste |
| | | | | | CH₃ | | | |
| 403 | —CH(CH₃)—C(CH₃)₃ | —H | 2-chloro-4-fluorophenyl | —Cl | —CH═N—O— | A | 5.5 | paste |
| | | | | | CH₃ | | | |
| 404 | —CH(CH₃)—C(CH₃)₃ | —H | 2-chloro-4-fluorophenyl | —Cl | —CH═N—O— | B | 5.57 | paste |
| | | | | | CH₃ | | | |
| 405 | 1,2-dimethylpropyl | —H | 2-chloro-4-methoxyphenyl | —Cl | —CN | | 4.33 | |
| 406 | —CH₂—CH₂—CH₂—CH₂—CH(CH₃)— | * | 2-chloro-4-methoxyphenyl | —Cl | —CN | | 4.65 | |
| 407 | 1,2,2-trimethylpropyl | —H | 2,4,6-trifluorophenyl | —Cl | cyclo- | | 5.88 | |
| | | | | | propyl | | | |
| 408 | —CH₂—CF₃ | —H | 2-chloro-4-methylphenyl | —Cl | —CN | | 3.47 | |
| 409 | 1,2-dimethylpropyl | —H | 2-chlorophenyl | —Cl | —CHO | | 3.98 | |
| 410 | i-butyl | —CH₃ | 2-chloro-4-methoxypyhenyl | —Cl | —CN | | 4.5 | |
| 411 | —CH₂—CH₂—O—CH₂—CH₂— | * | 2-chloro-4-methoxypyhenyl | —Cl | —CN | | 3.26 | |
| 412 | —CH₂—CH₂—CH₂—CH₂—CH(CH₃)— | * | 2-chloro-4-methoxypyhenyl | —Cl | —CN | | 4.18 | |
| 413 | —CH₂—CH₂—CH(CN)—CH₂—CH₂ | * | 2-chloro-4-fluorophenyl | —Cl | —Cl | | 4.06 | |
| 414 | 1,2-dimethylpropyl | —H | 2,4,6-trifluorophenyl | —Cl | cyclo- | | 5.5 | |
| | | | | | propyl | | | |
| 415 | —CH(CH₃—C(CH₃)₃ | —H | 2-chloro-6-fluorophenyl | —Cl | —CSNH₂ | | 4.52 | |
| 416 | 1,2-dimethylpropyl | —H | 2-chlorophenyl | —Cl | cyclo- | | 5.61 | |
| | | | | | propyl | | | |
| 417 | 1,2-dimethylpropyl | —H | 2,4,6-trifluorophenyl | —Cl | —Br | | 5.13 | |
| 418 | —CH₂—CH₂—CH(CH₃)—CH₂—CH₂ | * | 2,4,6-trifluorophenyl | —Cl | —Br | | 5.65 | |
| 419 | —CH₂—CH₂—O—CH₂—CH₂— | * | 2,4,6-trifluorophenyl | —Cl | —Br | | 3.97 | |
| 420 | 1,2,2-trimethylpropyl | —H | 2-chlorophenyl | —Cl | cyclo- | | 5.97 | |
| | | | | | propyl | | | |
| 421 | 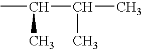 | —H | 2-chloro-4-fluorophenyl | —Cl | —CH₃ | | 4.79 | |
| 422 | 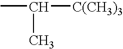 | —H | 2-chloro-4-fluorophenyl | —Cl | —CHO | | 4.43 | |
| 423 | 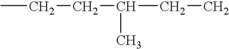 | * | 2-chlorophenyl | —Cl | —CHO | | 4.42 | |
| 424 | 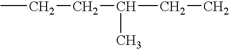 | * | 2-chloro-4-fluorophenyl | —Cl | —CHO | | 4.53 | |
| 425 | 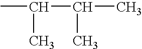 | H | 2-chlorophenyl | —Cl | —CHO | | 3.98 | |
| 426 | —CH₂—CH₂—O—CH₂—CH₂— | * | 2,4,6-trifluorophenyl | —Cl | —CHO | | 2.78 | |
| 427 | 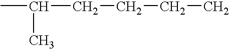 | * | 2,4,6-trifluorophenyl | —Cl | —CHO | | 4.24 | |
| 428 | 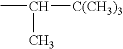 | H | 2-chloro-6-fluorophenyl | —Cl | —CHO | | 4.25 | |
| 429 | —NH—CH₂—CH₂—CH₂—CH₂ | * | 2,4,6-trifluorophenyl | —Cl | —CHO | | 3.28 | |
| 430 | 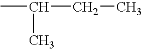 | H | 2,4,6-trifluorophenyl | —Cl | —CHO | | 3.52 | |
| 431 | 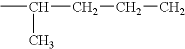 | * | 2,4,6-trifluorophenyl | —Cl | —CHO | | 3.74 | |
| 432 | —CH₂—CH₂—S—CH₂—CH₂— | * | 2,4,6-trifluorophenyl | —Cl | —CHO | | 3.45 | |
| 433 | 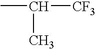 | H | 2,4,6-trifluorophenyl | —Cl | —CHO | | 3.39 | |
| 434 | —CH₂—CH₂—O—CH₂—CH₂— | * | 2-chloro-4-fluorophenyl | —Cl | —CHO | | 2.94 | |
| 435 | —CH₂—CH₂—S—CH₂—CH₂— | * | 2-chlorophenyl | —Cl | —CHO | | 3.51 | |
| 436 | 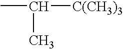 | H | 2,4,6-trifluorophenyl | —Cl | —CHO | | 3.16 | |
| 437 | 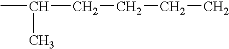 | * | 2-chlorophenyl | —Cl | —CHO | | 3.80 | |
| 438 | —CH₂—CH₂—S—CH₂—CH₂— | * | 2-chloro-4-fluor-phenyl | —Cl | —CHO | | 3.65 | |
| 439 | 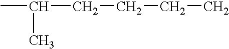 | * | 2-chloro-4-fluorophenyl | —Cl | —CHO | | 4.45 | |
| 440 | 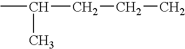 | * | 2-chloro-4-fluorophenyl | —Cl | —CHO | | 3.95 | |
| # denotes the point of attachment | | | | | | | | |
| The logP values were determined in accordance with EEC Directive 79/831 Annex V. A8 by HPLC (gradient method, acetonitrile/0.1% aqueous phosphoric acid) | | | | | | | | |
| *) means that R¹ and R² together with the nitrogen atom to which they are attached form a heterocyclic ring. | | | | | | | | |
| **) Some of the products were isolated as stereoisomers. “S” and “R” mean S configuration and R configuration, respectively, at the centre of chirality; “AS” means an unambiguous but unknown configuration at the centre of atropy and S configuration at the centre of chirality. BS means the respective other unambiguous but unknown configuration at the centre of atropy and S configuration at the centre of chirality. | | | | | | | | |
| # In turn, “AR” and “BR” mean the respective complementary configurations at the centre of atrophy, combined with the R configuration at the centre of chirality. Accordingly, in the case of identical substituents, “AR” and “BS”, and “AS” and “BR” are in each case pairs of enantiomers. | | | | | | | | |

### Preparation of the Compound of Example 401

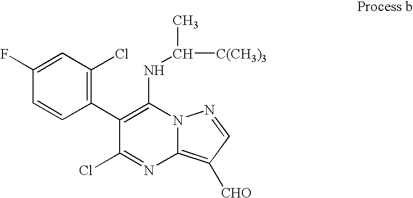

Under an atmosphere of argon and stirring at −50° C., a 1-molar solution of 649 mg (0.812 mmol) of diisobutylaluminium hydride in toluene is slowly added dropwise to a solution of 300 mg (0.738 mmol) of 3-cyano-5-chloro-6-(2-chloro-4-fluorophenyl)-7-(1,2,2-trimethylpropylamino)pyrazolo[1,5a]pyrimidine in 13.2 g of dichloromethane. After the addition has ended, stirring at −50° C. is continued for another 30 minutes. The temperature of the reaction mixture is then allowed to increase to 0° C., saturated aqueous ammonium chloride solution is added and the mixture is stirred at 0° C. for 2 hours. 1 N hydrochloric acid is added, the organic phase is separated off and the aqueous phase is extracted three more times with dichloromethane. The combined organic phases are washed successively with saturated aqueous sodium bicarbonate solution and with saturated aqueous sodium chloride solution, dried over sodium sulphate and then concentrated under reduced pressure. In this manner, 300 mg (99% of theory) of 3-formyl-5-chloro-6-(2-chloro-4-fluorophenyl)-7-(1,2,2-trimethylpropyl amino)pyrazolo[1,5a]pyrimidine are obtained.

HPLC: log P=4.43.

### Preparation of the Compound According to Example 402

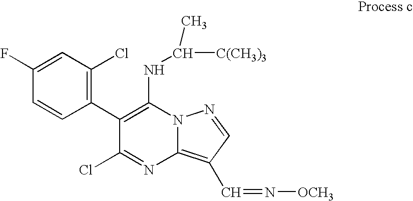

At room temperature, 73 mg (0.880 mmol) of methoxyamine hydrochloride and 1.0 g of the weak basic ion exchanger commercially available under the name Amberlyst A-21 are added to a solution of 300 mg (0.733 mmol) of 3-formyl-5-chloro-6-(2-chloro-4-fluorophenyl)-7-(1,2,2-trimethylpropylamino)pyrazlo[1,5a]pyrimidine in 20 ml of ethanol, and the mixture is shaken at room temperature for 18 hours. The reaction mixture is then filtered, and the filtrate is concentrated under reduced pressure. This gives 220 mg of 3-methoximino-5-chloro-6-(2-chloro-4-fluorophenyl)-7-(1,2,2-trimethylpropylamino)pyrazolo[1,5a]pyrimidine in the form of a mixture of atropisomers.

HPLC: log P=5.48.

### Preparation of Intermediates of the Formula (II)

### Example 441

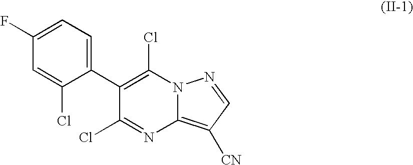 Process (g):

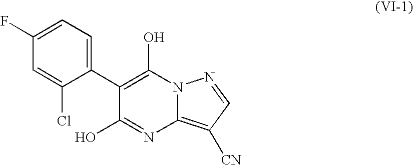

48 g (0.184 mol) of dimethyl 2-chloro-4-fluorophenylmalonate are mixed with 19.91 g (0.184 mol) of 4-cyano-5-aminopyrazole and with 37.55 g (0.203 mol) of tri-n-butylamine, and the mixture is stirred at 180° C. for 6 hours. The methanol formed during the reaction is distilled off. The reaction mixture is then cooled to room temperature. At 95° C. and 1 mbar, volatile components are distilled off. The residue obtained is 6-(2-chloro-4-fluorophenyl)-5,7-dihydroxypyrazolo[1,5-a]pyrimidine-3-carbonitrile in the form of a crude product which is used for further synthesis without additional purification.
Process (e)

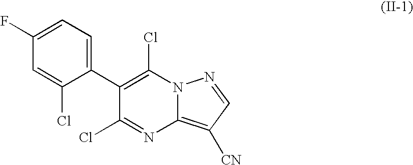

The 6-(2-chloro-4-fluorophenyl)-5,7-dihydroxypyrazolo[1,5-a]pyrimidine-3-carbo-nitrile obtained above is, in crude form, dissolved in 367.3 g (2.395 mol) of phosphorus oxychloride. At room temperature, 31.95 g (0.153 mol) of phosphorus pentachloride is added a little at a time. The mixture is then boiled under reflux for 12 hours. The volatile components are distilled off under reduced pressure, dichloromethane is added to the residue and the mixture is washed with water. The organic phase is dried over sodium sulphate and concentrated under reduced pressure. The residue is chromatographed on silica gel using 3 parts of cyclohexane and 1 part of ethyl acetate as mobile phase. This gives 21 g of 95.7% pure 3-cyano-5,7-dichloro-6-(2-chloro-4-fluorophenyl)pyrazolo[1,5-a]pyrimidine.

HPLC: log P=3.48

¹H-NMR (DMSO-d6, tetramethylsilane): δ=7.44-7.52 (1H); 7.62-7.66 (1H); 7.71-7.77 (1H); 9.03 (1H) ppm.

### Example 442

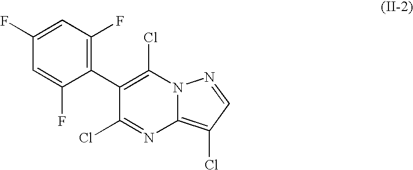 Process (e)

26 g (82.4 mmol) of 3-chloro-6-(2,4,6-trifluorophenyl)pyrazolo[1,5-a]pyrimidine-5,7-diol and 8.6 g (41.2 mmol) of phosphorus pentachloride are stirred in 126.3 g of phosphorus oxychloride at 110° C. for one hour. After cooling, the reaction mixture is stirred with water and dichloromethane, with ice-cooling. The organic phase is separated off, dried and concentrated under reduced pressure. The residue is chromatographed on silica gel using methyl t-butyl ether/petroleum ether (1:9). This gives 5 g (16.4% of theory) of 3,5,7-trichloro-6-(2,4,6-trifluorophenyl)pyrazolo[1,5-a]pyrimidine.

HPLC: log P=3.97

### Example 443

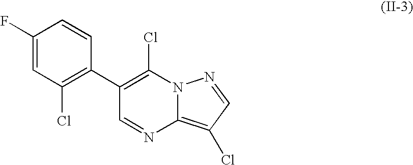 Process (d):

14.2 g (11.9 mmol) of 25% pure 3-chloro-6-(2-chloro-4-fluorophenyl)pyrazolo[1,5-a]pyrimidin-7-ol and 1.24 g (5.9 mmol) of phosphorus pentachloride are stirred in 16.3 g of phosphorus oxychloride at 110° C. for one hour and then for4 hours without further heating. After cooling, the reaction mixture is stirred with water and dichloromethane, with ice-cooling. The organic phase is separated off, dried and concentrated under reduced pressure. The residue is chromatographed on silica gel using n-hexane/ethyl acetate (3:1 to 1:1). This gives 2.1 g (54% of theory) of 3,7-dichloro-6-(2-chloro-4-fluorophenyl)pyrazolo[1,5-a]pyrimidine.

HPLC: log P=3.56

The compounds of the formula

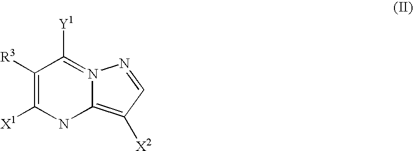

listed in Table 2 below are prepared by the methods described above.

TABLE 2

| Ex. No. | X¹ | Y¹ | R³ | X² | logP | m.p.: (° C.) |
|---|---|---|---|---|---|---|
| 444 | —Cl | —Cl | 2-chloro-6-fluorophenyl | —CN | 3.31 | |
| 445 | —Cl | —Cl | 2-chloro-4-fluorophenyl | —Cl | | |
| 446 | —Cl | —Cl | 2,4-difluorophenyl | —CN | 3.16 | 136-38 |
| 447 | —Cl | —Cl | 2,6-dichlorophenyl | —CN | 3.59 | |
| 448 | —Cl | —Cl | 2,4,6-trifluorophenyl | —CN | 3.2 | |
| 449 | —H | —Cl | 2-chloro-6-fluorophenyl | —CN | | |
| 450 | H | —Cl | 2-chloro-6-fluorophenyl | —Cl | | |
| 451 | —Cl | —Cl | 2,4,6-trifluorophenyl | 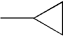 | 4.38 | |
| 452 | —Cl | —Cl | 2-chlorophenyl | 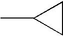 | | |

The logP values were determined in accordance with EEC Directive 79/831 Annex V.A8 by HPLC (gradient method, acetonitrile/0.1% aqueous phosphoric acid)

### Preparation of Intermediates of the Formulae (V) and (VI)

### Example 453

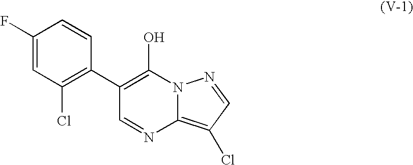 Process (f)

11.3 g (43.85 mmol) of methyl 2-(2-chloro-4-fluorophenyl)-3-(dimethylamino)-2-acrylate and 5.2 g (43.85 mmol) of 4-chloro-1H-pyrazole-5-amine are stirred in 11.5 ml of tri-n-butylamine at 180° C. for 6 hours, and the methanol and dimethylamine formed are distilled off. The mixture is then concentrated further under reduced pressure. This gives 14.2 g (27% of theory) of 25% pure 3-chloro-6-(2-chloro-4-fluorophenyl)pyrazolo[1,5-a]pyrimidin-7-ol.

### Example 454

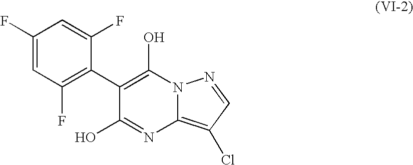 Process (g)

11.15 g (42.5 mmol) of dimethyl 2-(2,4,6-trifluorophenyl)malonate and 5 g (42.5 mmol) of 4-chloro-1H-pyrazole-5-amine are stirred in 11.5 ml of tri-n-butylamine at 180° C. for 3 hours, and the methanol formed is distilled off. The product is decanted off. This gives 21 g (76% of theory) of 49% pure 3-chloro-6-(2,4,6-trifluorophenyl)pyrazolo[1,5-a]pyrimidine-5,7-diol.

The compounds of the formula

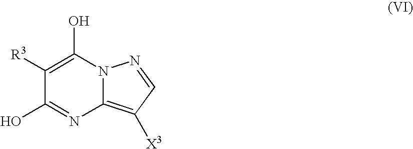

listed in Table 3 below are also prepared by the methods described above.

TABLE 3

| Ex. No. | R³ | X³ | log P |
|---|---|---|---|
| 455 | 2,4,6-trifluorophenyl | 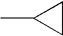 | |
| 456 | 2-chlorophenyl | 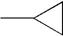 | |
| 457 | 2-chloro-4-fluorophenyl | —CH₃ | |

### Preparation of Amines of the Formula (III)

### Example 458

Process (h), First Step:

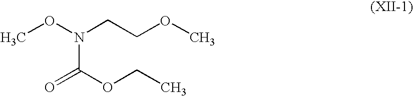

1000 mg of ethyl N-methoxycarbamate are initially charged in 10.0 ml of dimethylformamide and 403 mg of sodium hydride are added a little at a time and the temperature was maintained by cooling at 30° C. The reaction mixture is stirred at 30° C. for 2 hours, and 3500 mg of 2-bromoethyl methyl ether are then added. The reaction mixture is stirred at 20° C.-25° C. for 18 hours and then stirred into 20 ml of water. The resulting reaction mixture is concentrated to dryness under reduced pressure and extracted four times with in each case 30 ml of dichloromethane. The organic extracts are dried over sodium sulphate, filtered and concentrated to dryness under reduced pressure.

This gives 1200 mg of ethyl (N-methoxy-N-methoxyethyl)carbamate (purity 77.6%, yield 62.6%).
Process (h), Second Step:

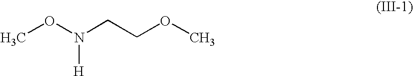

200 mg of ethyl (N-methoxy-N-methoxyethyl)carbamate are initially charged in 4.0 ml of aqueous ethanol (59%), 240.6 mg of potassium hydroxide are added and the mixture is stirred at 40° C. for 18 hours. The reaction mixture is then stirred into 50 ml of water and extracted three times with in each case 20 ml of diethyl ether and three times with in each case 20 ml of dichloromethane. The combined organic phases are washed twice with in each case 20 ml of water, dried and, at 20° C. and under reduced pressure, concentrated to a volume of 20 ml.

With ice-cooling, 2 ml of hydrochloric acid are added to the resulting solution and the mixture is stirred at room temperature for 1 hour and, at 20° C. and under reduced pressure, concentrated to dryness.

The resulting product is digested three times with in each case 15 ml of methanol and then, at 20° C. and under reduced pressure, concentrated to dryness.

This gives 140 mg of N-methoxy-N-methoxyethylamine hydrochloride (yield 87.6%).

### Example 459

Process (i), First Step:

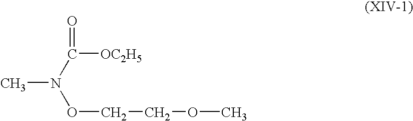

A mixture of 1000 mg of ethyl N-hydroxy-N-methylcarbamate and 1166 mg of 2-bromoethyl methyl ether is heated with stirring to reflux temperature, and a solution of 493 mg of potassium hydroxide in 5 ml of ethanol is then added dropwise. The reaction mixture is boiled under reflux for 10 hours and then worked up by filtering the reaction mixture and concentrating the filtrate under reduced pressure. A mixture of water and ethyl acetate is added to the residue that remains. The organic phase is separated off and washed with saturated aqueous ammonium chloride solution and then with water. The organic phase is then dried over sodium sulphate and concentrated under reduced pressure. This gives 0.7 g of a product which, according to gas chromatography, consists to 83% of ethyl (N-methyl-N-methoxyethoxy)carbamate. Accordingly, the calculated yield is 39% of theory.
Process (i), Second Step:

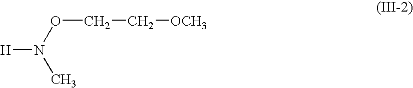

240.6 mg of powdered potassium hydroxide are added to a mixture of 200 mg of ethyl(N-methyl-N-methoxyethoxy)carbamate, 4 ml of ethanol and 4 ml of water, and the mixture is stirred at 40° C. for 2 hours. The reaction mixture is then stirred into 50 ml of water, and then extracted three times with in each case 20 ml of diethyl ether and subsequently three times with in each case 20 ml of methylene chloride. The combined organic phases are washed twice with in each case 20 ml of water, dried over sodium sulphate and, at room temperature and under reduced pressure, concentrated to a volume of 20 ml. With ice-cooling, 1 ml of ethereal hydrochloric acid is added to the resulting solution. The crystals that precipitate out are filtered off and dried. In this manner, 190 mg of N-methyl-N-methoxyethoxyamine hydrochloride are obtained.

### Example 460

Process (i), First Step:

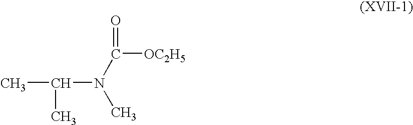

At room temperature and with stirring, 475 mg of sodium hydride are added to a mixture of 2000 mg of ethyl N-(2,2,2-trifluoro-1-methylethyl)carbamate and 20 ml of tetrahydrofuran. A solution of 4600 mg of iodomethane in 10 ml of tetrahydrofuran is then added dropwise with stirring and at room temperature. The reaction mixture is stirred at 50° C. for 16 hours, and water is then added. The mixture is extracted three times with in each case 20 ml of methylene chloride and the combined organic phases are dried over sodium sulphate and concentrated under reduced pressure. This gives 1000 mg of a product which, according to gas chromatography, consists to 75% of ethyl N-(2,2,2-trifluoro-1-methylethyl)-N-methylcarbamate. Accordingly, the calculated yield is 34.86%.
Process (j), Second Step:

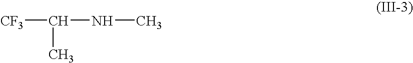

1070 mg of powdered potassium hydroxide are added to a mixture of 1000 mg of ethyl N-(2,2,2-trifluoro-1-methylethyl)-N-methylcarbamate, 20 ml of ethanol and 20 ml of water, and the mixture is stirred at 40° C. for 66 hours. The reaction mixture is then diluted with water and extracted three times with in each case 20 ml of a mixture of identical parts of methylene chloride and diethyl ether. The combined organic phases are dried over sodium sulphate and then concentrated at room temperature and under slightly reduced pressure. With ice-cooling, ethereal hydrochloric acid is added to the resulting solution and the mixture is stirred at room temperature for 60 hours. Concentration under reduced pressure gives 280 mg of N-(2,2,2-trifluoro-1-methylethyl)-N-methylamine hydrochloride. Accordingly, the calculated yield is 34% of theory.

### Example 461

Process (k):

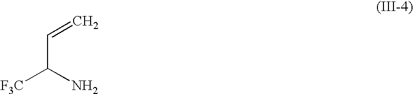

600 mg of benzyl N-(1-trifluoromethyl-2-propene)carbamate in 8.0 ml of 16% strength hydrochloric acid are heated under reflux for 1.5 hours. After cooling to 20° C., the mixture is extracted twice with in each case 20 ml of diethyl ether.

The aqueous phase that remains is concentrated to dryness under reduced pressure and three portions of in each case 10 ml of methanol are added. The methanol is removed under reduced pressure and 310 mg of (1-trifluoromethylprop-2-ene)amine hydrochloride are isolated. Accordingly, the calculated yield is 82.9% of theory.

The carbamates listed in the tables below can also be prepared by the methods described above.

TABLE 4
(XII)

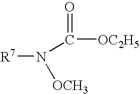

| Example | | | |
|---|---|---|---|
| No. | Comp. No. | R⁷ | logP |
| 462 | XII-2 | 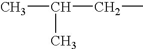 | 2.38 |
| 463 | XII-3 | 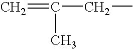 | 2.06 |

TABLE 5
(XIV)

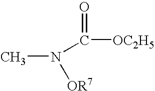

| Example | | | |
|---|---|---|---|
| No. | Comp. No. | R⁷ | Physical const. |
| 464 | XIV-2 | 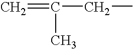 | |

TABLE 6
(XVII)

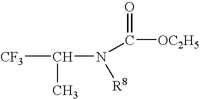

| Example | Comp. | | |
|---|---|---|---|
| No. | No. | R⁸ | Physical const. |
| 465 | XVII-2 | —C₂H₅ | ¹H-NMR (400 MHz, CD₃CN): |
| | | | δ (ppm) = 1.13 (t, CH₃CH₂N), 1.21 (t, |
| | | | CH₃CHCF₃), 1.23 (t, CH₃CH₂O), 3.20 |
| | | | (m, CH₂N, CHCF₃), 4.1 (q, CH₃CH₂O). |

The amines listed below can also be prepared by the methods described above.

TABLE 7
(III)

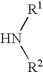

| Example | Comp. | | | |
|---|---|---|---|---|
| No. | No. | R¹ | R² | Physical const. |
| 466 | III-5 | 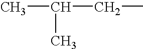 | —OCH₃ | ¹H-NMR (400 MHz, CD₃CN): δ (ppm) = 1.03 (d, CH₃)₂CH), 3.06 (d, CH₂), 3.28 (b, (CH₃)₂CH), 4.01 (s, OCH₃) |
| 467 | III-6 | 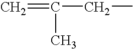 | —OCH₃ | ¹H-NMR (400 MHz, DMSO): δ (ppm) = 1.76 (s, CH₃(CCH₂)CH₂), 3.29 (b, NH, CH₃(CCH₂)CH₂, OCH₃), 7.89, 5.02 (2 s, CH₃(CCH₂)CH₂). |
| 468 | III-7 | 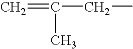 | | |
| 469 | III-8 | 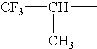 | —C₂H₅ | ¹H-NMR (400 MHz, DMSO): δ (ppm) = 1.06 (m, CH₃CH₂N, CH₃CHCF₃), 3.20 (m, CH₂N), 4.1 (m, CHCF₃). |

The amines listed in Examples 466 to 469 were in each case isolated and terized in the form of their hydrochlorides.

### Preparation of an Aminopyrazole

### Example 470

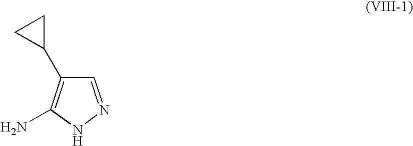

a) Under an atmosphere of argon and with stirring at room temperature, 400 ml of diethyl ether are added dropwise over a period of one hour to a mixture of 16.223 g (200 mmol) of cyclopropylacetonitrile and 15.556 g (210 mmol) of ethyl formate. 4.598 g (200 mmol) of sodium are added, and the mixture is stirred at room temperature for 4 days. Once the metallic sodium has dissolved, the mixture is cooled to 10° C. and 12.01 g (200 mmol) of acetic acid are added over a period of 30 minutes, the reaction mixture being maintained at temperatures between 10° C. and 15° C. The reaction mixture is stirred for another 15 minutes and then filtered off with suction, and the residue is washed with 30 ml of cold diethyl ether. The filtrate is concentrated under reduced pressure. This gives 22.0 g of 1-formyl-1-cyclopropylacetonitrile in the form of a crude product which is used without prior purification for the further synthesis.

b) With stirring at room temperature, a mixture of 8.670 g (0.173 mol) of hydrazine hydrate and 3.12 ml of acetic acid is introduced into a solution of 21.825 g (0.200 mol) of 1-formyl-1-cyclopropylacetonitrile in 20 ml of ethanol. The reaction mixture is stirred under reflux for 4 hours and then worked up by concentration under reduced pressure. This gives 13.7 g (55.6% of theory) of 4-cyclopropyl-1H-pyrazole-5-amine.

### Use Examples

### Example A

Venturia Test (Apple)/Protective

| | |
|---|---|
| Solvents: | 24.5 parts by weight of acetone |
| | 24.5 parts by weight of dimethylacetamide |
| Emulsifier: | 1.0 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous conidia suspension of the apple scab pathogen Venturia inaequalis and then remain in an incubation cabin at about 20° C. and 100% relative atmospheric humidity for 1 day.

The plants are then placed in a greenhouse at about 21° C. and a relative atmospheric humidity of about 90%.

Evaluation is carried out 10 days after the inoculation. 0% means an efficacy which corresponds to that of the control, whereas an efficacy of 100% means that no infection is observed.

Active compounds, active compound application rates and test results are shown in the table below.

TABLE A
Venturia test (apple)/protective

| | Application rate | Efficacy |
|---|---|---|
| Active compound | in g/ha | in % |
| According to the invention: | | |
| 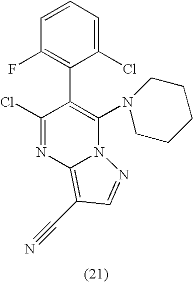 | 100 | |
| 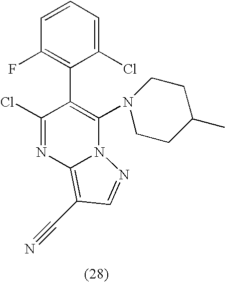 | 100 | 97 |
| 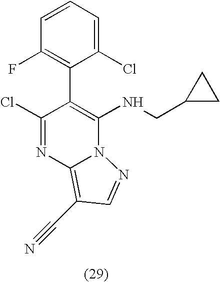 | 100 | 100 |
| 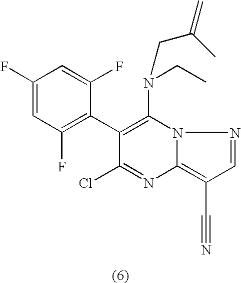 | 100 | 100 |
| 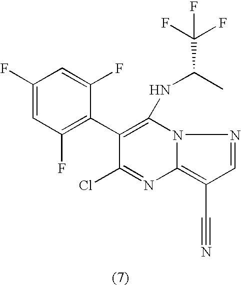 | 100 | 100 |
| 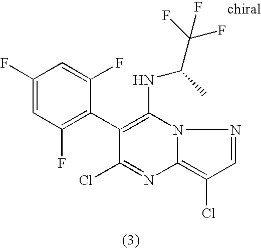 | 100 | 98 |
| 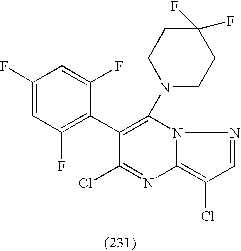 | 100 | 100 |
| 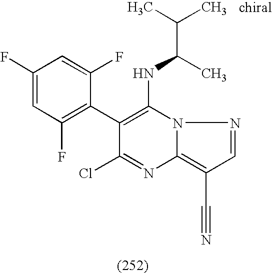 | 100 | 100 |
| 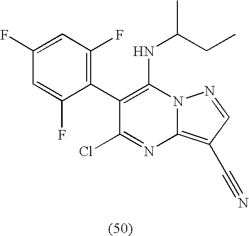 | 100 | 100 |
| 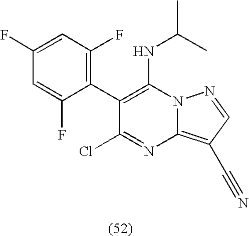 | 100 | 98 |
| 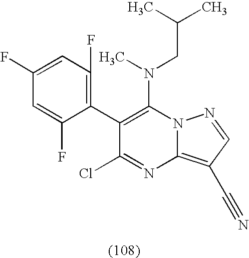 | 100 | 100 |
| 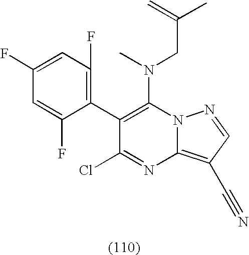 | 100 | 100 |
| 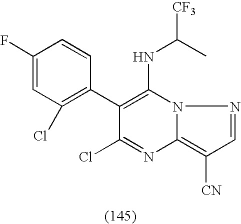 | 100 | 93 |
| 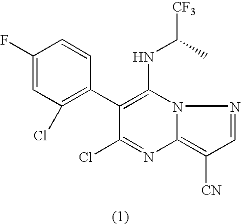 | 100 | 100 |
| 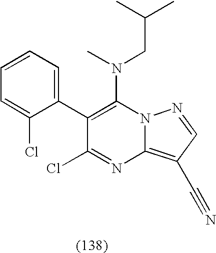 | 100 | 100 |
| 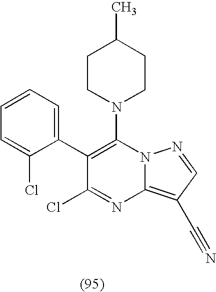 | 100 | 99 |
| 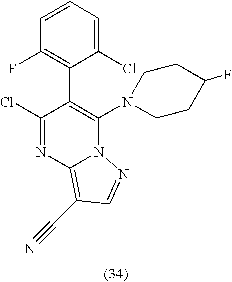 | 100 | 98 |
| 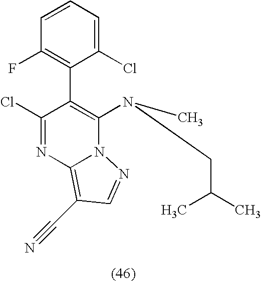 | 100 | 99 |
| 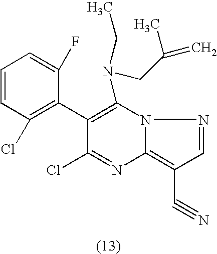 | 100 | 100 |
| 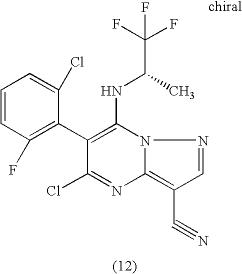 | 100 | 100 |

### Example B

Botrytis Test (Bean)/Protective

| | |
|---|---|
| Solvents: | 24.5 parts by weight of acetone |
| | 24.5 parts by weight of dimethylacetamide |
| Emulsifier: | 1.0 part by weight of alkylaryl polyglycol ether |

To produce a suitable preparation of active compound, 1 part by weight of active compound is mixed with the stated amounts of solvent and emulsifier, and the concentrate is diluted with water to the desired concentration. To test for protective activity, young plants are sprayed with the preparation of active compound at the stated application rate. After the spray coating has dried on, 2 small pieces of agar colonized by Botrytis cinerea are placed onto each leaf. The inoculated plants are placed in a dark chamber at about 20° C. and 100% relative atmospheric humidity.

2 days after the inoculation, the size of the infected areas on the leaves is evaluated. 0% means an efficacy which corresponds to that of the control, whereas an efficacy of 100% means that no infection is observed.

Active compounds, active compound application rates and test results are shown in the table below.

TABLE B
Botrytis test (bean)/protective

| | Active compound | |
|---|---|---|
| | application rate | Efficacy |
| Active compound | in g/ha | in % |
| According to the invention: | | |
| 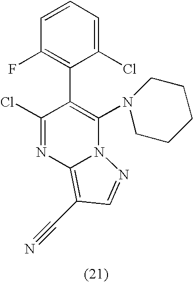 | 500 | 93 |
| 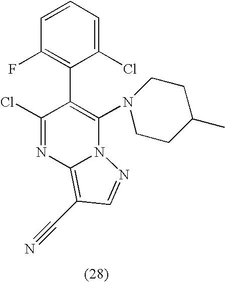 | 500 | 96 |
| 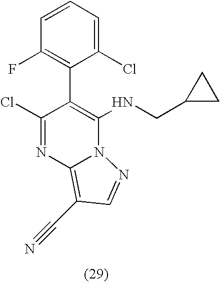 | 500 | 99 |
| 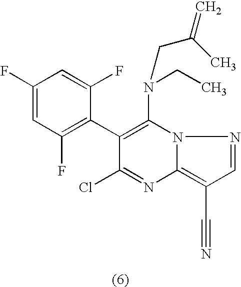 | 500 | 100 |
| 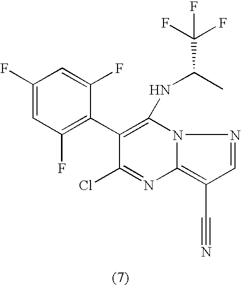 | 500 | 99 |
| 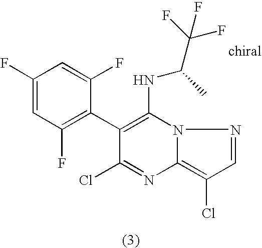 | 500 | 100 |
| 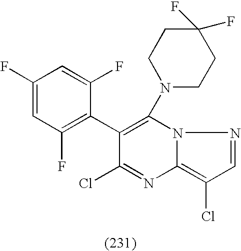 | 500 | 95 |
| 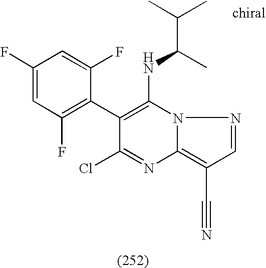 | 500 | 96 |
| 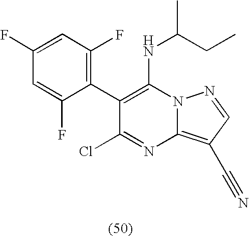 | 500 | 97 |
| 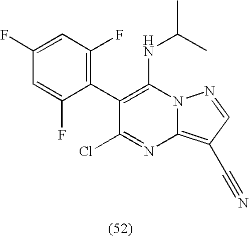 | 500 | 93 |
| 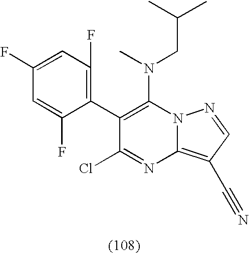 | 500 | 100 |
| 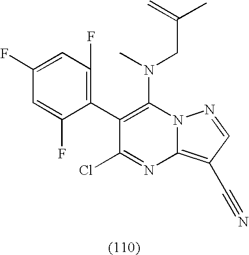 | 500 | 100 |
| 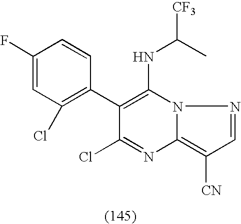 | 500 | 99 |
| 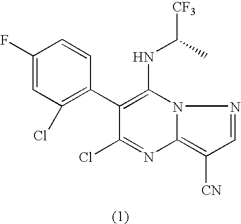 | 500 | 100 |
| 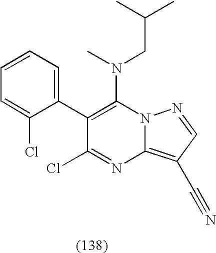 | 500 | 100 |
| 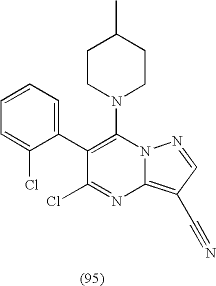 | 500 | 95 |
| 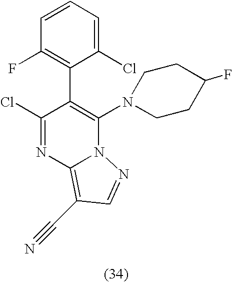 | 500 | 94 |
| 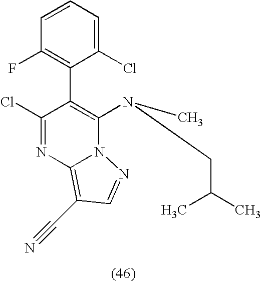 | 500 | 99 |
| 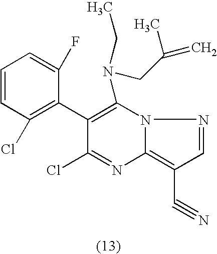 | 500 | 100 |
| 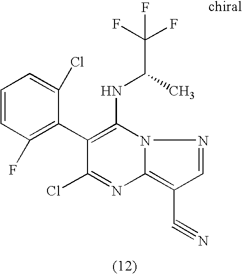 | 500 | 100 |

### Example C

Altemaria Test (Tomato)/Protective

| | |
|---|---|
| Solvent: | 49 parts by weight of N,N-dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young tomato plants are sprayed with the preparation of active compound at the stated application rate. 1 day after the treatment, the plants are inoculated with a spore suspension of Alternaria solani and then remain at 100% rel. humidity and 20° C. for 24 h. The plants then remain at 96% rel. atmospheric humidity and a temperature of 20° C.

Evaluation is carried out 7 days after the inoculation. 0% means an efficacy which corresponds to that of the control, whereas an efficacy of 100% means that no infection is observed.

Active compounds, active compound application rates and test results are shown in the table below.

TABLE C
Alternaria test (tomato)/protective

| | Active compound | |
|---|---|---|
| | application rate | Efficacy |
| Active compound | in g/ha | in % |
| According to the invention: | | |
| 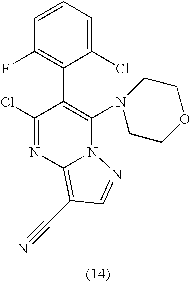 | 750 | 95 |
| 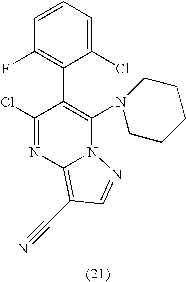 | 750 | 95 |
| 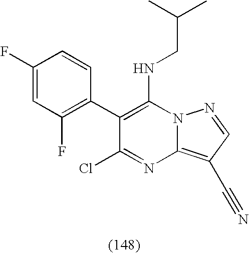 | 750 | 95 |
| 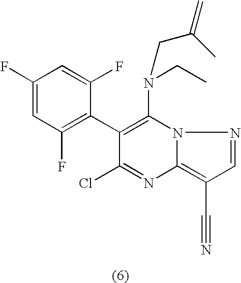 | 750 | 95 |
| 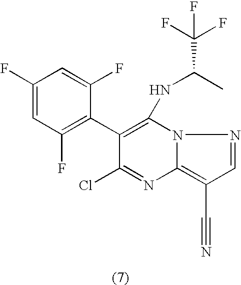 | 750 | 95 |
| 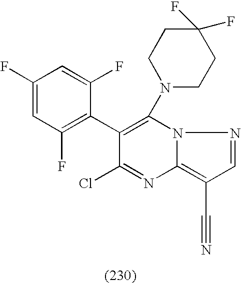 | 750 | 90 |

### Example D

Fusarium nivale (var. majus) Test (Wheat)/Protective

| | |
|---|---|
| Solvent: | 25 parts by weight of N,N-dimethylacetamide |
| Emulsifier: | 0.6 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are sprayed with a conidia suspension of Fusarium nivale (var. majus).

The plants are placed in a greenhouse under transparent incubation hoods at a temperature of about 15° C. and a relative atmospheric humidity of about 100%.

Evaluation is carried out 6 days after the inoculation. 0% means an efficacy which corresponds to that of the control, whereas an efficacy of 100% means that no infection is observed.

Active compounds, active compound application rates and test results are shown in the table below.

TABLE D
Fusarium nivale (var. majus) test (wheat)/protective

| | Active compound | |
|---|---|---|
| | application rates | Efficacy |
| Active compound | in g/ha | in % |
| According to the invention: | | |
| 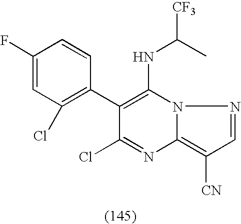 | 500 | 88 |
| 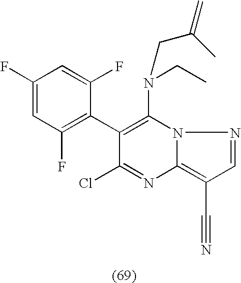 | 500 | 100 |
| 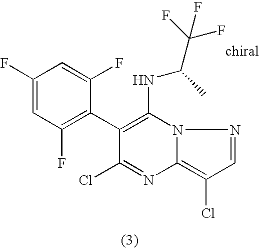 | 500 | 80 |

### Example E

Pyricularia Test (Rice)/Protective

| | |
|---|---|
| Solvent: | 25 parts by weight of N,N-dimethylacetamide |
| Emulsifier: | 0.6 part by weight of alkylaryl polyglycol ether |

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. After the spray coating has dried on, the plants are inoculated with an aqueous spore suspension of Pyricularia oryzae. The plants are then placed in a greenhouse at 100% relative atmospheric humidity and 25° C.

Evaluation is carried out 6 days after the inoculation. 0% means an efficacy which corresponds to that of the control, whereas an efficacy of 100% means that no infection is observed.

Active compounds, active compound application rates and test results are shown in the table below.

TABLE E
Pyricularia test (rice)/protective

| | Active compound | |
|---|---|---|
| | application rate | Efficacy |
| Active compound | in g/ha | in % |
| According to the invention: | | |
| 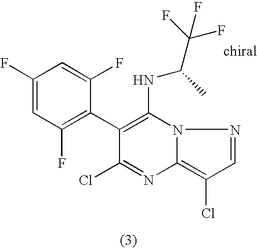 | 500 | 88 |
| 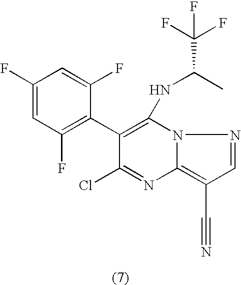 | 500 | 88 |
| 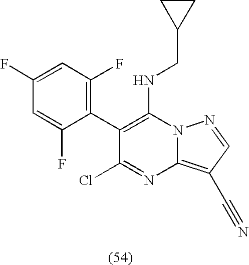 | 500 | 86 |
| 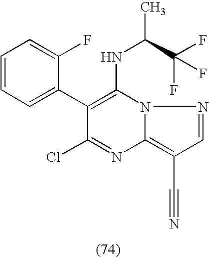 | 500 | 75 |

### Example F

Plutella Test

| | |
|---|---|
| Solvent: | 100 parts by weight of acetone |
| | 1900 parts by weight of methanol |

To produce a suitable preparation of active compound, 1 part by weight of active compound is mixed with the stated amount of solvent, and the concentrate is diluted with methanol to the desired concentrations.

A stated amount of preparation of active compound of the desired concentration is pipetted onto a standardized amount of synthetic feed. After the methanol has evaporated, about 200-300 eggs of the diamondback moth (Plutella xyostella) are placed onto the feed.

After the desired period of time, the kill of the eggs or larvae in % is determined. 100% means that all animals have been killed; 0% means that none of the animals has been killed.

Active compounds, active compound concentrations and test results are shown in the table below.

TABLE F
Plant-damaging insects
Plutella test

| | Concentration of | |
|---|---|---|
| | active compound | Kill rate in |
| Active compound | in ppm | % after 7^{d} |
| According to the invention: | | |
| 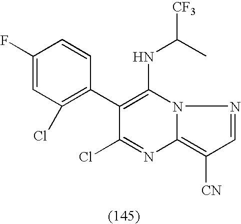 | 1000 | 100 |

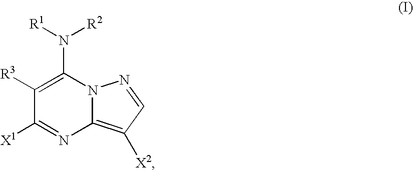

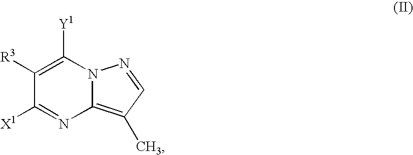

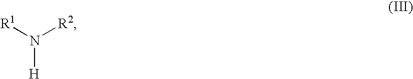

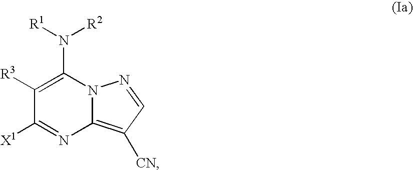

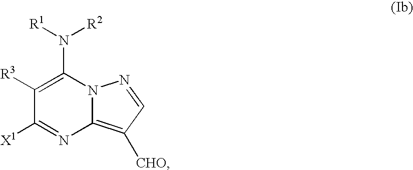

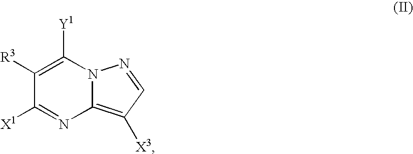

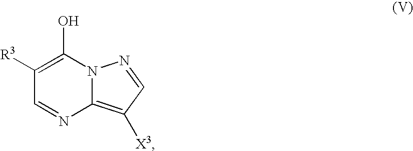

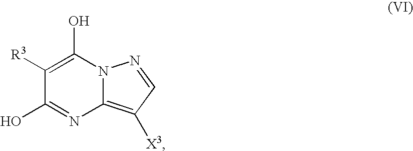

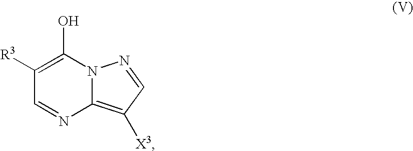

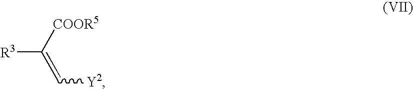

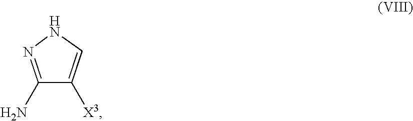

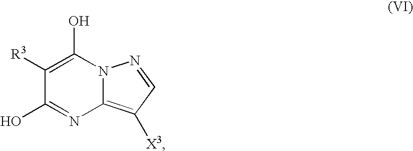

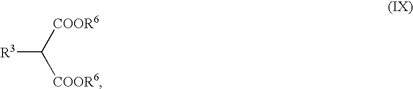

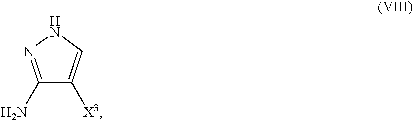

1-12. (canceled)

13. A pyrazolopyrimidine of formula (I) 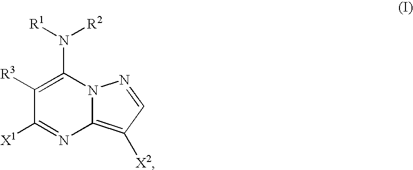
in which
R¹ represents amino or hydroxyl; or represents optionally substituted alkyl, alkenyl, alkynyl, cycloalkyl, alkoxy, alkenyloxy, alkynyloxy, cycloalkyloxy, alkylamino, dialkylamino, alkenylamino, alkynylamino, cycloalkylamino, N-cycloalkyl-N-alkylamino, alkylideneamino, or heterocyclyl,
R² represents hydrogen; or represents optionally substituted alkyl, alkenyl, alkynyl, or cycloalkyl, or
R¹ and R² together with the nitrogen atom to which they are attached form an optionally substituted heterocyclic ring,
R³ represents optionally substituted aryl,
X¹ represents hydrogen or halogen, and
X² represents halogen, cyano, nitro, alkyl, haloalkyl, cycloalkyl, formyl, thiocarbamoyl, alkoxycarbonyl, alkylcarbonyl, hydroximinoalkyl, or alkoximinoalkyl,
with the proviso that when R¹ represents amino, the pyrazolopyrimidine of formula (I) is optionally in the form of an acid addition salt.

14 A process for preparing pyrazolopyrimidines of formula (I) according to claim 13 comprising
(a) reacting a halopyrazolopyrimidine of formula (II) 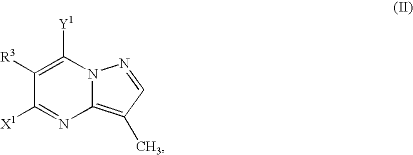
in which
R³ and X¹ are as defined for formula (I) in claim 13,
x³ represents halogen, cyano, nitro, alkyl, haloalkyl, cycloalkyl, thiocarbamoyl, alkoxycarbonyl, or alkylcarbonyl, and
Y¹ represents halogen,
with an amine of formula (III) 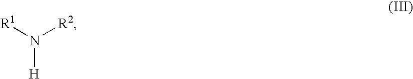
in which R¹ and R² are as defined for formula (I) in claim 13, optionally in the presence of a diluent, optionally in the presence of a catalyst, and optionally in the presence of an acid acceptor,
or
(b) reacting a pyrazolopyrimidine of formula (Ia) 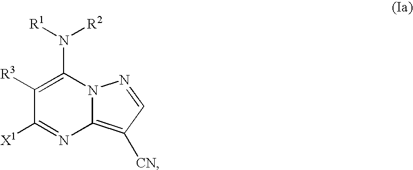
in which R¹, R², R³, and X¹ are as defined for formula (I) in claim 13, with a diisobutylaluminum hydride in the presence of aqueous ammonium chloride solution and in the presence of an organic diluent,
or
(c) reacting a pyrazolopyrimidine of formula (Ib) 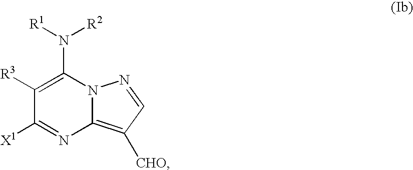
in which R¹, R², R³, and X¹ are as defined for formula (I) in claim 13, with an amino compound of formula (IV)
H₂N—OR⁴ (IV),
or an acid addition salt thereof,
in which R⁴ represents hydrogen or alkyl,
in the presence of a diluent and optionally in the presence of a catalyst, and when R¹ represents amino, optionally adding an acid to the resulting pyrazolopyrimidine of formula (I).

15. A composition for controlling harmful organisms comprising one or more pyrazolopyrimidines of formula (I) according to claim 13, or an acid addition salt thereof, and one or more extenders and/or surfactants.

16. A method for controlling harmful organisms comprising applying an effective amount of one or more pyrazolopyrimidines of formula (I) according to claim 13, or an acid addition salt thereof, the harmful organisms and/or their habitat.

17. A process for preparing a composition for controlling harmful organisms comprising mixing one or more pyrazolopyrimidines of formula (I) according to claim 13, or an acid addition salt thereof, with one or more extenders and/or surfactants.

18. A halopyrazolopyrimidine of formula (II) 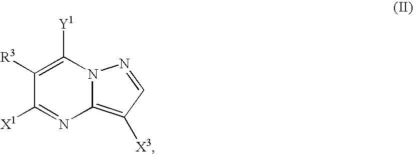
in which
R³ represents optionally substituted aryl,
X¹ represents hydrogen or halogen,
X¹ represents halogen, cyano, nitro, alkyl, thiocarbamoyl, cycloalkyl, haloalkyl, alkoxycarbonyl, or alkylcarbonyl, and
Y¹ represents halogen.

19. A process for preparing a halopyrazolopyrimidine of formula (II) according to claim 18 comprising
(d) reacting a hydroxypyrazolopyrimidine of formula (V) 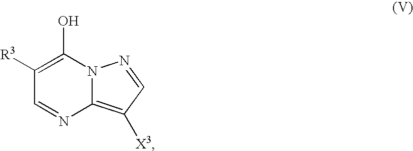
in which R³ and X³ are as defined for formula (II) in claim 18, with a halogenating agent, optionally in the presence of a diluent,
or
(e) reacting a dihydroxypyrazolopyrimidine of formula (VI) 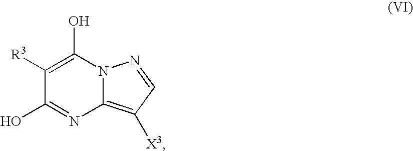
in which R³ and X³ are as defined for formula (II) in claim 18, with a halogenating agent, optionally in the presence of a diluent.

20. A hydroxypyrazolopyrimidine of formula (V) 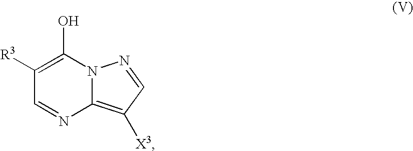
in which
R³ represents optionally substituted aryl, and
X³ represents halogen, cyano, nitro, alkyl, thiocarbamoyl, cycloalkyl, haloalkyl, alkoxycarbonyl, or alkylcarbonyl.

21. A process for preparing a hydroxypyrazolopyrimidine of formula (V) according to claim 20 comprising
(f) acrylic acid esters of the formula 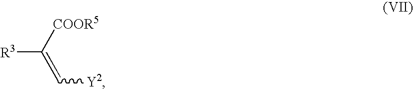
in which
R³ is as defined for formula (V) in claim 20,
R⁵ represents alkyl, and
Y² represents alkoxy or dialkylamino,
with an aminopyrazole of formula (VIII) 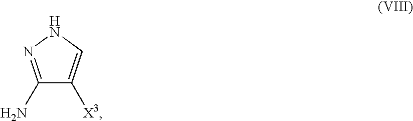
in which X³ is as defined for formula (V) in claim 20, optionally in the presence of a diluent and optionally in the presence of a base.

22. A dihydroxypyrazolopyrimidine of formula (VI) 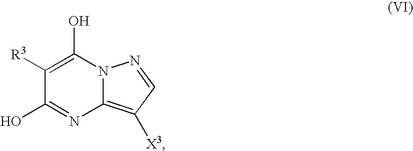
in which
R³ represents optionally substituted aryl, and
X³ represents halogen, cyano, nitro, alkyl, thiocarbamoyl, cycloalkyl, haloalkyl, alkoxycarbonyl, or alkylcarbonyl.

23. A process for preparing a dihydroxypyrazolopyrimidine of formula (VI) according to claim 22 comprising
(g) reacting a malonic ester of formula (IX) 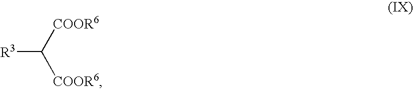
in which
R³ is as defined for formula (VI) in claim 22, and
R⁶ represents alkyl,
with an aminopyrazole of formula (VIII) 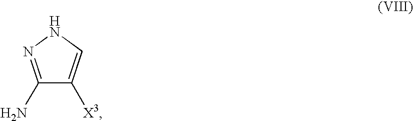
in which X³ is as defined for formula (VI) in claim 22, optionally in the presence of a diluent and optionally in the presence of a strong base.
